(19) United States
(12) Patent Application Publication
(10) Pub. No.: US 2025/0263738 A1
(43) Pub. Date: Aug. 21, 2025

(54) MODIFIED VIRUSES AND VIRAL PARTICLES, METHODS OF MAKING, AND USES THEREOF

(71) Applicant: The Regents of the University of California, Oakland, CA (US)

(72) Inventors: Prashant Mali, La Jolla, CA (US); Dhruva Katrekar, La Jolla, CA (US); Lauren Hodge, La Jolla, CA (US); Kyle M. Ford, La Jolla, CA (US); Andrew Portell, La Jolla, CA (US)

(21) Appl. No.: 18/285,000

(22) PCT Filed: Apr. 1, 2022
(86) PCT No.: PCT/US2022/023177

§ 371 Date: Sep. 29, 2023

Related U.S. Application Data
(60) Provisional application No. 63/170,100, filed on Apr. 2, 2021.

(51) Int. Cl. C12N 15/86; A61K 48/00; C40B 40/08

(52) U.S. Cl. CPC C12N 15/86; A61K 48/0041; C40B 40/08; C12N 2740/15043; C12N 2750/14122; C12N 2750/14143; C12N 2750/14145

(57) ABSTRACT

The disclosure provides methods for coating viruses and viral particles with membrane fragments to circumvent immune responses, the coated viruses and viral particles resulting therefrom, and the use of the coated viruses and viral particles in various applications, including gene therapy and genome engineering applications. The disclosure further provides methods for making ligand-modified viruses and viral particles, the ligand-modified modified viruses and viral particles resulting therefrom, and the use of the ligand-modified modified viruses and viral particles in various applications, including gene therapy and genome engineering applications.

### STATEMENT OF GOVERNMENT SUPPORT

This invention was made with Government support under Grant Nos. CA222826, GM123313, and HG009285, awarded by the National Institutes of Health. The Government has certain rights in the invention.

### CROSS REFERENCE TO RELATED APPLICATIONS

This application is a U.S. National Phase Application filed under 35 U.S.C. § 371 and claim priority to International Application No. PCT/US2022/023177, filed Apr. 1, 2022, which application claims priority under 35 U.S.C. § 119 from Provisional Application Ser. No. 63/170,100 filed Apr. 2, 2021, the disclosures of which are incorporated herein by reference.

### TECHNICAL FIELD

The disclosure provides methods for coating viruses and viral particles with membrane fragments to circumvent immune responses, the coated viruses and viral particles resulting therefrom, and the use of the coated viruses and viral particles in various applications, including gene therapy and genome engineering applications. The disclosure further provides methods for making ligand-modified viruses and viral particles, the ligand-modified modified viruses and viral particles resulting therefrom, and the use of the ligand-modified modified viruses and viral particles in various applications, including gene therapy and genome engineering applications.

### SEQUENCE LISTING

The instant application contains a Sequence Listing which has been submitted electronically in ASCII format and is hereby incorporated by reference in its entirety. Said ASCII copy, generated on Apr. 1, 2022, is named Sequence_ST25.txt and is 3,604,142 bytes in size.

### BACKGROUND

Viral gene therapy is a method to directly target mutations at a molecular level. Viruses are effective at delivering genes into the nucleus of the cell, but are easily recognized by the immune system, which can lead to increased side effects and rapid clearance. Adeno-associated virus (AAV) has been used increasingly as a promising vector for viral gene therapy. AAV is a small, non-enveloped virus that can transduce both dividing and quiescent cells, making it useful for many applications in viral gene therapy. The host's immune response to AAV is not a systemic response, and is limited to neutralizing antibodies, which leads to clearance, but no side effects. AAV is also nonpathogenic and therefore generally regarded as safe. Therefore, AAV has great potential in viral gene therapy if shielded from the immune system during transport.

There are currently multiple strategies being developed in an attempt to improve gene delivery by AAVs. A common method is shielding the AAV in a polymer such as polyethylene glycol (PEG). The difficulty with this strategy is that above an important ratio of polymer added to the virus, the transduction efficiency is significantly affected. Therefore, there is an upper limit to the amount of polymer that can be used to coat the viral vector; however, this upper limit is not sufficient to fully protect from the immune response. For example, one paper found that the upper limit of PEG that did not interfere with viral transduction was only protective against antibodies up to 6% of their normal concentration in human serum. Therefore, this method is not feasible in human trials, where the levels of antibodies in serum would completely neutralize the shielding effect. Also there have been recent studies indicating some patients receiving PEG coated nanoparticles develop antibodies against the polymer. Extracellular vesicles or exosomes naturally produced by cells have also been used to encapsulate AAVs, similarly synthetic lipid nanoparticles or liposomes have been used as a shield. These methods are able to protect against antibodies at higher concentrations than the polymer coating method however the transduction efficiencies often decreased. In general, it appears the larger size of these vesicle or exosomes bound AAVs hinders their ability to disseminate throughout the body and infect a wide range of cell types. For each of these methods, the addition of certain proteins or other molecules have been investigated as a way to improve efficiency, and while cell targeting and transduction efficiencies can be improved, the neutralizing effects of antibodies still present a challenge.

### SUMMARY

The disclosure provides a viral vector having a capsid protein comprising a heterologous targeting peptide in a range of 10-30 amino acids in length. In one embodiment, the heterologous targeting peptide is about 15-25 amino acids in length. In another or further embodiment, the heterologous targeting peptide is about 20 amino acids in length. In another or further embodiment, the viral vector is an adeno-associated virus (AAV). In another or further embodiment, the viral vector is a lentiviral vector. In another or further embodiment, the capsid protein is a VP1 capsid protein. In another or further embodiment, the capsid protein is a VP2 capsid protein. In another or further embodiment, the capsid protein is a VP3 capsid protein. In another or further embodiment, the heterologous targeting peptide is inserted into an AAV capsid protein at loop 1 and/or loop 2. In another or further embodiment, the viral vector is an AAV5. In another or further embodiment, the viral vector is an AAV9). In another or further embodiment, the heterologous targeting peptide is flanked by a linker peptide at the N-terminal and C-terminal ends of the heterologous targeting peptide. In another or further embodiment, the heterologous targeting peptide is flanked by a linker peptide at the N-terminal and C-terminal ends of the heterologous targeting peptide. In another or further embodiment, the heterologous targeting peptide targets the viral vector to hepatocytes or liver tissue. In another or further embodiment, the heterologous targeting peptide targets the viral vector to neuronal cells or brain tissue. In another or further embodiment, the heterologous targeting peptide targets the viral vector to pancreatic cells or pancreas tissue. In another or further embodiment, the heterologous targeting peptide targets the viral vector to cardiac cells or heart tissue. In another or further embodiment, the heterologous targeting peptide targets the viral vector to lung tissue. In another or further embodiment, the heterologous targeting peptide targets the viral vector to intestinal tissue. In another or further embodiment, the heterologous targeting peptide targets the viral vector to spleen tissue. In another or further embodiment, the heterologous targeting peptide targets the viral vector to renal cells or kidney tissue. In another or further embodiment, the heterologous targeting peptide targets the viral vector to muscle cells or tissue.

The disclosure also provides an adeno-associated virus (AAV) capsid protein comprising a heterologous targeting peptide cloned into loop 1 and/or loop 2 of the capsid protein, wherein the heterologous targeting peptide is about 10-30 amino acids in length. In one embodiment, the capsid protein is a VP1 capsid protein. In another embodiment, the capsid protein is a VP2 capsid protein. In still another embodiment, the capsid protein is a VP3 capsid protein. In another or further embodiment, the heterologous targeting peptide is about 15-25 amino acids in length. In another or further embodiment, the heterologous targeting peptide is about 20 amino acids in length. In another or further embodiment, the heterologous targeting peptide is flanked by a linker peptide at the N-terminal and C-terminal ends of the heterologous targeting peptide. In another or further embodiment, the heterologous targeting peptide targets hepatocytes or liver tissue. In another or further embodiment, the heterologous targeting peptide targets neuronal cells or brain tissue. In another or further embodiment, the heterologous targeting peptide targets pancreatic cells or pancreas tissue. In another or further embodiment, the heterologous targeting peptide targets cardiac cells or heart tissue. In another or further embodiment, the heterologous targeting peptide targets lung tissue. In another or further embodiment, the heterologous targeting peptide targets intestinal tissue. In another or further embodiment, the heterologous targeting peptide targets spleen tissue. In another or further embodiment, the heterologous targeting peptide targets renal cells or kidney tissue. In another or further embodiment, the heterologous targeting peptide targets muscle cells or tissue.

The disclosure also provides recombinant AAV (rAAV) comprising a capsid protein of the disclosure comprising a targeting peptide.

The disclosure also provides a recombinant AAV (rAAV) comprising a capsid protein having a targeting peptide in loop 1 and/or loop 2 wherein the targeting peptide is independently selected from SEQ ID Nos:5865 to 11445. In another or further embodiment, the recombinant AAV further comprises a heterologous polynucleotide for gene delivery In another or further embodiment, the heterologous polynucleotide is a therapeutic gene.

The disclosure also provides a composition comprising the recombinant rAAV of the disclosure. In one embodiment, the composition further comprises a pharmaceutically acceptable carrier.

The disclosure also provides a method for delivering a transgene to a subject comprising: administering a recombinant AAV (rAAV) to a subject, wherein the rAAV comprises: (i) a capsid protein of the disclosure comprising a targeting peptide, and (ii) at least one transgene, and wherein the rAAV infects cells of a target tissue of the subject. In another or further embodiment, the at least one transgene encodes a protein. In another or further embodiment, the protein is an immunoglobulin heavy chain or light chain or fragment thereof. In another or further embodiment, the at least one transgene encodes a small interfering nucleic acid. In another or further embodiment, the small interfering nucleic acid is a miRNA. In another or further embodiment, the small interfering nucleic acid is a miRNA sponge or TuD RNA that inhibits the activity of at least one miRNA in the subject or animal. In another or further embodiment, the miRNA is expressed in a cell of the target tissue. In another or further embodiment, the target tissue is skeletal muscle, heart, liver, pancreas, brain or lung. In another or further embodiment, the transgene expresses a transcript that comprises at least one binding site for a miRNA, wherein the miRNA inhibits activity of the transgene, in a tissue other than the target tissue, by hybridizing to the binding site. In another or further embodiment, the at least one transgene encodes a gene product that mediates genome editing. In another or further embodiment, the transgene comprises a tissue specific promoter or inducible promoter. In another or further embodiment, the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. In another or further embodiment, the rAAV is administered intravenously, intravascularly, transdermally, intraocularly, intrathecally, orally, intramuscularly, subcutaneously, intranasally, or by inhalation. In another or further embodiment, the subject is selected from a mouse, a rat, a rabbit, a dog, a cat, a sheep, a pig, and a non-human primate. In another or further embodiment, the subject is a human.

The disclosure provides an isolated nucleic acid encoding an AAV capsid protein containing an amino acid sequence selected from the group consisting of SEQ ID No:5865 to 11444 and 11445. The disclosure also provides a composition comprising the isolated AAV capsid protein.

The disclosure also provides a kit for producing a rAAV, the kit comprising: a container housing an isolated nucleic acid encoding a capsid protein comprising a targeting peptide of the disclosure. In one embodiment, the kit further comprises instructions for producing the rAAV. In another or further embodiment, the kit further comprises at least one container housing a recombinant AAV vector, wherein the recombinant AAV vector comprises a transgene.

The disclosure also provides a method for coating a virus or viral particle with membrane fragments comprising: lysing donor cells in a hypotonic solution, which optionally may be combined with Dounce homogenization or sonication, in order to fractionate the cell membrane; removing cells and cell debris by one or more rounds of centrifugation, leaving a membrane enriched fraction; extruding the membrane enriched faction through polycarbonate membrane(s) to generate purified membrane fragments; and coating virus or viral particles by coextruding the virus or viral particles with the purified membrane fragments through polycarbonate membrane(s). In another or further embodiment, the viruses or viral particles are non-enveloped viruses or viral particles. In another or further embodiment, the viruses or viral particles are enveloped viruses or viral particles which have had their viral envelope removed. In another or further embodiment, the viruses or viral particles are selected from retroviruses, adenovirus, adeno-associated virus, hybrid adenoviruses, alphavirus, herpes simplex virus, poxvirus, Epstein-Barr virus and lentivirus. In another or further embodiment, the viruses or viral particles are adeno-associated viruses (AAV). In another or further embodiment, the viruses or viral particles have been modified by directed evolution to have increased neutralizing antibody-evasion properties, as well as enhanced gene delivery, gene targeting, and/or enhanced capacity to infect. In another or further embodiment, the viruses or viral particles have been modified by one or more amino acid substitutions in one or more regions of a viral capsid protein so as to reduce the affinity of the viral capsid protein for the major histocompatibility complex. In another or further embodiment, the donor cells are mammalian cells. In another or further embodiment, the donor cells are human cells. In another or further embodiment, the donor cells are human stem cells, human progenitor cells, human primary cells, human somatic cells, human germline cells, or human tumor cells. In another or further embodiment, the membranes of the donor cells have been modified to express or present a targeting ligand. In another or further embodiment, the targeting ligand is used to improve entry of the coated viruses or viral particles into target cells, inhibit components of the immune response to the coated viruses or viral particles, or to target the coated viruses or viral particles to certain cell types or organs In another or further embodiment, the targeting ligand is a peptide, antibody or antibody fragment. In another or further embodiment, the targeting ligand comprises a peptide of any one of SEQ ID Nos:5865 to 11445.

The disclosure also provides coated viruses or viral particles made by the method described above. In another or further embodiment, the coated viruses or viral particles have been modified to comprise a targeting ligand. In another or further embodiment, the coated viruses or viral particles are used to deliver transgene(s) into target cells. In another or further embodiment, the coated viruses or viral particles are used to genome engineer target cells.

The disclosure also provides a pharmaceutical composition comprising the coated viruses or viral particles and a pharmaceutically acceptable carrier, diluent, binder and/or filler.

The disclosure also provides a method of treating a subject suffering from a disease or disorder in need of treatment thereof, comprising administering the coated viruses or viral particles or the pharmaceutical composition of the disclosure.

The disclosure also provides an engineered viral particle comprising an artificially prepared lipid envelope.

The disclosure provides a method of preparing an engineered retroviral particle, the method comprising treating a retroviral particle with a detergent to remove a lipid envelop to obtain naked retroviral particles, isolating the naked retroviral particles and co-extruding a lipid envelop with the naked retroviral particles to obtain an engineered retroviral particle.

### DESCRIPTION OF DRAWINGS

FIG. 1A-B provides embodiment of methodologies that can be used to (A) prepare cell membrane fragments for (B) coating adeno-associated viruses (AAV).

FIG. 2 illustrates how the coated AAVs of the disclosure can provide for higher transgene expression in vivo by minimizing immune detection and clearance.

FIG. 3 presents the standard recognized model of the relationship between ‘uncoated’ AAV capsid dose and outcome of gene transfer following systemic vector delivery. Low ‘uncoated’ capsid doses are more likely to be neutralized by anti-AAV antibodies, even low-titer NAb. This results in lack of efficacy. Higher ‘uncoated’ capsid doses overcome this limitation, leading to therapeutic efficacy. Capsid-specific T-cell activation is detected as the total uncoated capsid dose administered increases. This does not affect efficacy until an important threshold is reached, above which immune-mediated clearance of transduced target cells results in loss of efficacy. The ‘coated’ AAVs of the disclosure, unlike the ‘uncoated’ AAVs described above and presented in FIG. 3, can achieve efficacy at much lower doses, as the ‘coated’ AAVs made by the methods presented herein are far less likely to be neutralized by anti-AAV antibodies.

FIG. 4 illustrates how the coated AAVs of the disclosure can be engineered to have tissue specificity using targeting ligands, thereby providing for programmable tropism.

FIG. 5 shows an overall workflow for rationally engineering AAV variants and screening them in vivo. The pie chart depicts the distribution of categories from which protein sources of peptides were selected. These proteins were tiled into 20-mer peptides and synthesized on an oligonucleotide pool. DNA coding for the peptides were then inserted into distinct locations on the AAV capsid enabling production of AAV5 and AAV9 variants for a total of ˜1.1 million capsid variants. AAVs were then injected retro-orbitally into replicate mice. Two weeks later, organs, including the liver, spleen, brain, large intestine, lung, kidney, heart, skeletal muscle, and pancreas were harvested. DNA was isolated from these tissues, the peptide insertion region was selectively PCR-amplified and prepared for sequencing, and then paired-end 100 deep sequencing was performed using the Illumina NovaSeq platform to analyze transducing variants.

FIG. 6A-B shows engineering peptide-displaying AAV variants. (a) Icosahedral structural rendering of the AAV5 capsid. Surface residues are colored according to their distance from the capsid center with specific amino acid residues highlighted to illustrate the location of the Loop1 (red) and Loop2 (salmon) inserts. (b) Cloning strategy shown for inserting the peptide library into the wild-type AAV backbone with flanking G-S residues, AAV5-Loop1 shown as an example. The AAV backbone was modified at the desired location to insert two DNA sequences encoding Glycine and Serine, along with two PaqCI type IIS restriction sites flanking an approximately 60-base pair filler region to be cut out. The peptide insert library is flanked by two PaqCI recognition sites for ligation upon restriction digest.

FIG. 7A-D shows plasmid and capsid level analysis. Polar plots illustrating the proportion of peptide library recovered after cloning the library into the cap gene and then packing the plasmid pool into functional AAV capsids for (a) AAV5-Loop1, (b) AAV5-Loop2, (c) AAV9-Loop1, and (d) AAV9-Loop2. Numbers on the perimeter represent total peptides quantified.

FIG. 8A-E shows a method of Identifying top transducing AAV variants. (a) On the left, a heatmap illustrating the AAV variants across all capsids and loops which have a log2fc>1 in both replicates, and FDR adjusted p<0.05 (one sample T test comparing capsid counts to organ counts). Shown to the right is a heatmap showing the levenshtein distance between the peptides in the left heatmap. The data has been filtered to remove peptides with no detected homology within the dataset. (c-e) Heatmaps showing final hits which have a Z-score (Z-normalized log2FC) greater than 2.5 in any organ, a log2(capsid count)>3, and at least one homologous peptide detected in the same organ. Heatmaps are separated by AAV serotype/insertion site.

FIG. 9A-B shows identifying pan-organ and organ specific AAV variants. (a) Pan-organ transducing AAV variants. Pan-organ specific AAVs were identified by taking the average log2FC across all organs for each capsid/loop. Shown in the heatmap are all variants which have an average (across all organs) log2FC greater than 1. (b) Organ specific AAV variants. Organ specific AAVs were identified via an ANOVA test, comparing the log2FC values in one organ versus all the others. AAV variants were then ranked to identify the variants with the lowest p-values. The log2FC values for the AAV variants with the 5 lowest p values for each organ are plotted in the heatmap.

FIG. 10A-C shows an overview of lentiviral display strategy. (a) Plasmid map showing key genetic material packaged into lentiviral particles. Each lentiviral particle contains the RNA coding for a displayed peptide, as well as a puromycin resistance gene under the control of the same promoter. (b) Cartoon diagram of engineered lentivirus, showing displayed peptides and mutant VSVG protein. (c) Cartoon diagram showing cell lines of interest screened via lentiviral display strategy. Cell lines cover a variety of lineages and tissue types to enable development of tissue specific lentiviral particles.

FIG. 11 provides a table of AAV targeting hits in the indicated tissues.

FIG. 12 provides a table of Lentivirus targeting hits in the indicated cells.

FIG. 13 presents a wild type DNA and peptide sequences for AAV5. Further indicated is the Loop 1 and Loop 2 insertion sites in the wildtype sequences for AAV5, as highlighted in lighter gray and medium gray, respectively.

FIG. 14 presents a wild type DNA and peptide sequences for AAV9. Further indicated is the Loop 1 and Loop 2 insertion sites in the wildtype sequences for AAV9, as highlighted in lighter gray and medium gray, respectively.

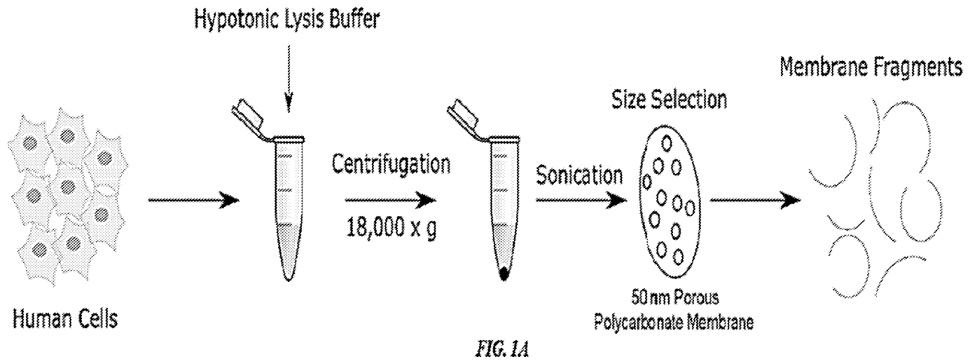

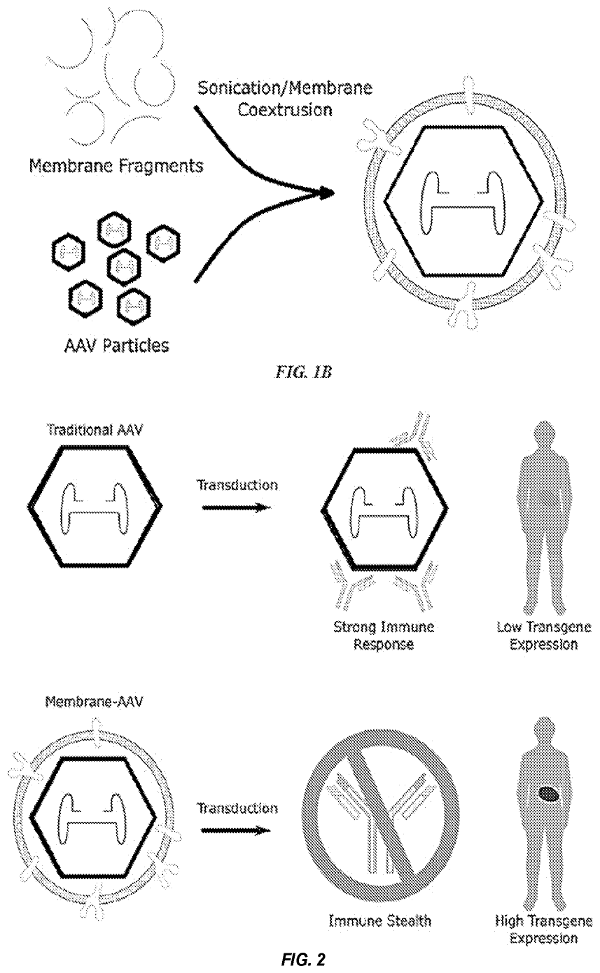

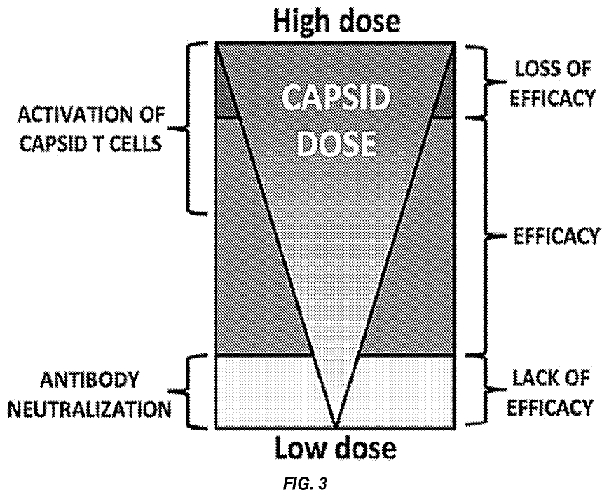

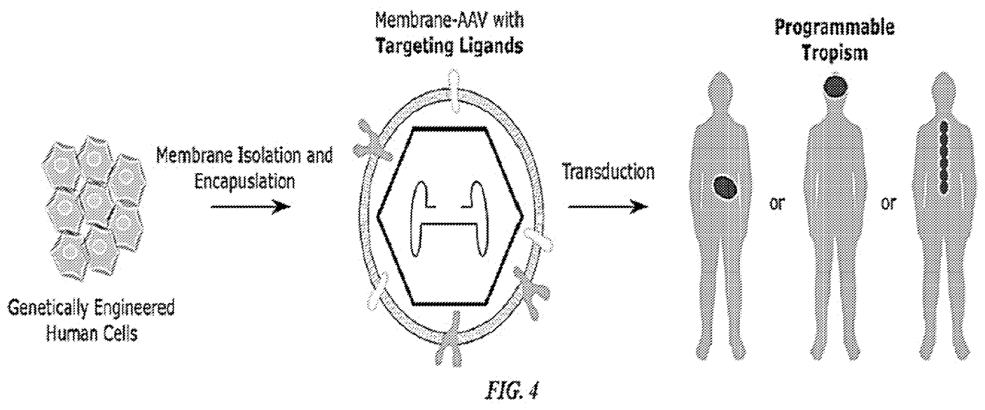

### DETAILED DESCRIPTION

As used herein and in the appended claims, the singular forms “a,” “an,” and “the” include plural referents unless the context clearly dictates otherwise. Thus, for example, reference to “a virus” includes a plurality of such viruses and reference to “the viral particle” includes reference to one or more viral particles and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%.

The term “purified” when used in reference to viruses or viral particles disclosed herein refers to the fact that the virus is removed from the majority of other cellular components from which it was generated or in which it is typically present in nature, or from the coating agents disclosed herein. The coated viruses or viral particles disclosed herein are typically prepared to the state where they are purified or semi-purified.

An “effective amount” as the term is used herein, is used to refer to an amount that is sufficient to produce at least a reproducibly detectable amount of the desired results. An effective amount will vary with the specific conditions and circumstances. Such an amount can be determined by the skilled practitioner for a given situation.

The term “therapeutically effective amount” refers to an amount that is sufficient to affect a therapeutically significant reduction in one or more symptoms of the condition when administered to a typical subject who has the condition. A therapeutically significant reduction in a symptom is, e.g. about 10%, about 20%, about 30%>, about 40%>, about 50%, about 60%, about 70%, about 80%, about 90%, about 100%, or more as compared to a control or non-treated subject.

The term “treat” or “treatment” refers to therapeutic treatment wherein the object is to eliminate or lessen symptoms. Beneficial or desired clinical results include, but are not limited to, elimination of symptoms, alleviation of symptoms, diminishment of extent of condition, stabilized (i.e., not worsening) state of condition, delay or slowing of progression of the condition.

The terms “patient”, “subject” and “individual” are used interchangeably herein, and refer to an animal, particularly a human, to whom treatment including prophylactic treatment is provided. This includes human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. A subject can be male or female. A subject can be a fully developed subject (e.g., an adult) or a subject undergoing the developmental process (e.g., a child, infant or fetus).

Gene therapy is the process of introducing foreign genomic materials into host cells to elicit a therapeutic benefit. Although initially the main focus of gene therapy was on special genetic disorders, now diverse diseases with different patterns of inheritance and acquired diseases are targets of gene therapy. Basically, gene therapy is an intracellular delivery of genomic materials (transgene) into specific cells to generate a therapeutic effect by correcting an existing abnormality or providing the cells with a new function. Different types of gene delivery systems may be applied in gene therapy to restore a specific gene function or turning off a special gene(s). The ultimate goal of gene therapy is single administration of an appropriate material to replace a defective or missing gene. One of the successful gene therapy systems available today are viral vectors, such as retrovirus, adenovirus (types 2 and 5), adeno-associated virus, herpes virus, pox virus, human foamy virus (HFV), and lentivirus. All viral vector genomes have been modified by deleting some areas of their genomes so that their replication becomes deranged and it makes them safer, but the system has some problems, such as their marked immunogenicity that causes induction of inflammatory system leading to degeneration of transduced tissue; and toxin production, including mortality, the insertional mutagenesis; and their limitation in transgenic capacity size. During the past few years some viral vectors with specific receptors have been designed that could transfer the transgenes to some other specific cells, which are not their natural target cells (retargeting).

Adenoviral vectors have been isolated from a large number of different species, and more than 100 different serotypes have been reported. Most adults have been exposed to the adenovirus serotypes most commonly used in gene therapy (types 2 and 5). Adenoviruses type 2 and 5 can be utilized for transferring both dividing and nondividing cells and have low host specificity so can be used for gene delivery into large range of tissues.

Adeno-associated vectors (AAV) are like adenoviral vectors in their features but because of having some deficiency in their replication and pathogenicity, are safer than adenoviral vectors. In human, AAVs are not associated with any disease. Another special character of AAV is their ability to integrate into a specific site on chromosome 19 with no noticeable effects cause long-term expression in vivo. The major disadvantages of these vectors are complicated process of vector production and the limited transgene capacity of the particles (up to 4.8 kb). AAVs have been used in the treatment of some diseases, such as CF, hemophilia B, Leber congenital amaurosis, and AAT (Alpha-1 antitrypsin) deficiency.

Current AAV gene delivery system does not allow for repeated treatments, due to the immune response generated in the patient if a second dose is attempted. Currently there are methods of shielding AAVs to avoid this scenario but experimental data shows that there is an improvement only for avoiding small amounts of neutralizing antibodies. As stated above, methods like coating with polymers face a challenge of the proper ratio of AAV to polymer. If the polymer concentration is too high then the ability of the AAV to enter the cell is compromised, but if the polymer concentration is low enough to preserve the transduction efficiency, then there is little protection against antibodies. Vesicle and exosome encapsulation can also be a way to shield the AAV from the immune system but the increase in size generally reduces the ability of the AAV to infect a broad range of cell types, which traditionally was one of the advantages to using AAVs. Therefore, these methods are not effective in normal physiological conditions. One of the main advantages of AAVs is their small size (25 nm) which allows them to transduce most tissues of the body. The strategies employing native vesicles may help get around the response of the immune system but the increased size negatively impacts transduction efficiencies.

The disclosure provides methods that can be used to coat AAV and other viruses by using fragments of purified cell membranes to coat the viruses. As such, the methods of the disclosure are especially suited to coating naturally occurring non-enveloped viruses, like adenoviruses or AAVs. For example, one can coat a non-enveloped AAV using the methods disclosed herein, thereby providing for a coated AAV of a specific size that has the surface properties of a native cell with the advantages of small size. Additionally, the methods of the disclosures can be used with enveloped viruses, like retroviruses. In such a case, the envelope of the virus may be first removed using standard methods, like detergent treatment, and then be coated with membrane fragment using the methods disclosed herein. In doing so, new, non-standard applications for the ‘coated’ virus can be possibly realized, such as an increased host range, and programmable tropism. Accordingly, the methods of the disclosure allow for, e.g., the production of coated viruses or viral particles that are recognized as self by the immune system, thereby preventing an immunogenic response, while also retaining the ability to transduce many different cell types.

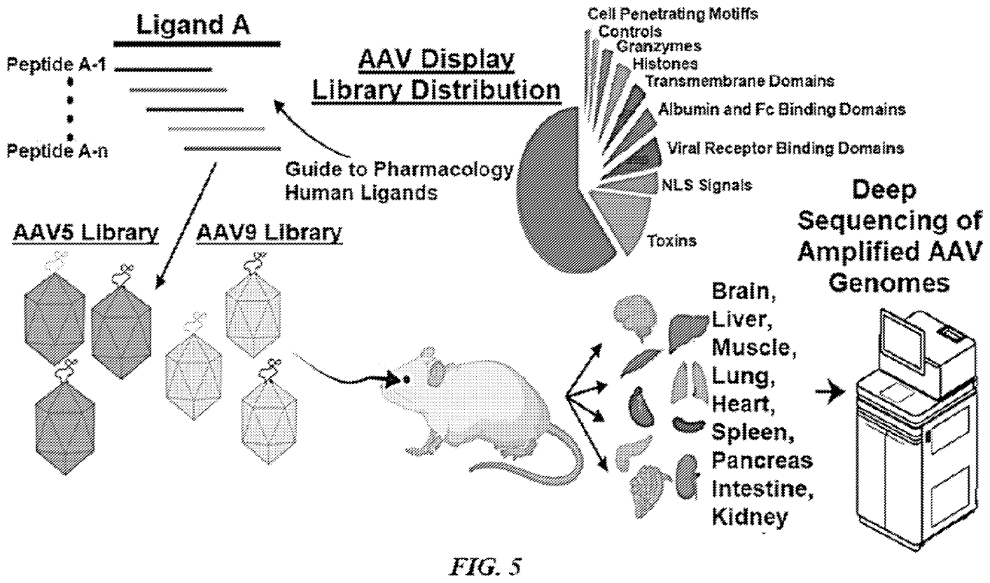

The disclosure further provides methods for rationally engineering novel viral variants (e.g., AAV variants) and identifying transducing capsids which exhibit strong activity and organ specificity in vivo (see FIG. 5). In a particular embodiment, the disclosure provides a viral vector having a capsid protein comprising a heterologous targeting peptide of 10, 11, 12. 13, 14, 15, 16, 17, 18, 19, 20, 21, 22, 23, 24, 25, 26, 27, 28, 29, 30 amino acids in length, or a range that includes or is between any two of the foregoing amino acid lengths (e.g., 10-30 amino acids in length, 15-25 amino acids in length, etc.). In a particular embodiment, the heterologous targeting peptide is about 20 amino acids in length. In regards to the transducing capsid, the capsid can be of any type of capsid proteins. including but not limited to, VP1, VP2, VP3, N protein, and HHV capsid portal protein. In a particular embodiment, the capsid protein is a VP1 capsid protein. In another embodiment, the capsid protein is a VP2 capsid protein. In yet another embodiment, the capsid protein is a VP3 capsid protein. Examples of viruses and viral particles, or vectors encoding thereof, which can be used in the methods of the disclosure include, but are not limited to, retroviruses, adenovirus, adeno-associated virus, hybrid adenoviruses, alphavirus, herpes simplex virus, poxvirus, Epstein-Barr virus and lentivirus.

Adenoviruses are able to deliver large DNA particles (up to 38 kb), but in contrast to retroviruses, as they would not integrate into the host genome, their gene expression is too short term. Natural and acute immunologic responses against adenoviruses have made their clinical application limited to a few tissues, such as liver, lung (especially for CF (Cystic Fibrosis) treatment), or localized cancer gene therapy. Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988): Rosenfeld et al., Science 252:431-434 (1991); and Rosenfeld et al., Cell 68: 143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986)).

Retroviruses are one of the most frequently employed forms of gene delivery in somatic and germline gene therapies. Retroviruses in contrast to adenoviral and lentiviral viruses, can transfect dividing cells because they can pass through the nuclear pores of mitotic cells; this character of retroviruses make them proper candidates for in situ treatment. In addition, all of the viral genes have been removed, creating approximately 8 kb of space for transgenic incorporation. Retroviruses are useful for ex vivo delivery of somatic cells because of their ability to linearly integrate into host cell genome; for example, they have been used for human gene therapy of X-SCID successfully but incidence of leukemia in some patients occurred because of integration of retroviruses to the LMO2 gene and inappropriate activation of it. Retroviruses also have been applied for familial hyperlipidemia gene therapy and tumor vaccination. However, the main limitations of retroviruses are their low efficiency in vivo, immunogenic problems, the inability to transduce the nondividing cells and the risk of insertion, which could possibly cause oncogene activation or tumor-suppressor gene inactivation. A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al, eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include Ψ&\acute{ι}ρ, Ψ&ε, Ψ2 and ΨAπι. Retroviruses have been used to introduce a variety of genes into many different cell types, including epithelial cells, in vitro and/or in vivo (see for example Eglitis, et al. (1985) Science 230: 1395-1398: Danos and Mulligan (1988) Proc. Natl. Acad. Sci. USA 85:6460-6464; Wilson et al. (1988) Proc. Natl. Acad. Sci. USA 85:3014-3018: Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381: Chowdhury et al. (1991) Science 254: 1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89: 10892-10895; Hwu et al. (1993) J. Immunol. 150:4104-4115: U.S. Pat. Nos. 4,868,116; 4,980,286; PCT Application WO 89/07136; PCT Application WO 89/02468; PCT Application WO 89/05345; and PCT Application WO 92/07573).

Hybrid adenoviruses are made of the high transduction efficiency of a gene-deleted adenoviral vector and the long-term genome-integrating potential of adeno-associated and retroviruses viruses. Such hybrid systems show stable transduction and limited integration sites. Among integrating vectors, those derived from retroviruses are most common. One of the family of Retroviridae are called spuma retroviruses or foamy viruses (FVs). FVs are a group of apparently nonpathogenic nonhuman retroviruses, which have been developed only recently. The potential advantages of FV vectors include a broad range of hosts, the largest packaging capacity of any retrovirus, and the ability to persist in quiescent cells. Because of these features, FVs have the unique potential to safely and efficiently deliver several genes into a number of different types of cells.

Alphaviruses can also be used. Alphaviruses are enveloped single stranded RNA viruses that have a broad host range, and when used in viral gene therapy protocols alphaviruses can provide high-level transient gene expression. Exemplary alphaviruses include the Semliki Forest virus (SFV), Sindbis virus (SIN) and Venezuelan Equine Encephalitis (VEE) virus, all of which have been genetically engineered to provide efficient replication-deficient and -competent expression vectors. Alphaviruses exhibit significant neurotropism, and so are useful for CNS-related diseases. See, e.g., Lundstrom, Viruses. 2009 June; 1(1); 13-25; Lundstrom, Viruses. 2014 June; 6(6): 2392-2415; Lundstrom, Curr Gene Ther. 2001 May; 1(1): 19-29; Rayner et al., Rev Med Virol. 2002 September-October; 12(5):279-96.

Herpes simplex virus (HSV) is one of the recent viruses candidate in gene delivery. HSV systems include the development of the so-called disabled infectious single copy (DISC) viruses, which comprise a glycoprotein H defective mutant HSV genome. When the defective HSV propagated in complementing cells' viral particles are generated, they can infect in subsequent cells permanently replicating their own genome but not producing more infectious particles. Herpes vectors can deliver up to 150 kb transgenic DNA and because of its neuronotropic features, it has the greatest potential for gene delivery to nervous system, tumors, and cancer cells.

Epstein-Barr virus as a herpes virus can be used for the expression of large DNA fragments in target cells. Because Epstein Barr virus (EBV) establishes itself in the host nucleus in a latent state as extrachromosomal circular plasmid, this virus is suitable for long-term retention in the target cell. Because of the natural B-cell tropism of the virus, EBV-derived vectors, such as B-cell lymphoma, have been tested for immune therapy of cancer.

Poxvirus vectors are members of the Poxviridae family that are widely used for high-level cytoplasmatic expression of transgenes. The high stable insertion capacity (more than 25 KB) of this virus is the most advantageous feature of it for gene delivery. The insertion of the transgene sequences is somewhat different from the other vector systems and utilizes homologous recombination or in vitro ligation for construction of recombinant vaccinia virus vectors. Poxviruses have been used for cancer therapy in various studies, such as prostate cancer, colorectal cancer, breast cancer, and lung cancer. Recombinant vaccinia virus vectors were also used for expression of E6 and E7 genes of human papilloma virus types 16 and 18 in cervical cancer patients to induce tumor regression.

Lentiviruses are a subclass of retroviruses. They have recently been used as gene delivery vectors due to their ability to naturally integrate with nondividing cells, which is the unique feature of lentiviruses as compared with other retroviruses, which can infect only the dividing cells. Lentiviral vectors can deliver 8 kb of sequence. Because lentiviruses have strong tropism for neural stem cells, extensively used for ex vivo gene transfer in central nervous system with no significant immune responses and no unwanted side effects. Lentiviral vectors have the advantages of high-efficiency infection of dividing and nondividing cells, long-term stable expression of a transgene, low immunogenicity, and the ability to accommodate larger transgenes. There are numerous examples of effective long-term treatment of animal models of neurologic disorders, such as motor neuron diseases, Parkinson, Alzheimer, Huntington's disease, lysosomal storage diseases, and spinal injury.

Adeno-associated viruses, from the parvovirus family, are small viruses with a genome of single stranded DNA. AAV was discovered in 1960s as a contaminant in adenovirus (a cold causing virus) preparations. Its growth in cells is dependent on the presence of adenovirus and, therefore, it was named as adeno-associated virus. AAV can infect both dividing and non-dividing cells and may incorporate its genome into that of the host cell. These features make AAV a very attractive candidate for creating viral vectors for gene therapy. The AAV viruses can insert genetic material at a specific site on chromosome 19 with near 100% certainty. There are a few disadvantages to using AAV, including the small amount of DNA it can carry (low capacity) and the difficulty in producing it. This type of virus is being used for gene therapy, however, because it is non-pathogenic (most people carry this harmless virus). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. For example, AAV9 has been shown to efficiently cross the blood-brain barrier. Moreover, the AAV capsid can be genetically engineered to increase transduction efficiency and selectivity, e.g., biotinylated AAV vectors, directed molecular evolution, self-complementary AAV genomes and so on. Modified AAV have also been described, including AAV based on ancestral sequences; see, e.g., U.S. Pat. No. 7,906,111; WO/2005/033321; WO2008027084, WO2014124282; WO2015054653; and WO2007127264.

In certain embodiment, the disclosure provides for the generation of modified AAVs engineered to contain heterologous targeting peptides that target the AAVs to certain cells and/or tissues. Modified AAVs disclosed herein are useful because they can effectively deliver nucleic acids of interest to a particular cell and/or tissue, e.g., for purposes of manipulating levels of a particular gene product in the cell and/or tissue. For example, in some embodiments, the disclosure provides modified AAVs comprising a capsid protein having a heterologous targeting peptide that confers unique tissue targeting and cell transduction properties. In some embodiments, such heterologous targeting peptides are useful for targeting AAVs to tissues of the central nervous system (CNS) (e.g., the brain), liver, muscle, lung, heart, spleen pancreas, intestine and/or kidney. In a further embodiment, the AAV used in the compositions, methods, and kits disclosed herein has a serotype selected from AAV1, AAV2, AAV3, AAV4, AAV5, AAV6, AAV7, AAV8, AAV9, AAV9.47, AAVrh10, AAVPHP.B, AAVPHP.eB, and AAVPHP.S. In a certain embodiment, the AAV used in the compositions, methods, and kits herein is AAV5. In another embodiment, the AAV used in the compositions, methods, and kits disclosed herein is AAV9.

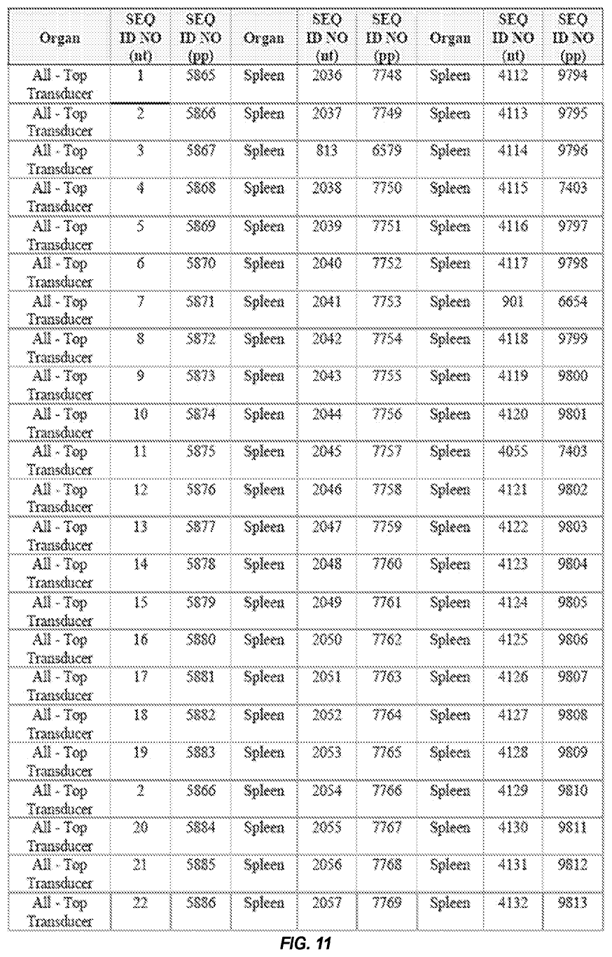

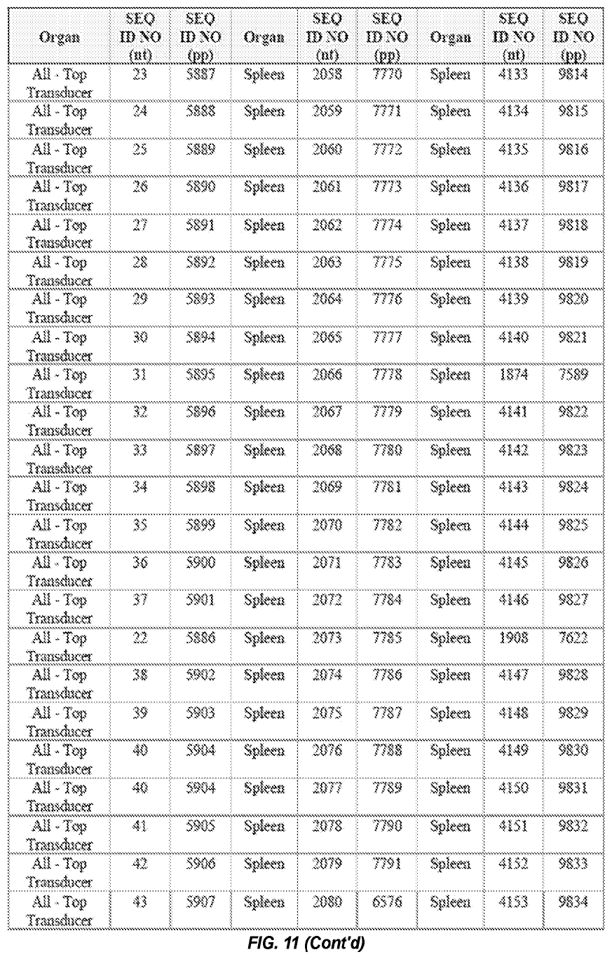

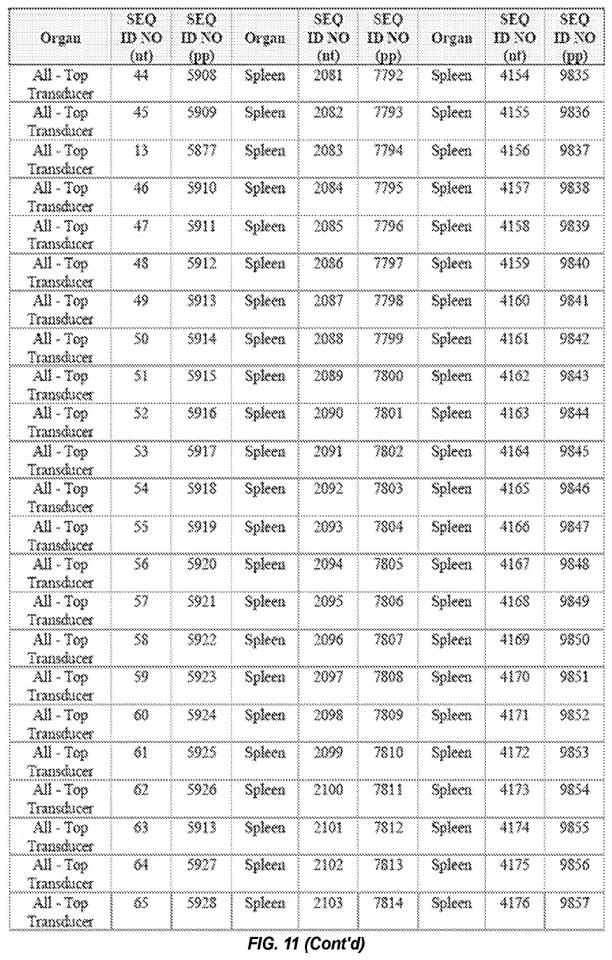

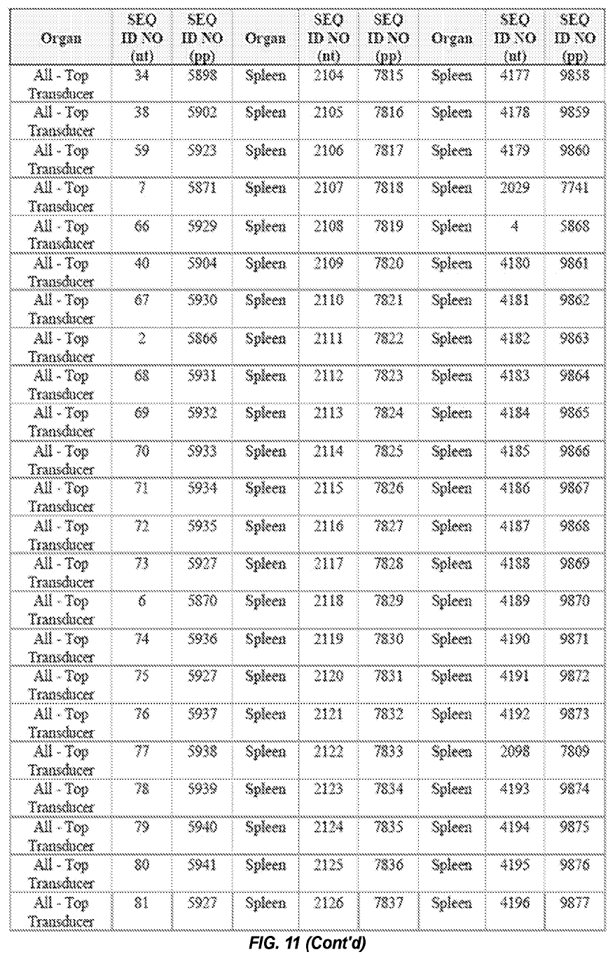

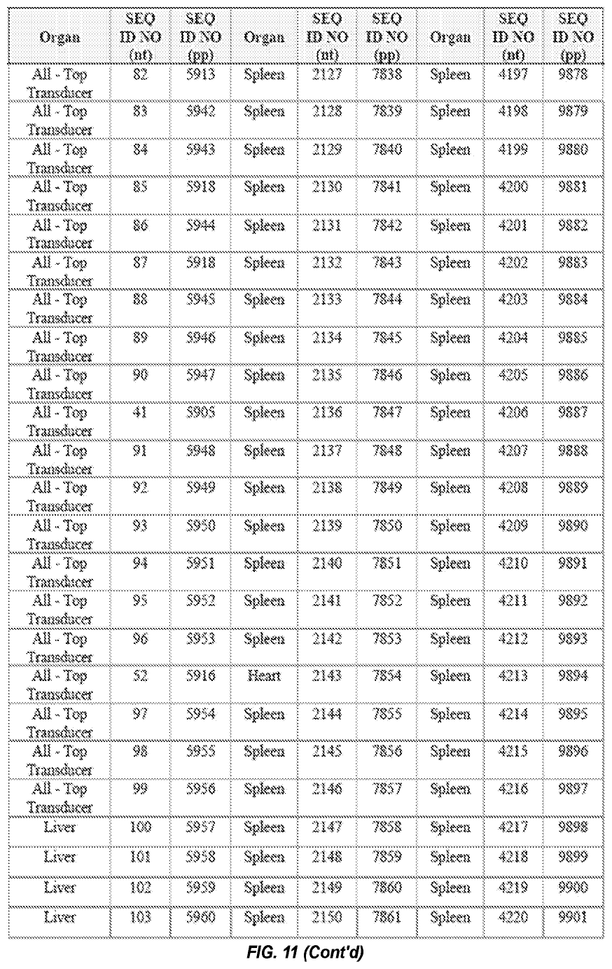

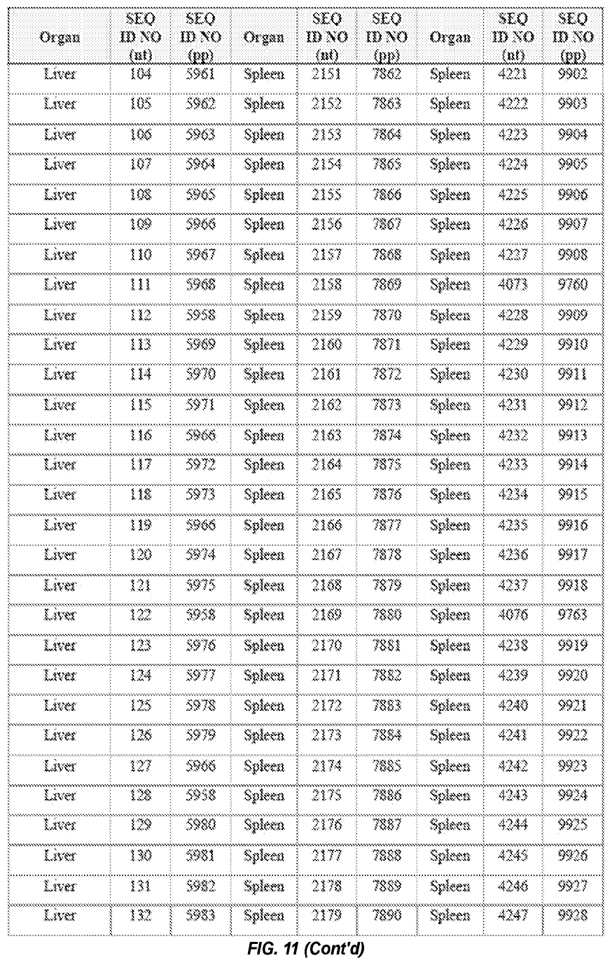

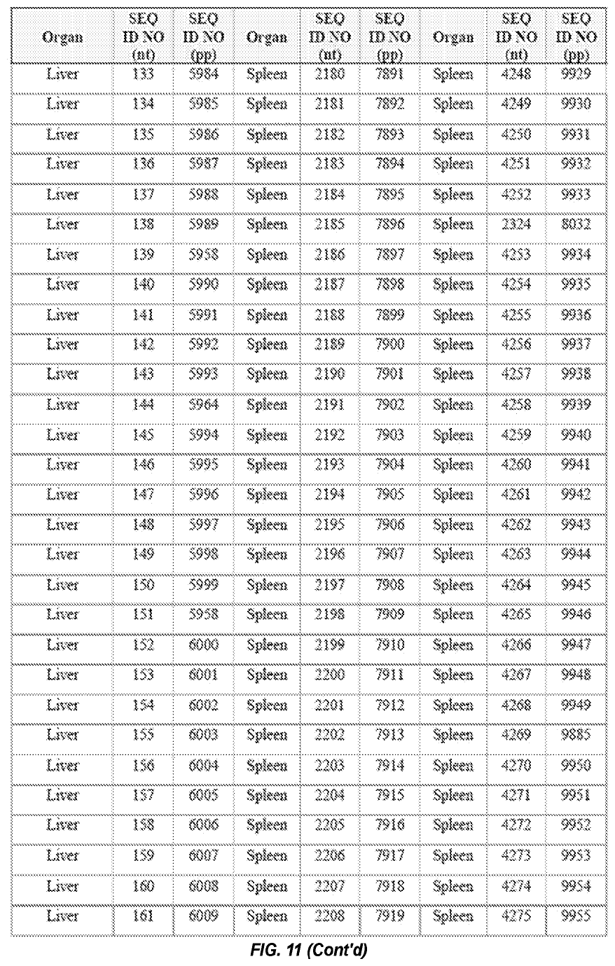

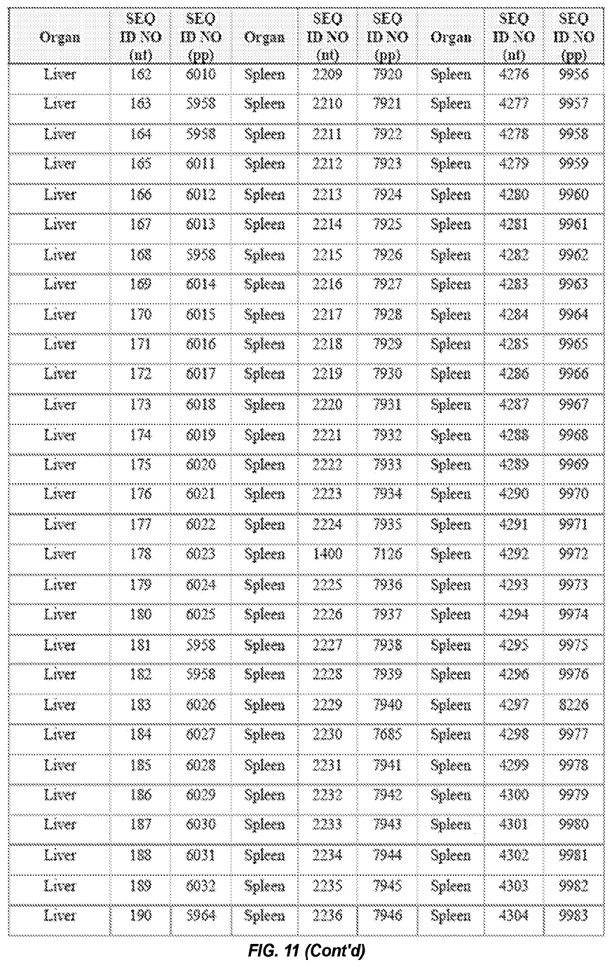

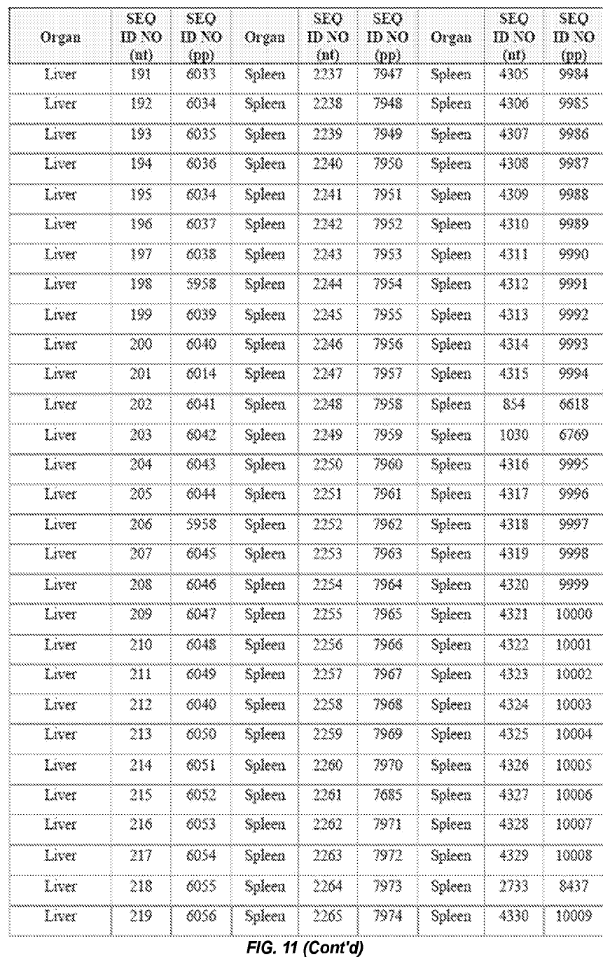

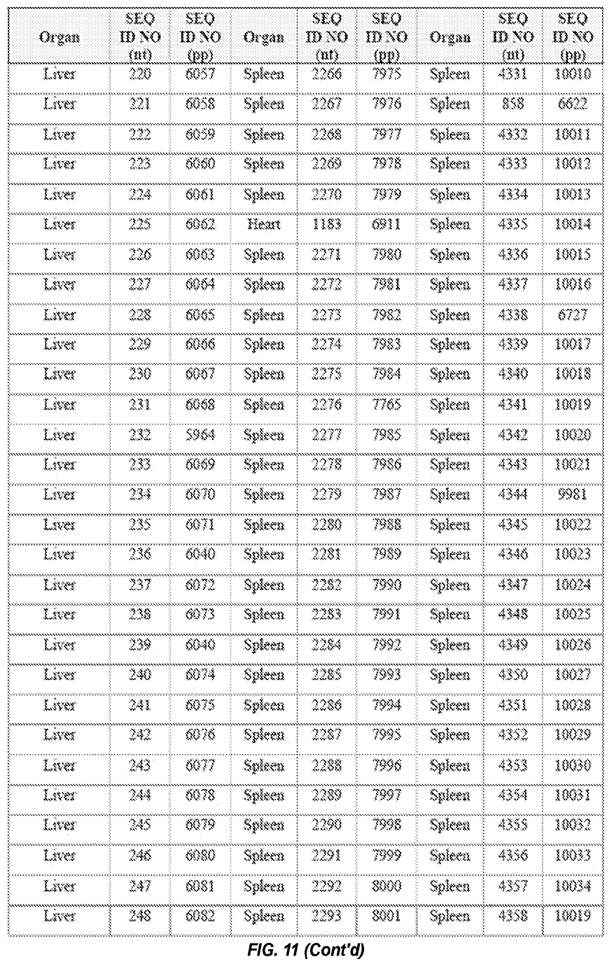

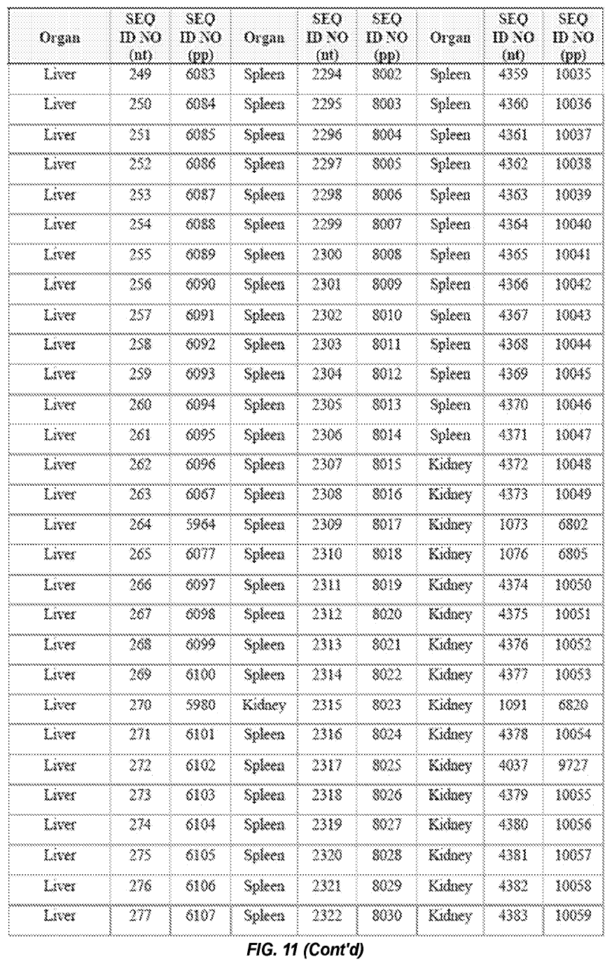

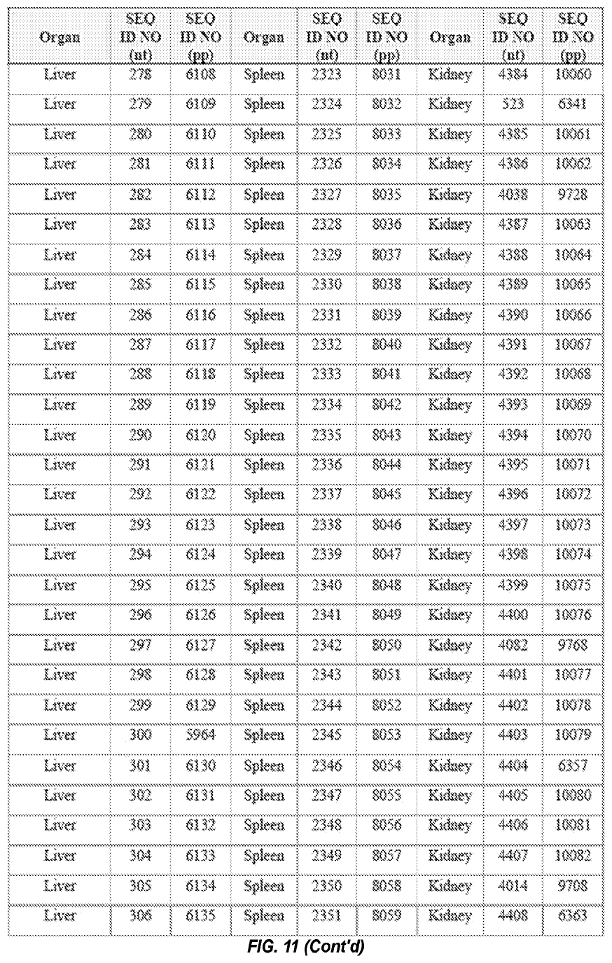

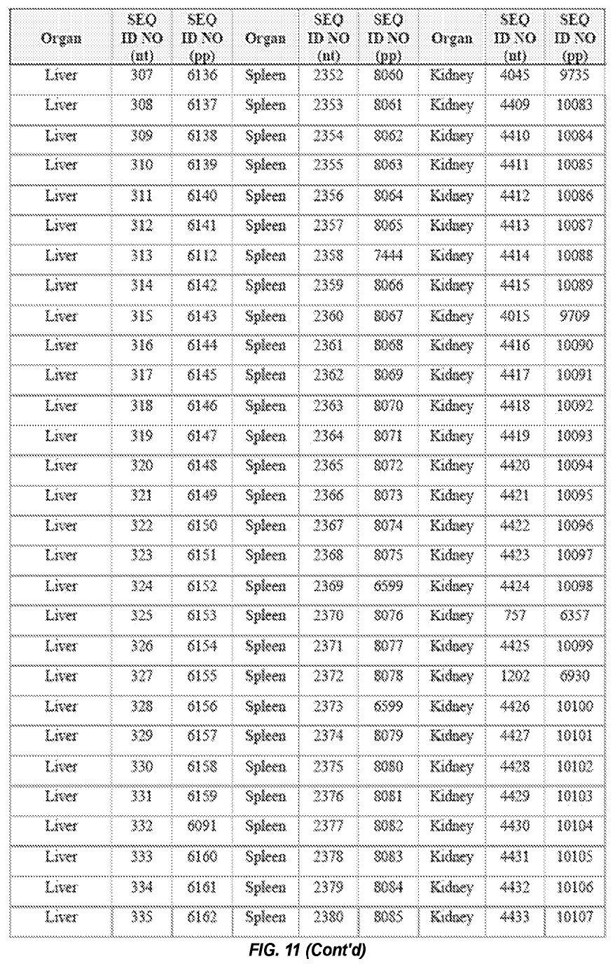

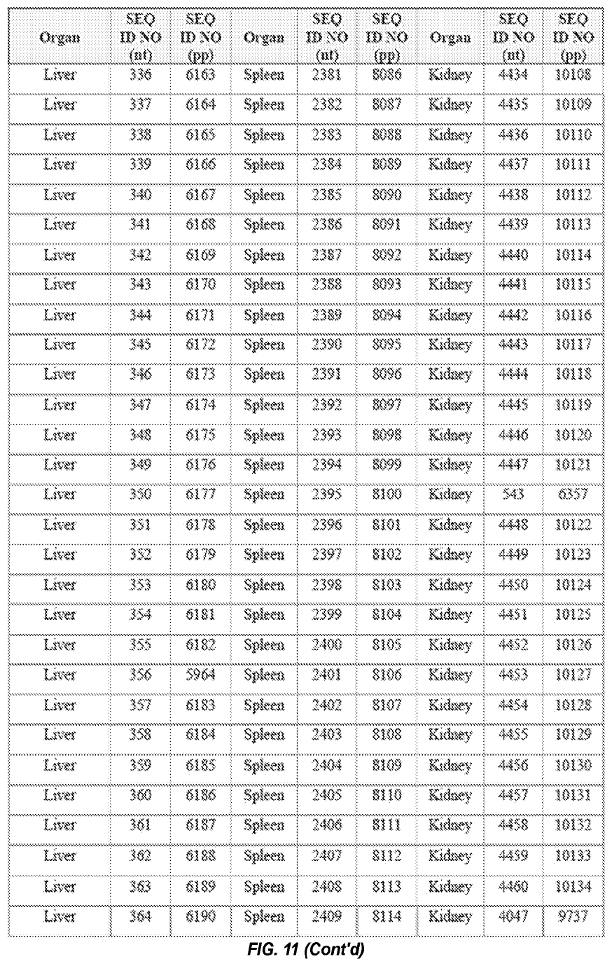

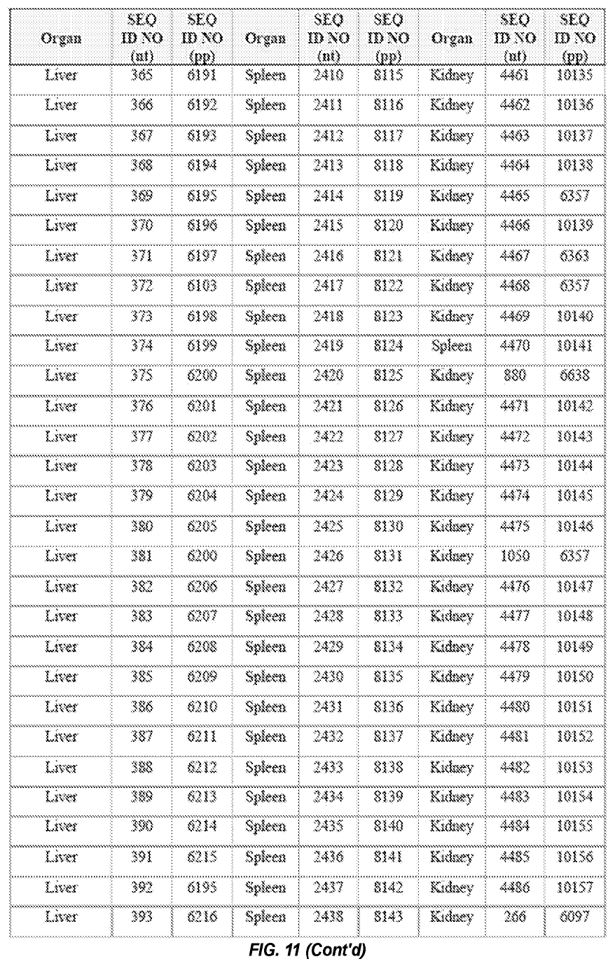

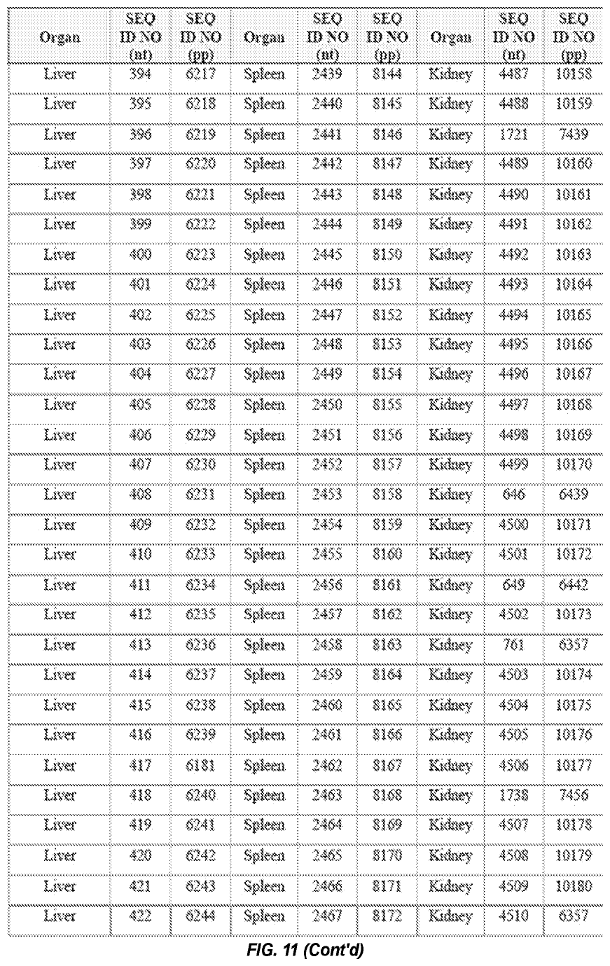

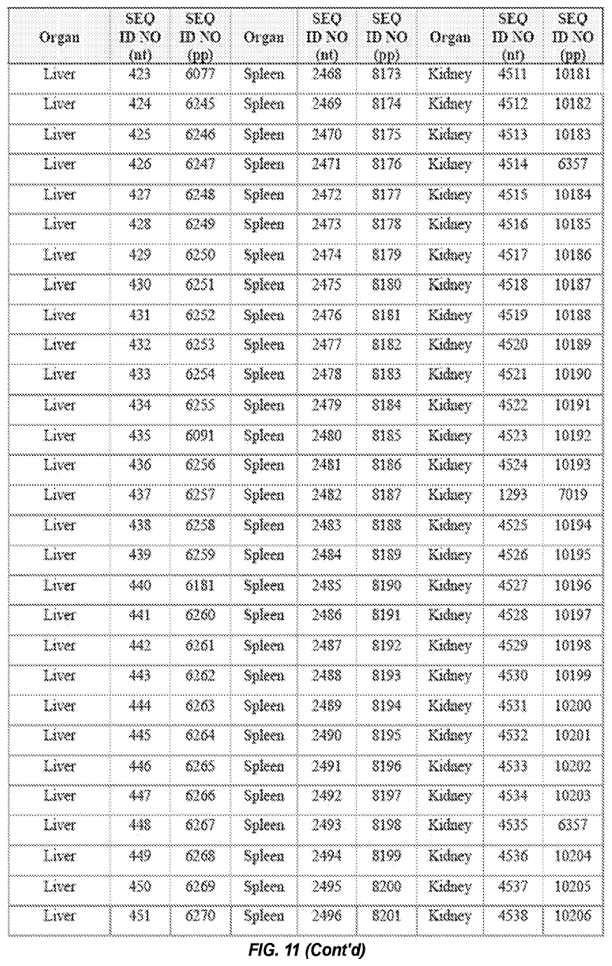

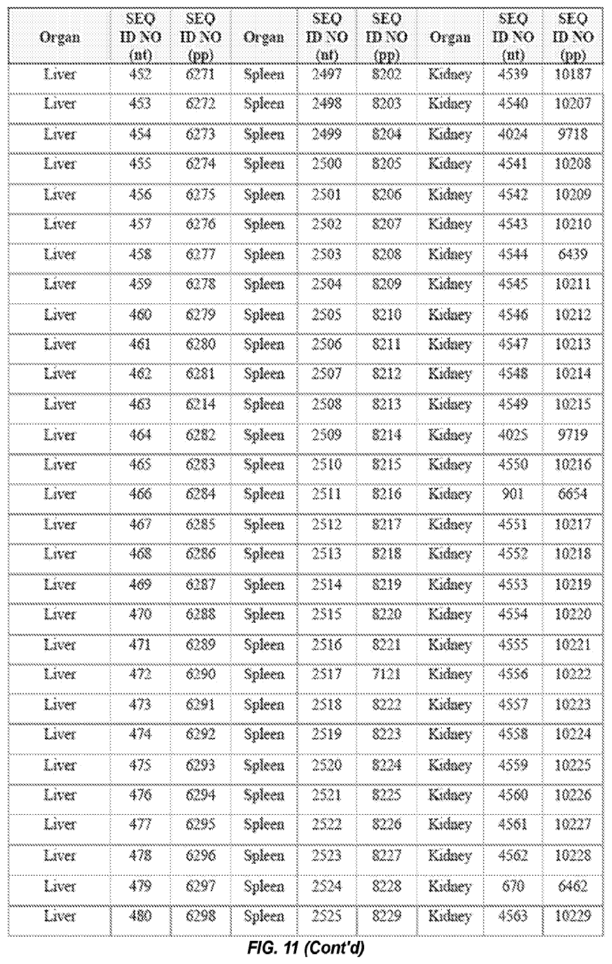

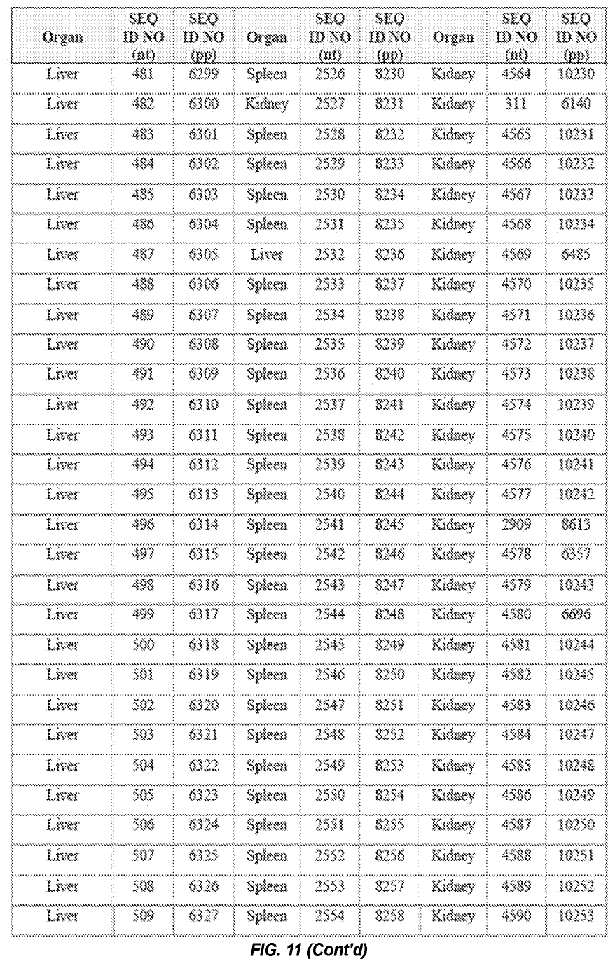

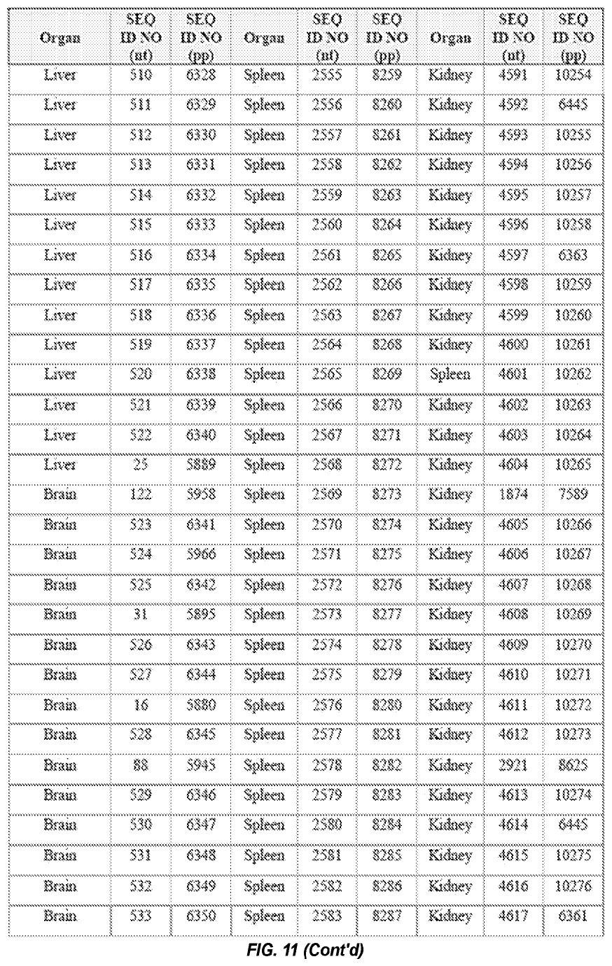

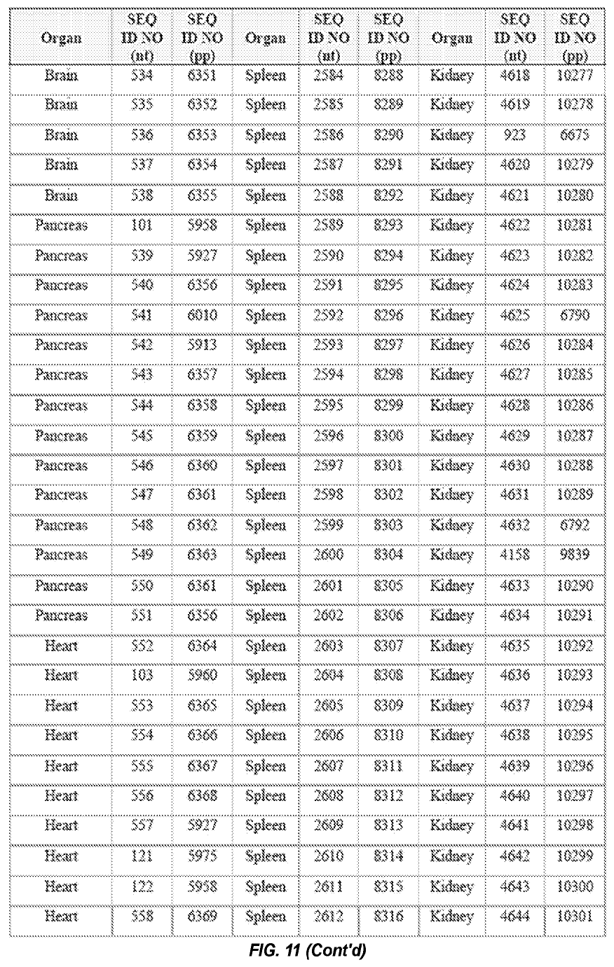

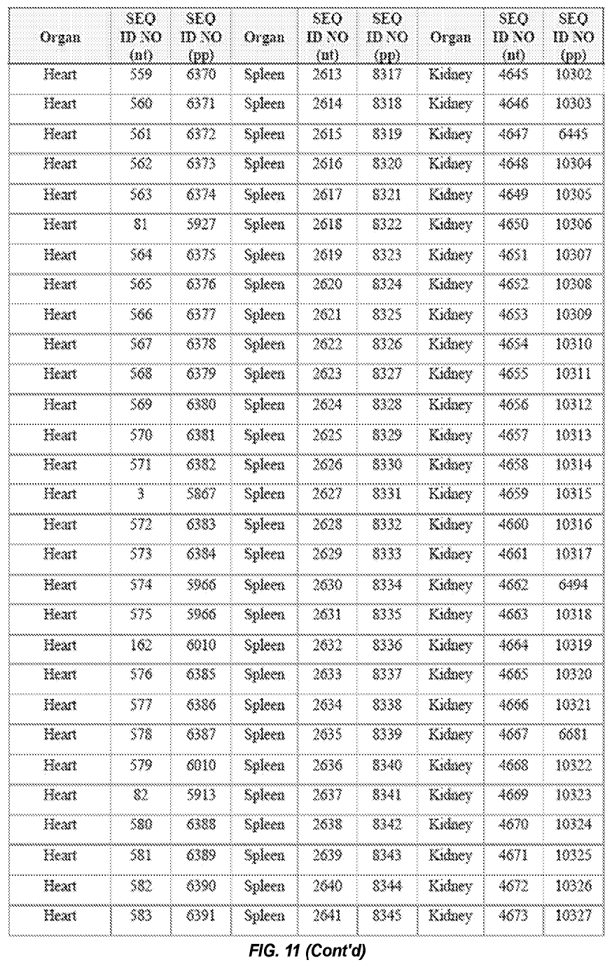

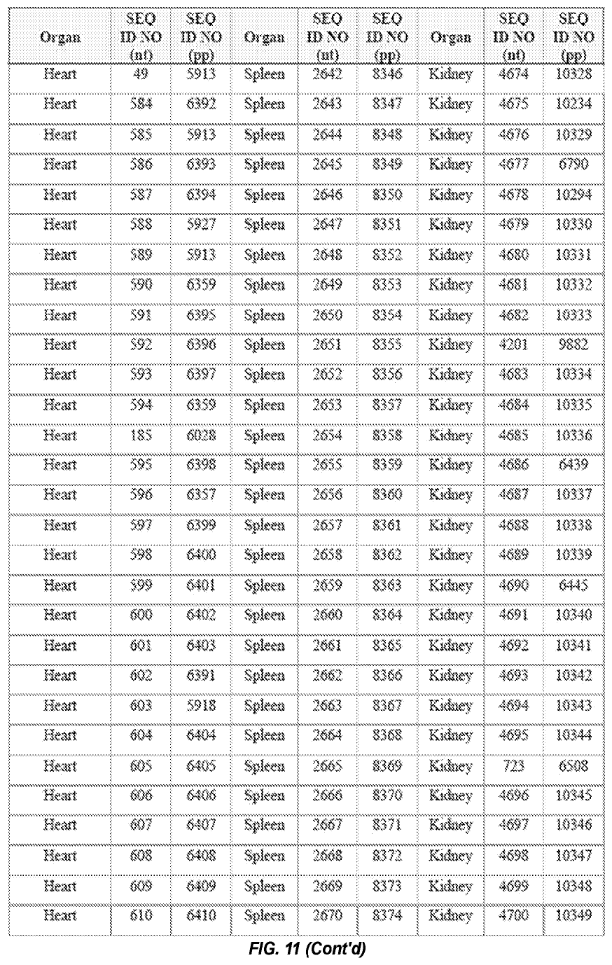

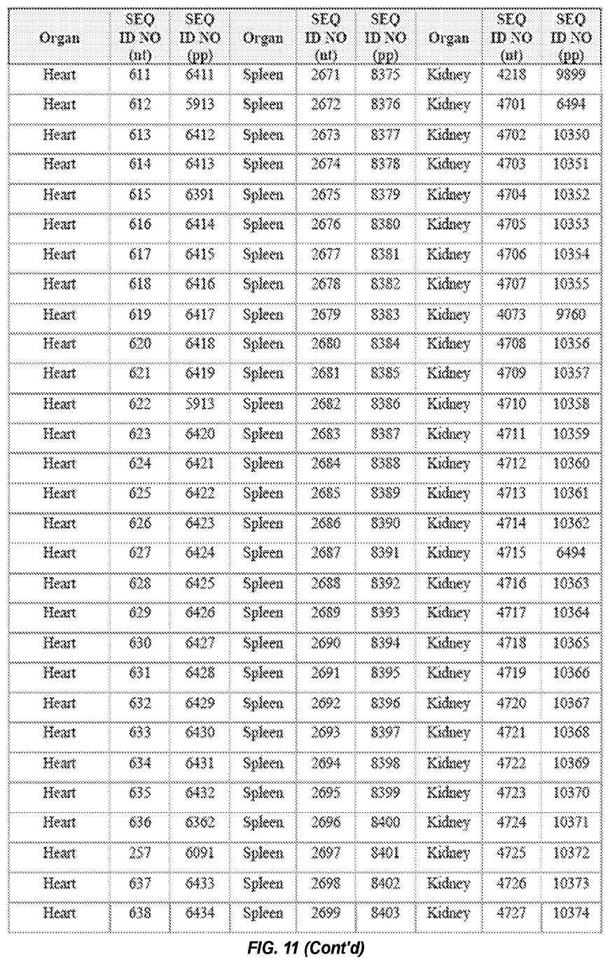

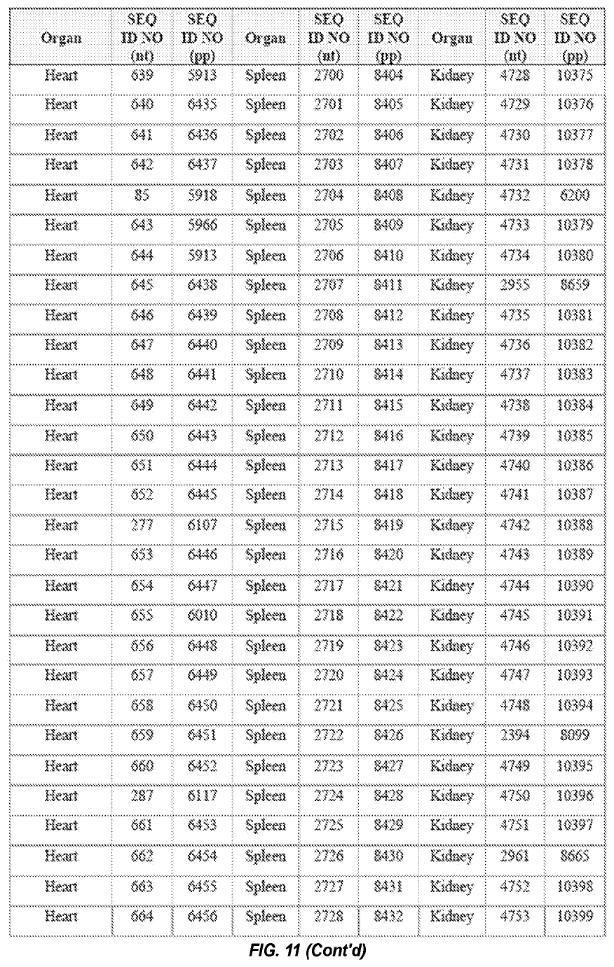

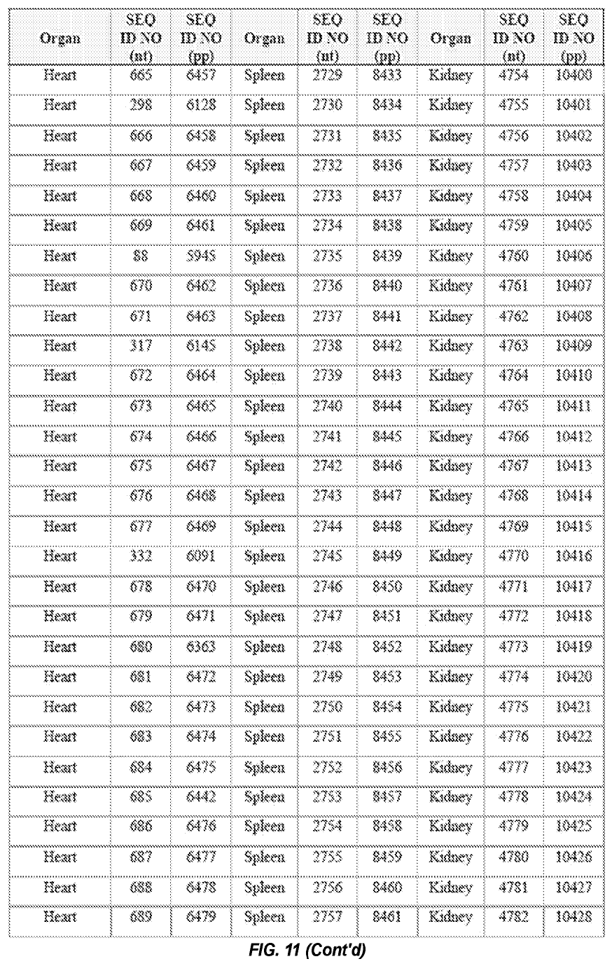

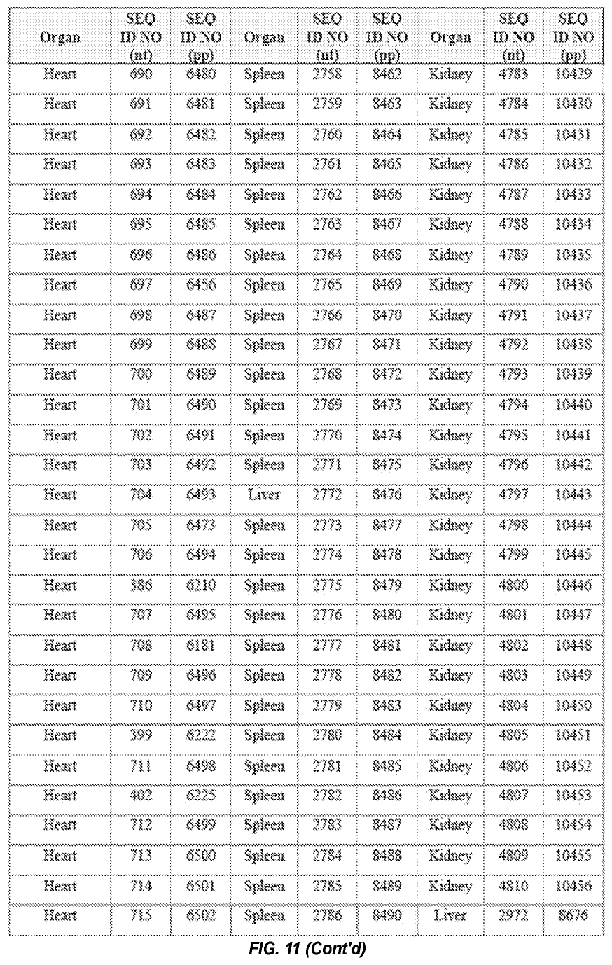

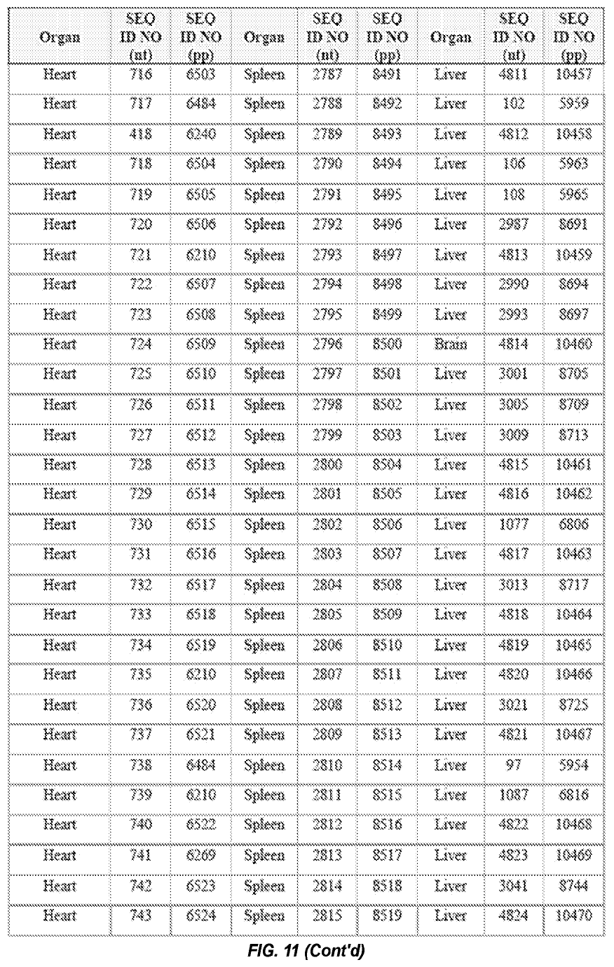

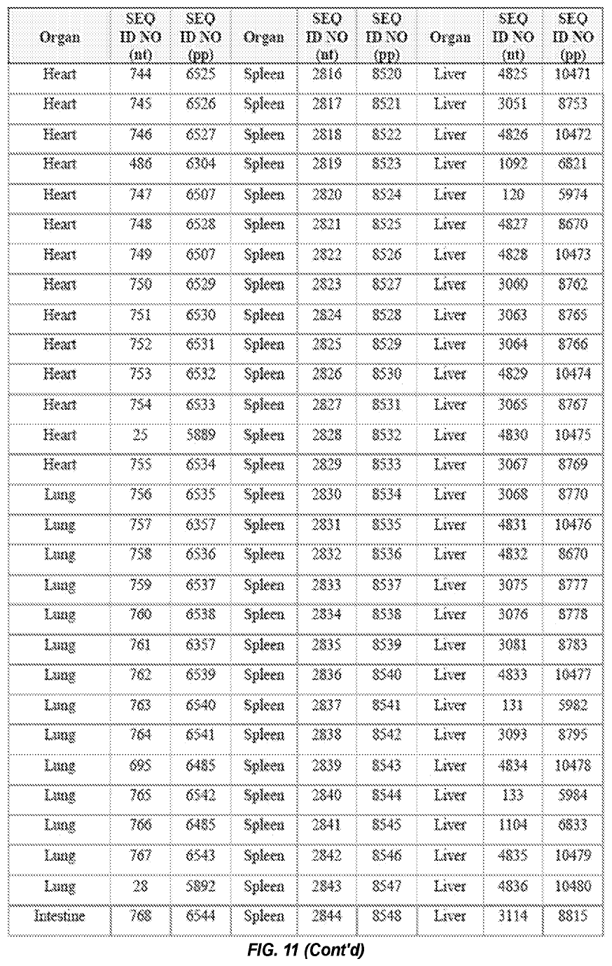

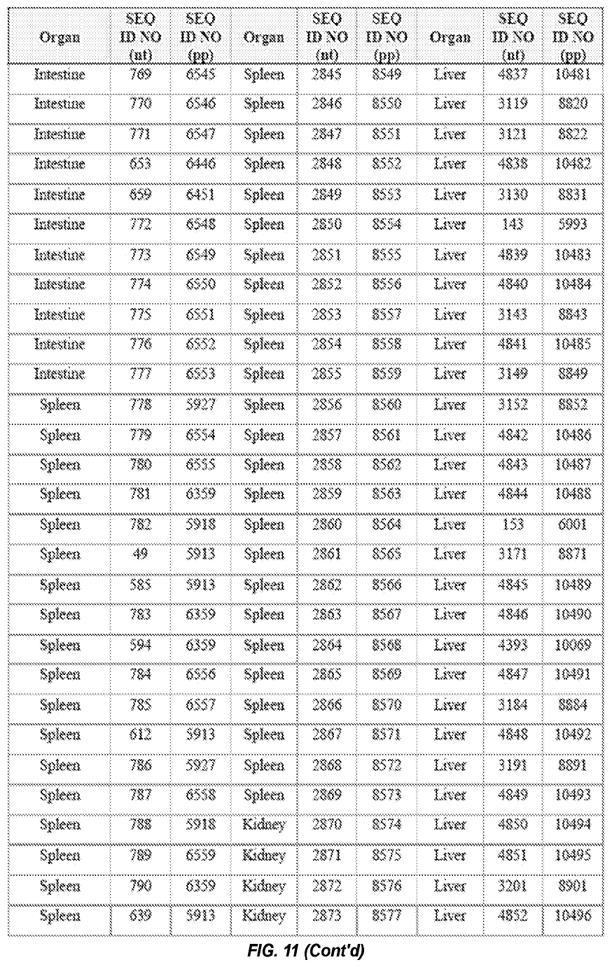

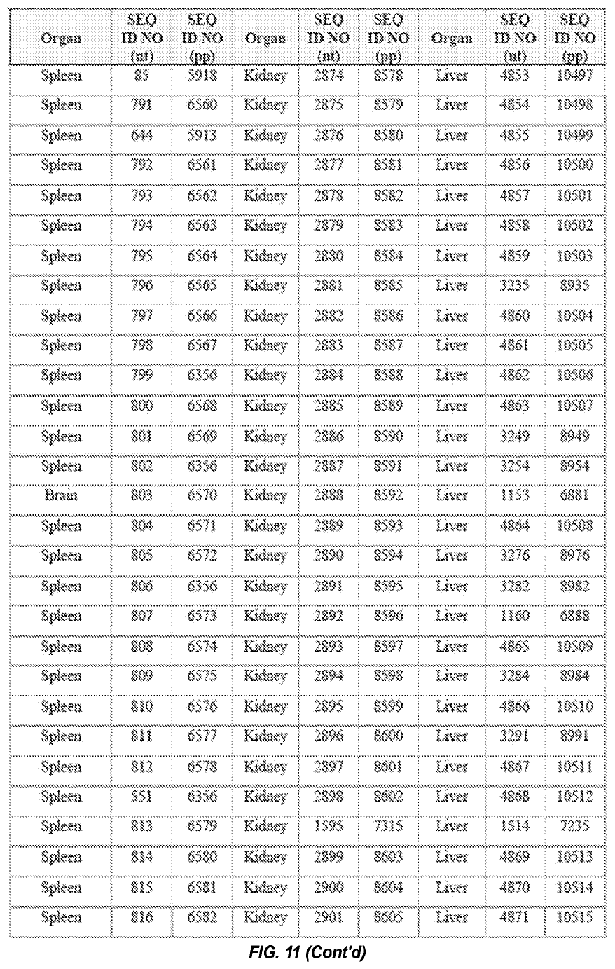

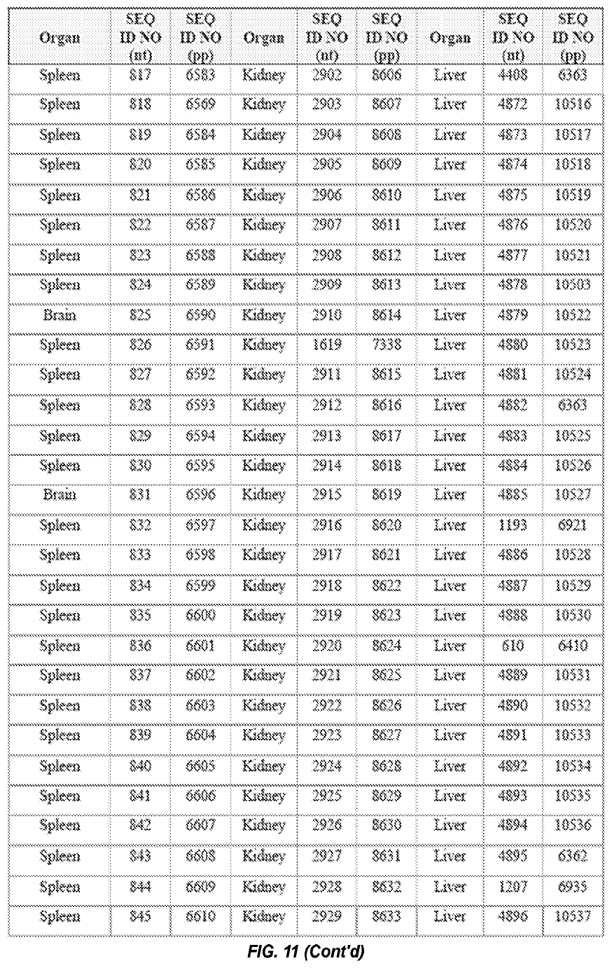

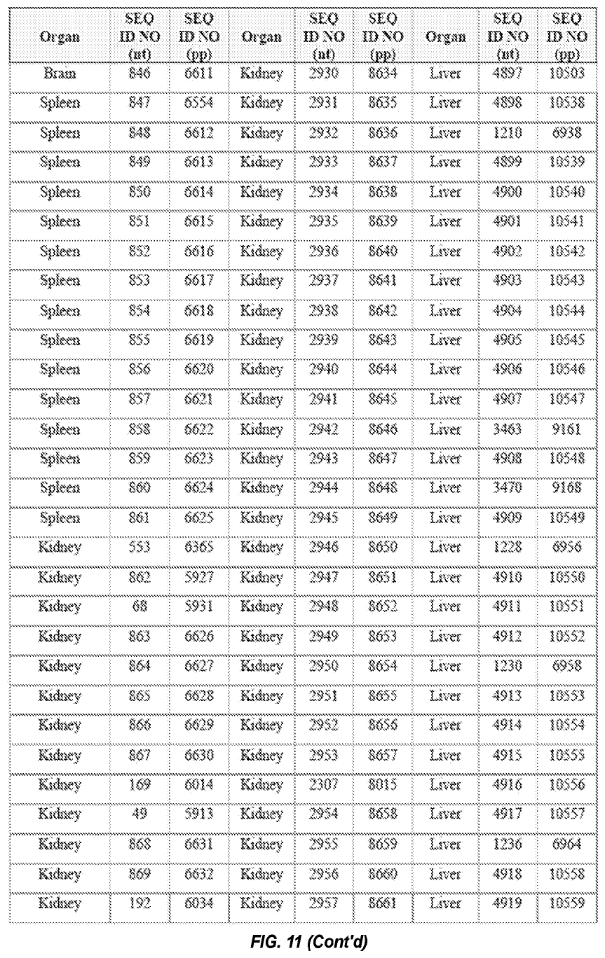

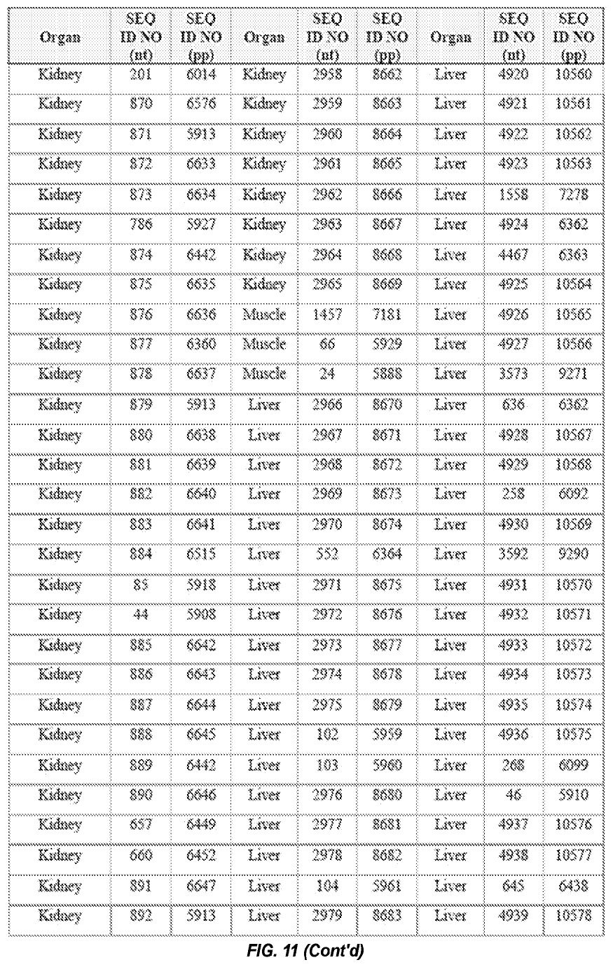

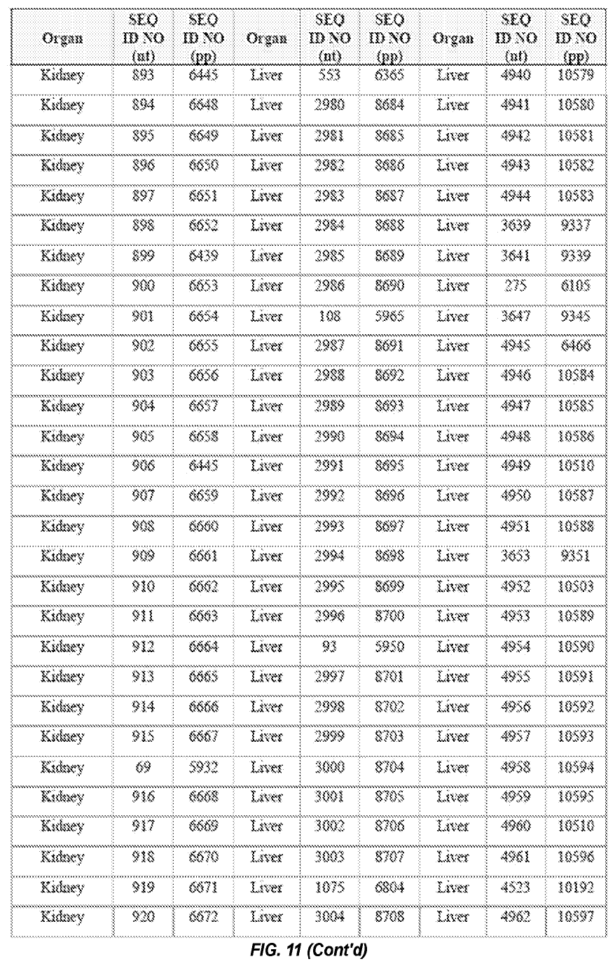

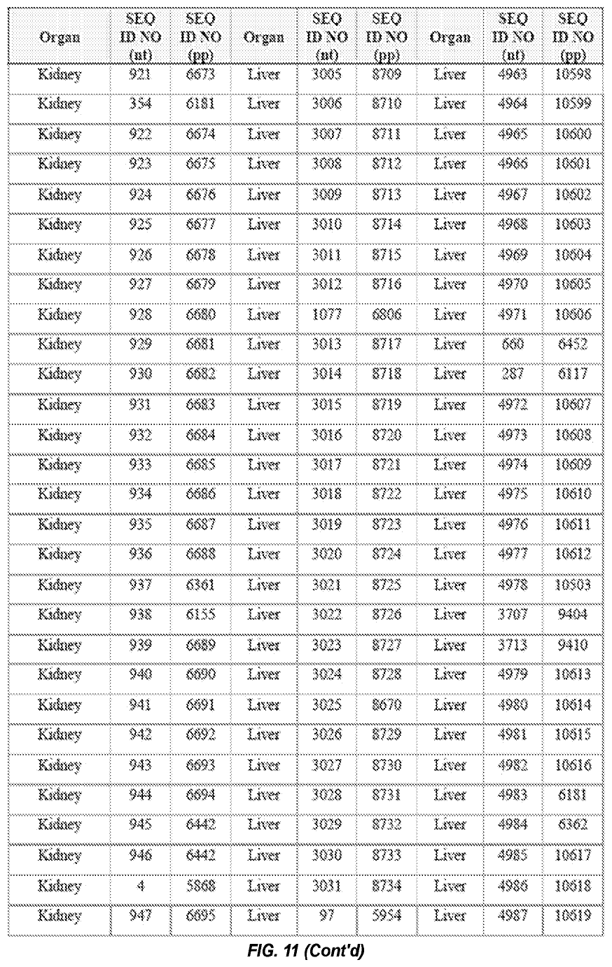

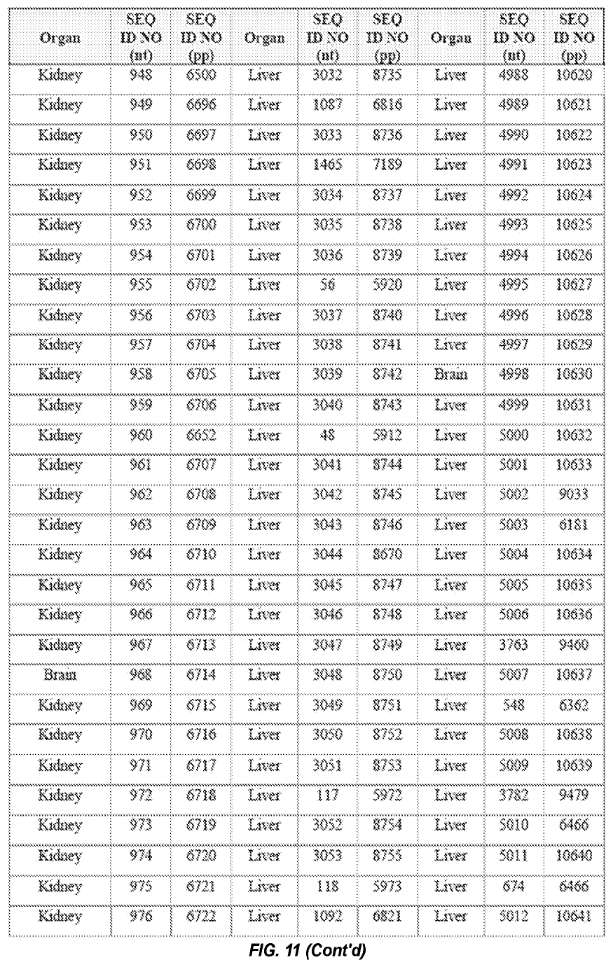

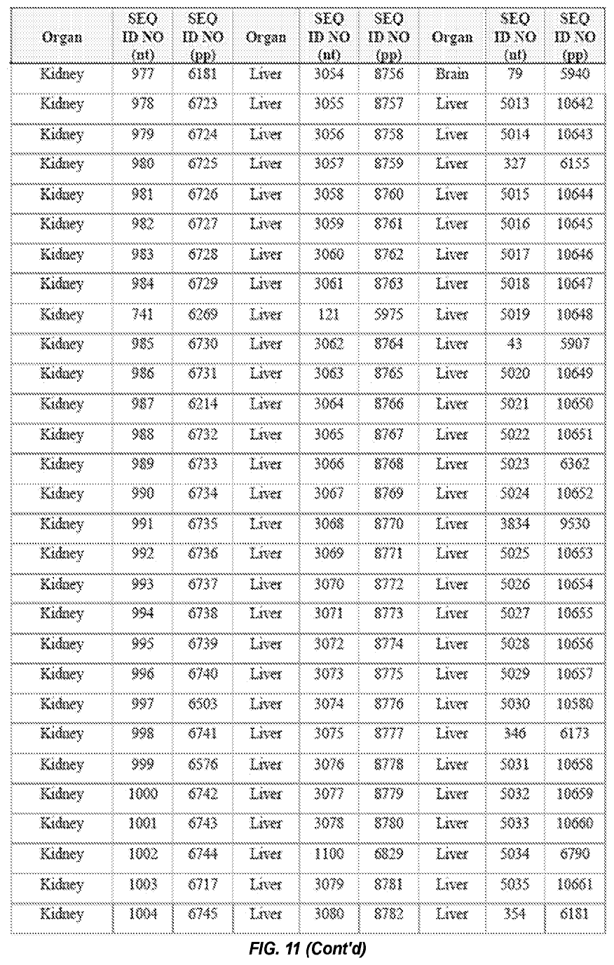

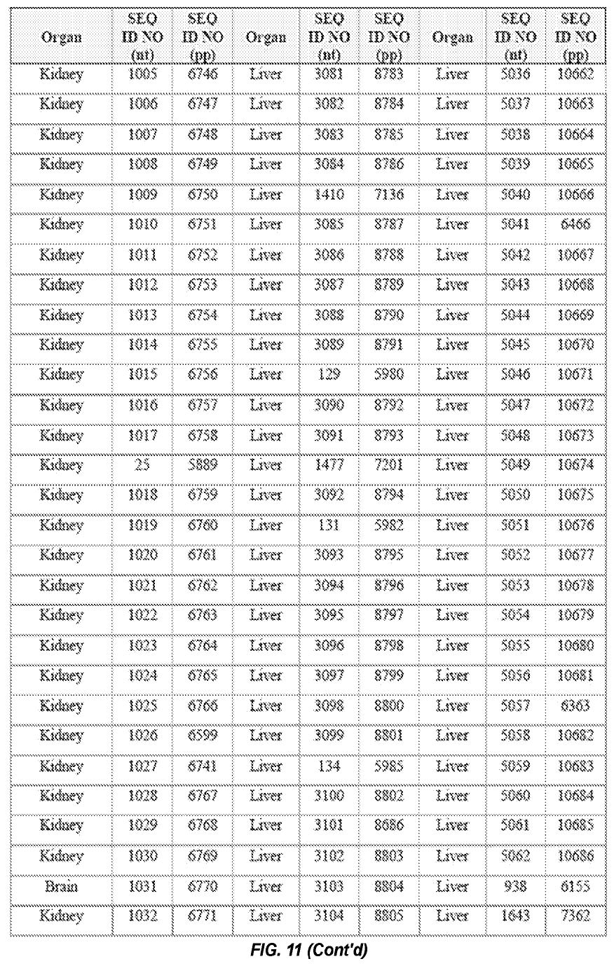

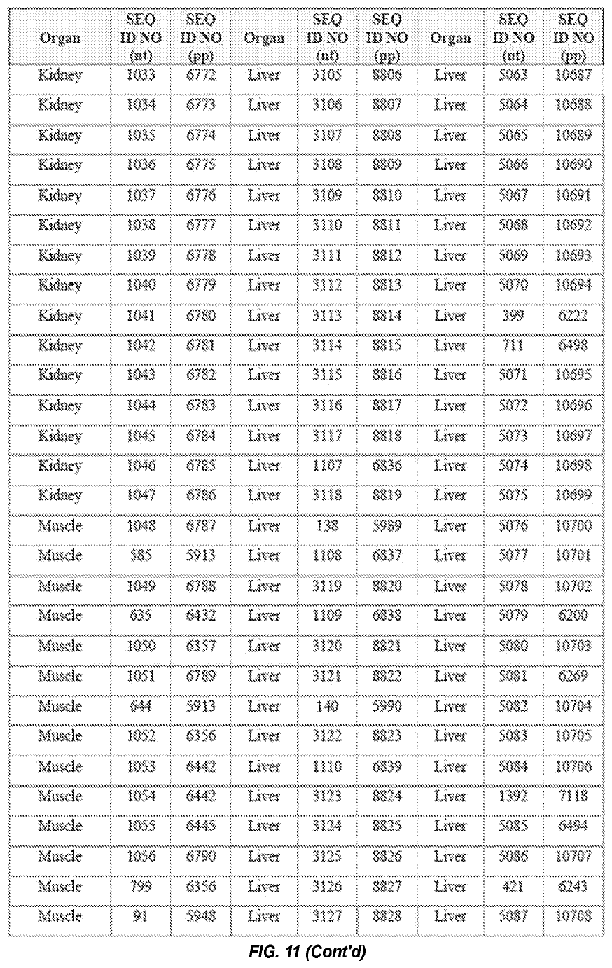

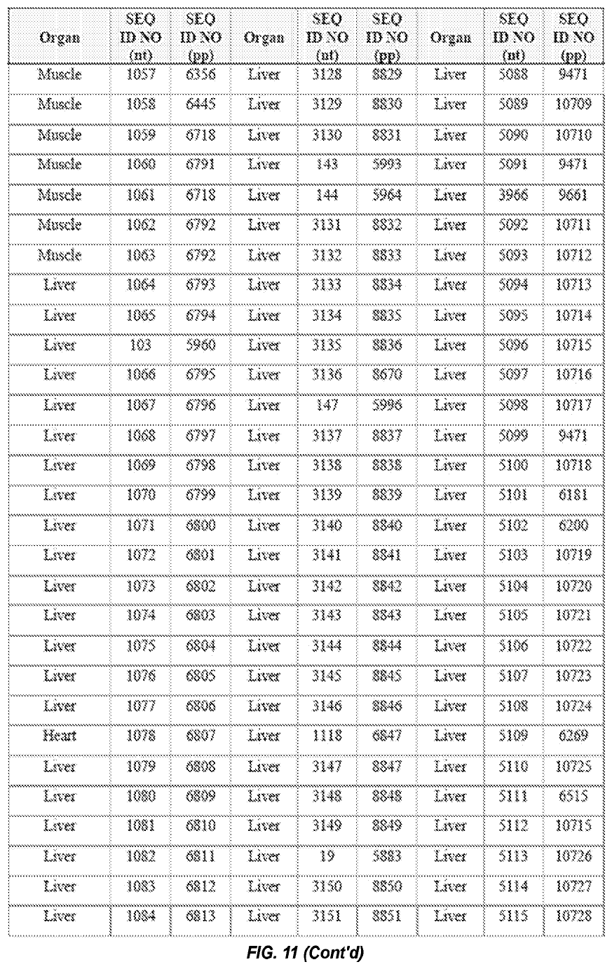

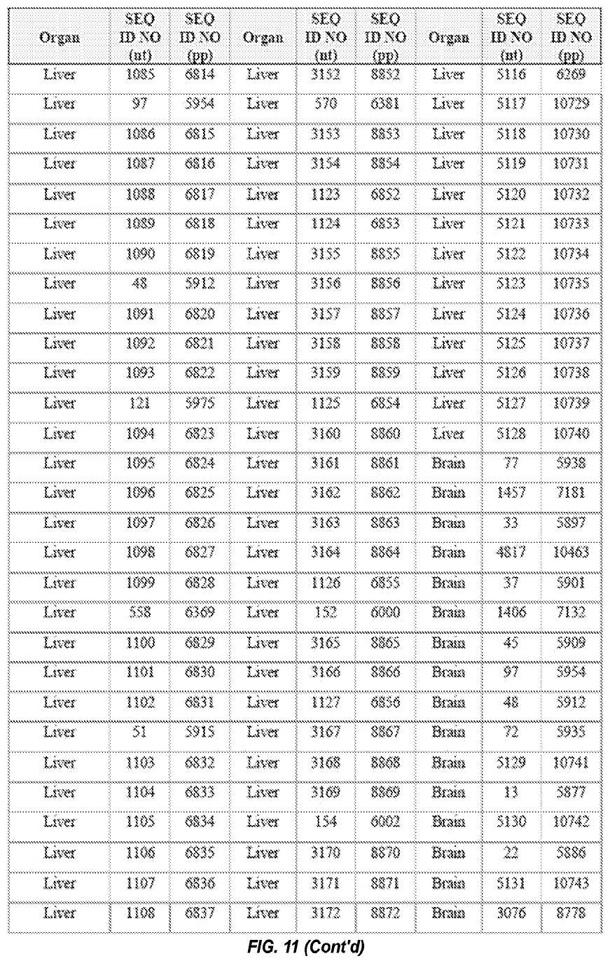

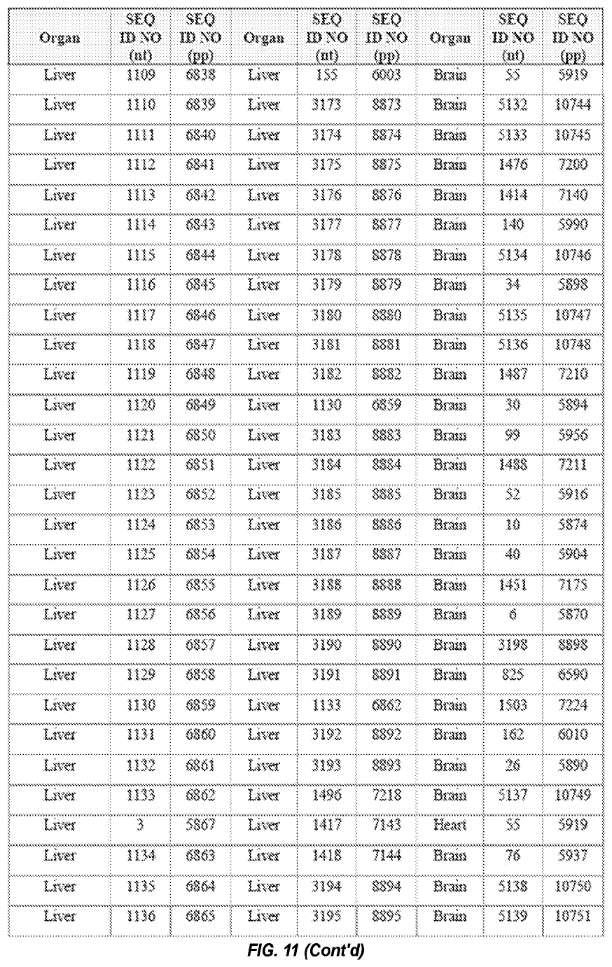

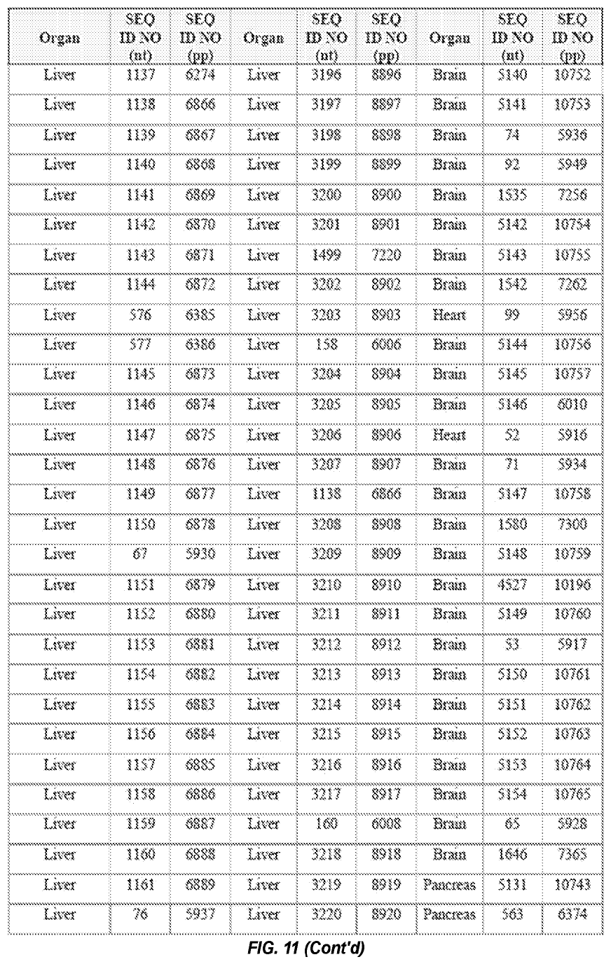

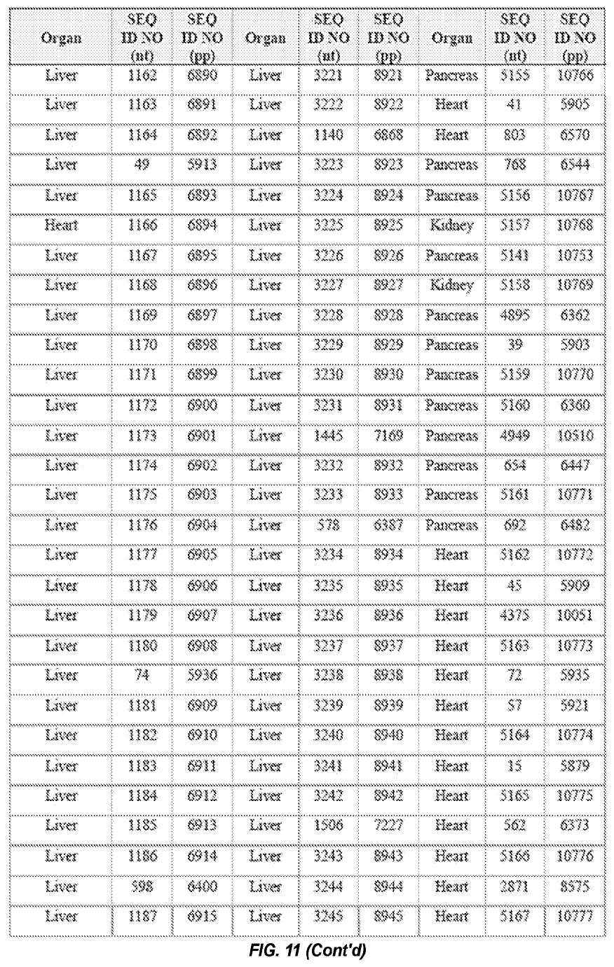

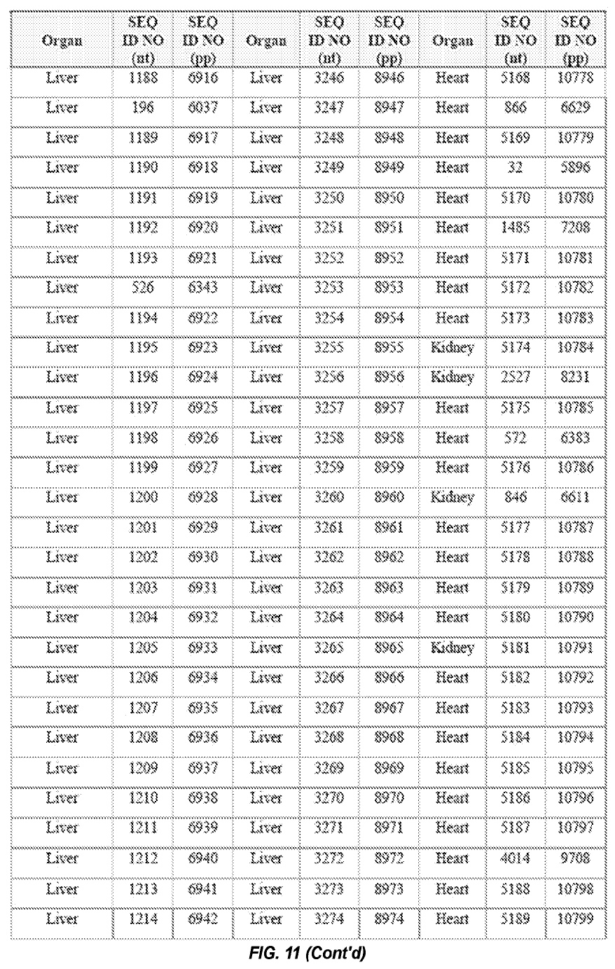

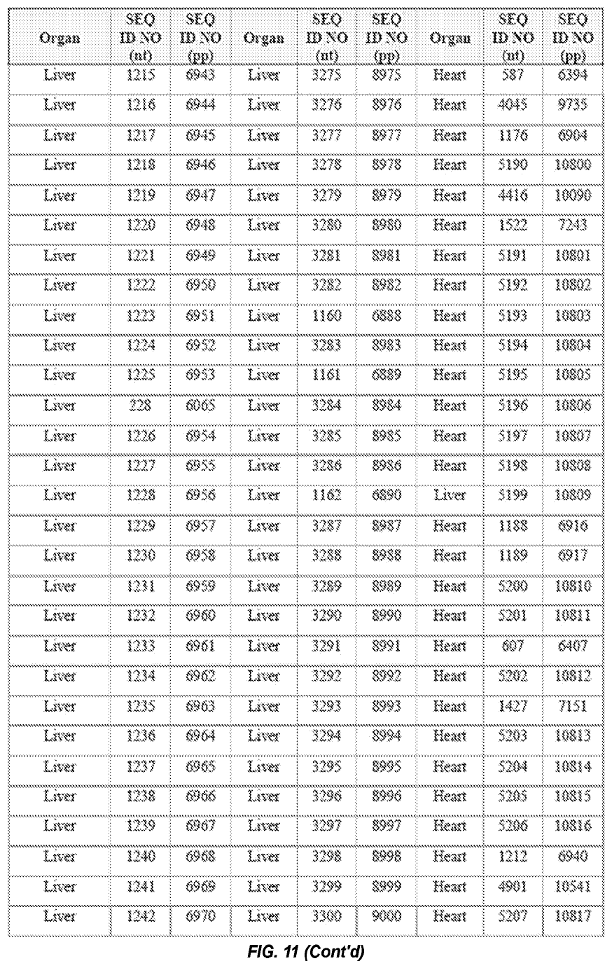

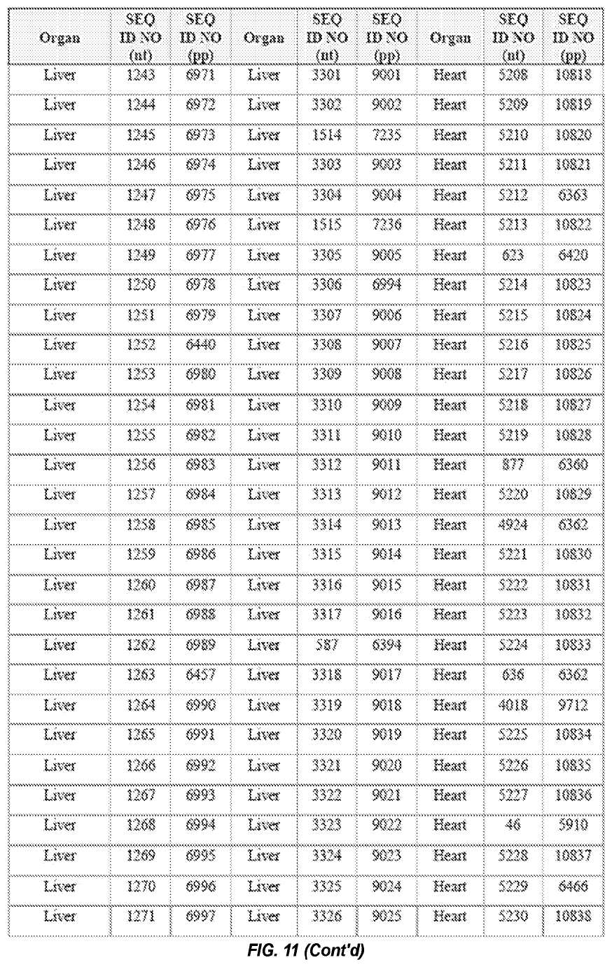

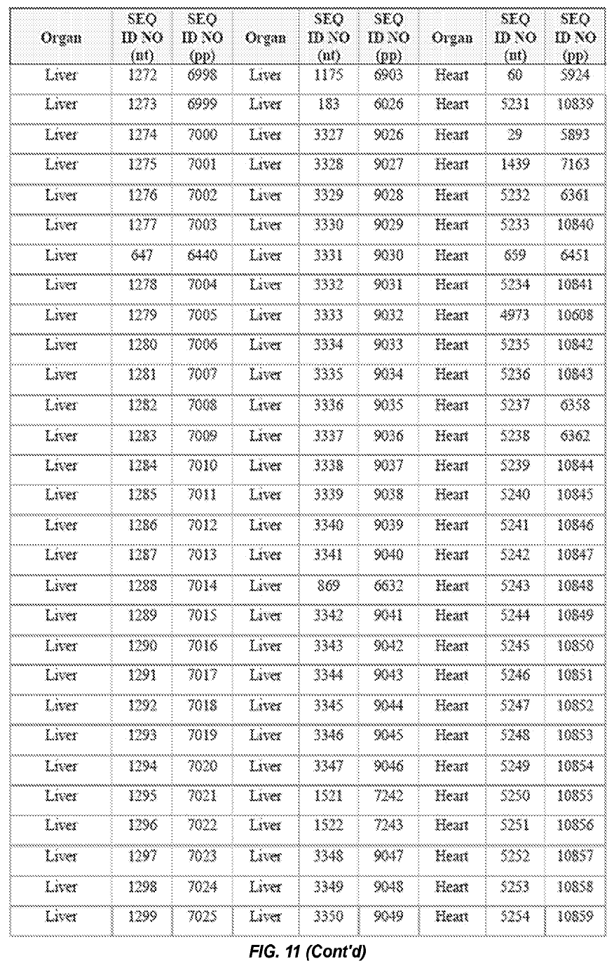

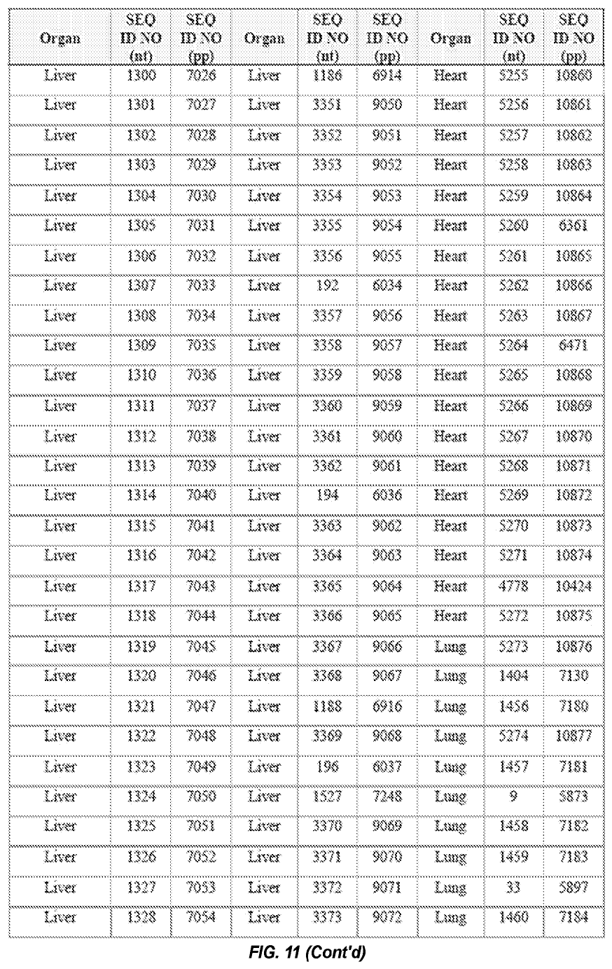

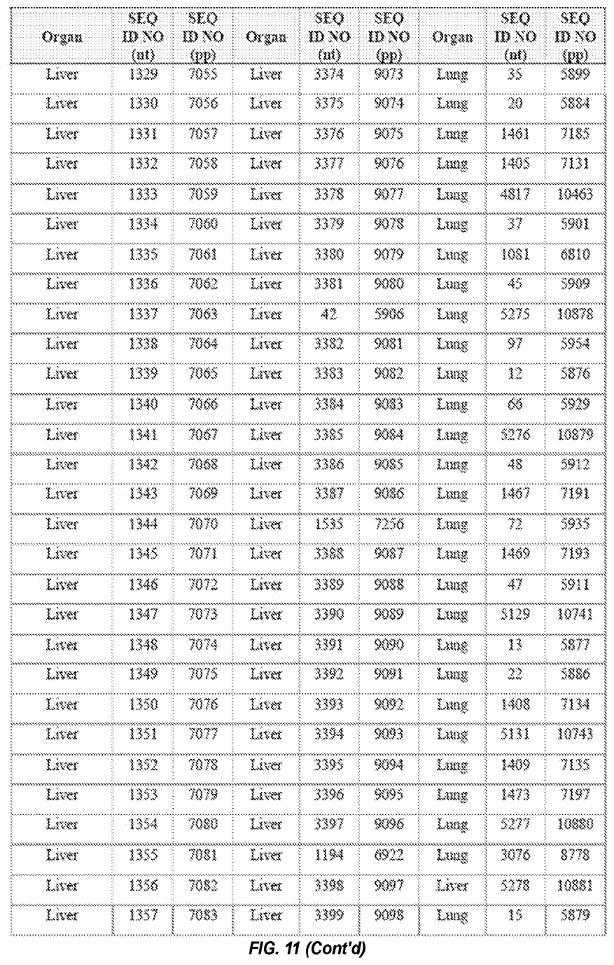

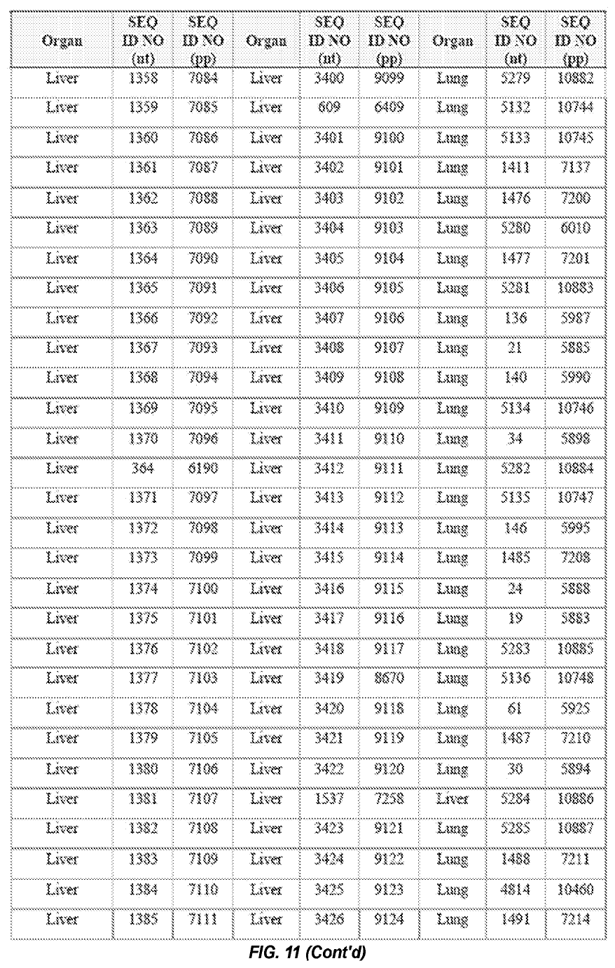

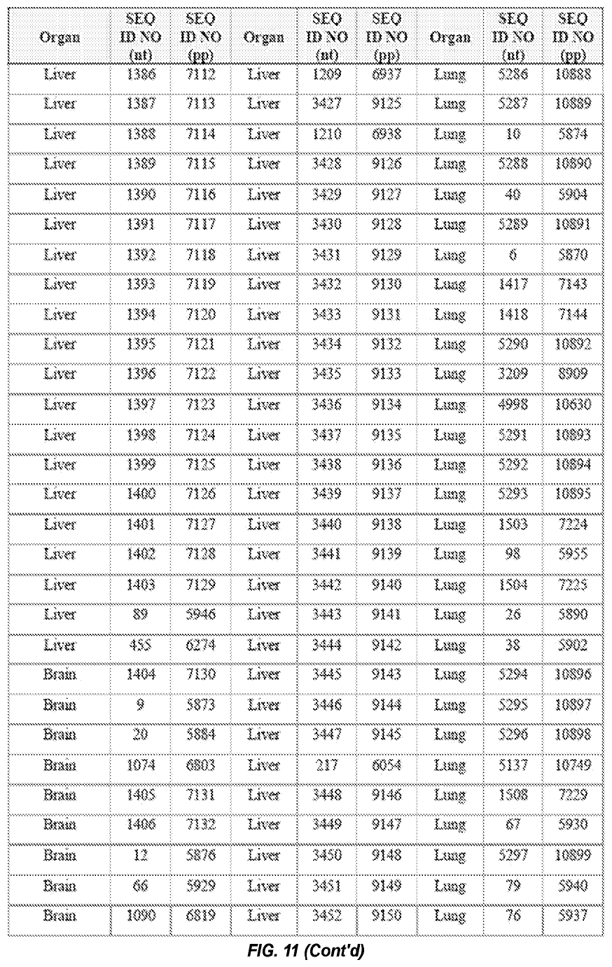

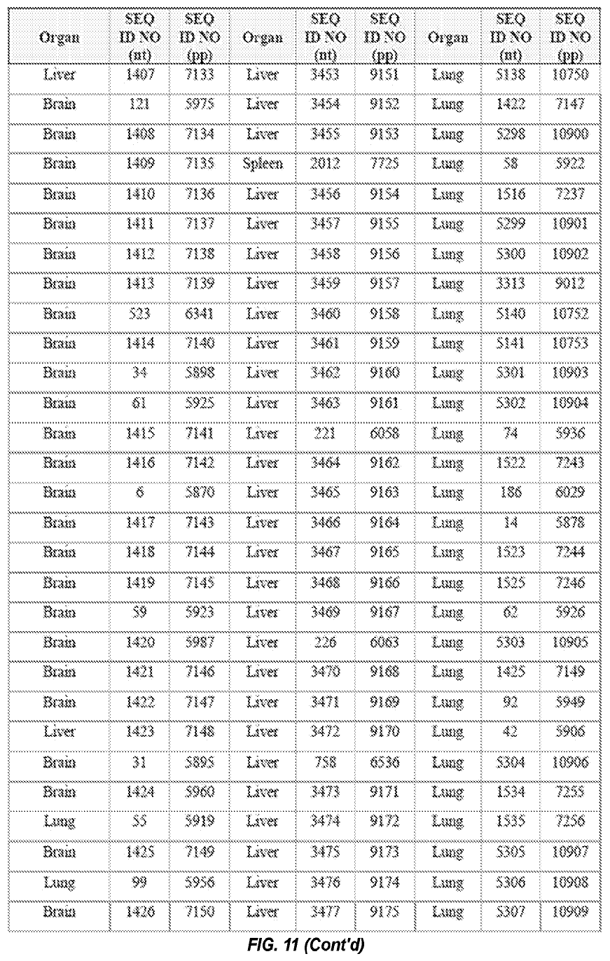

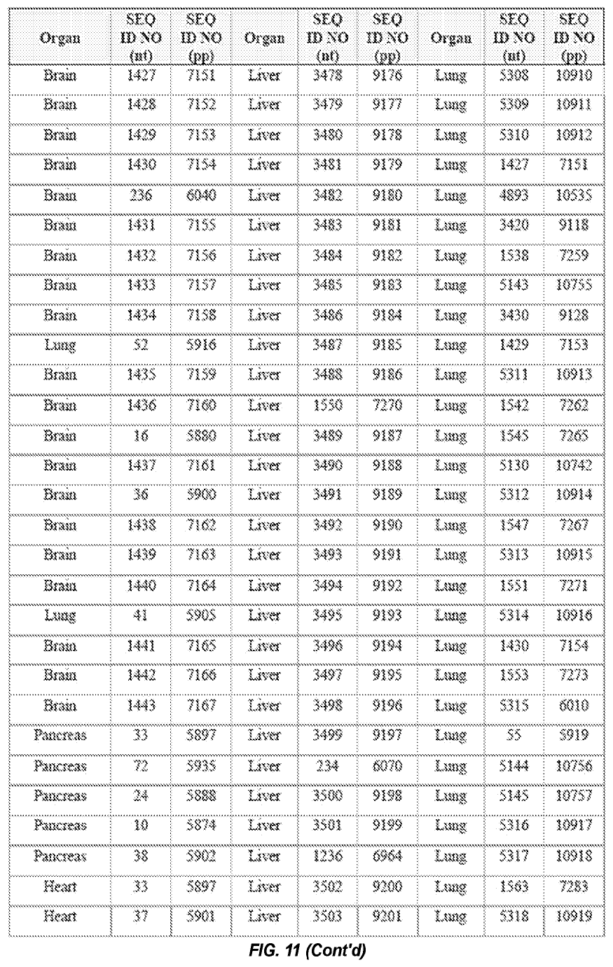

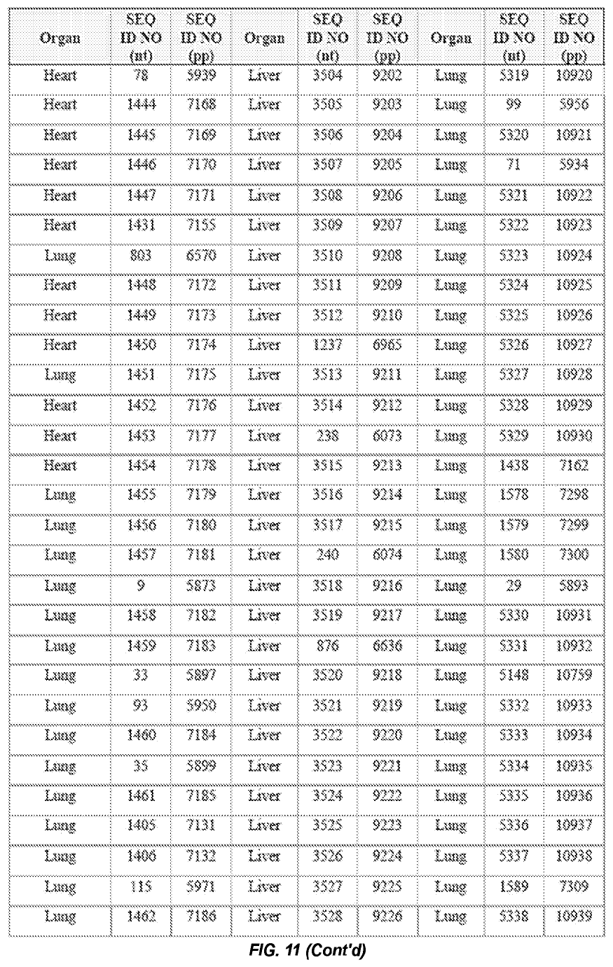

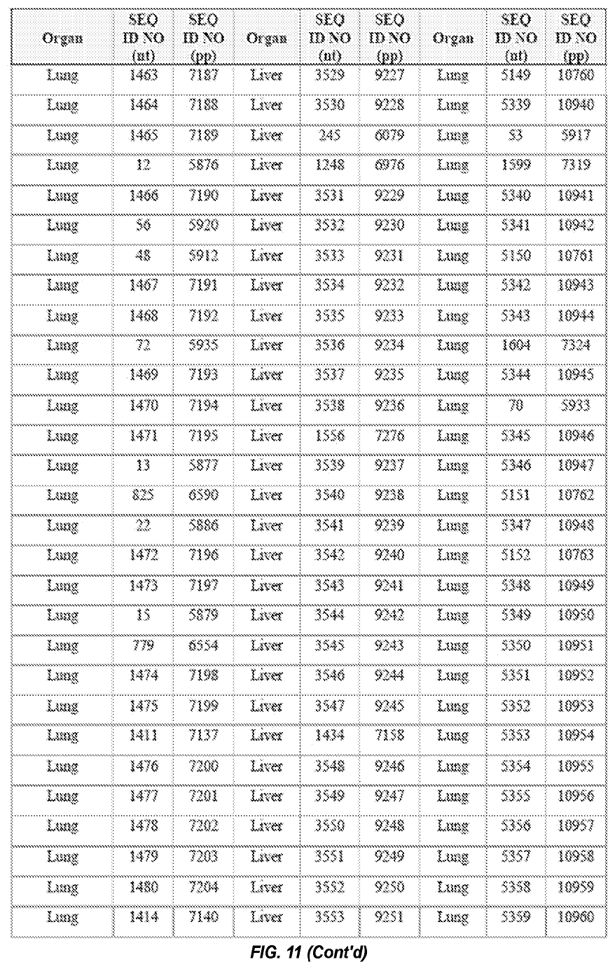

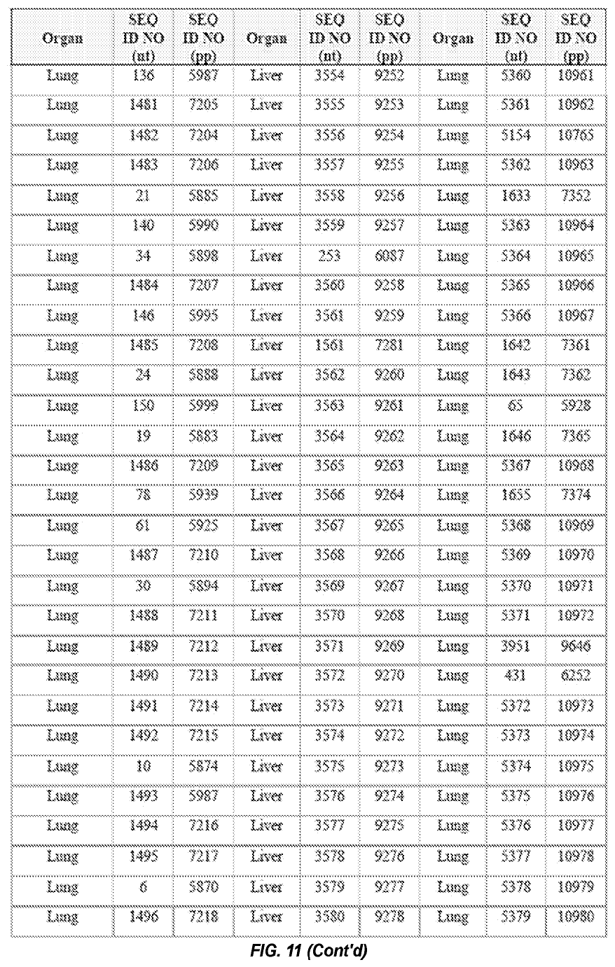

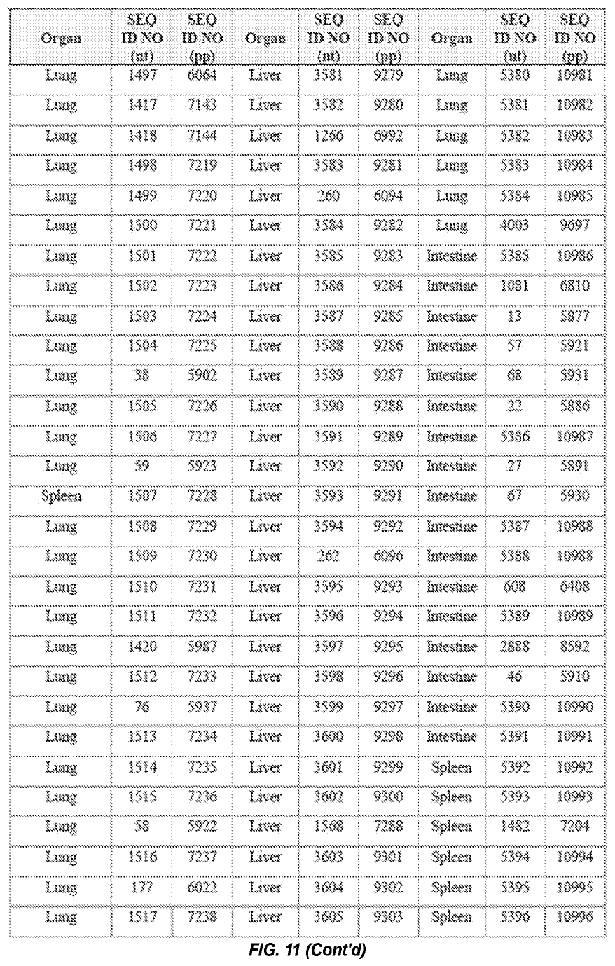

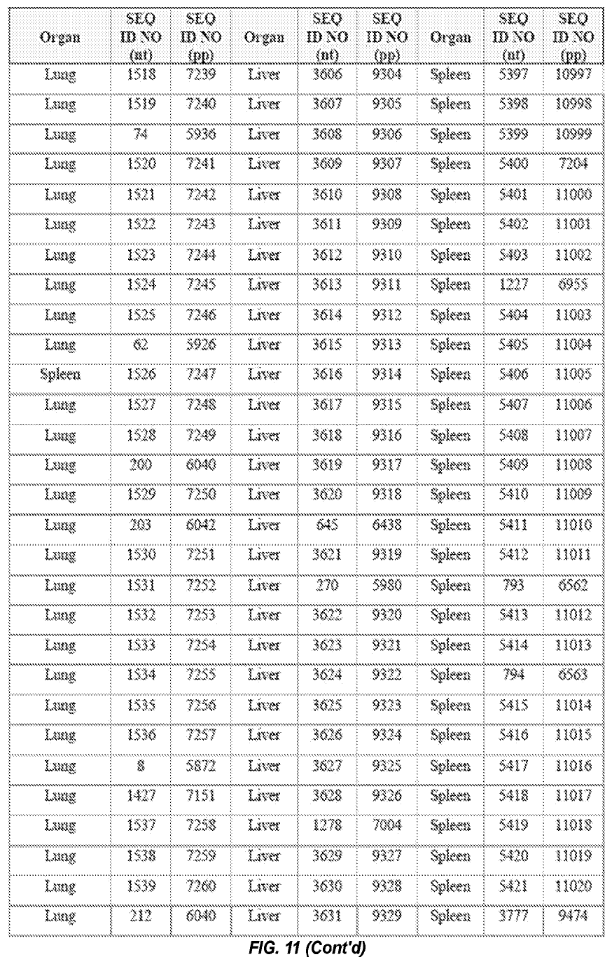

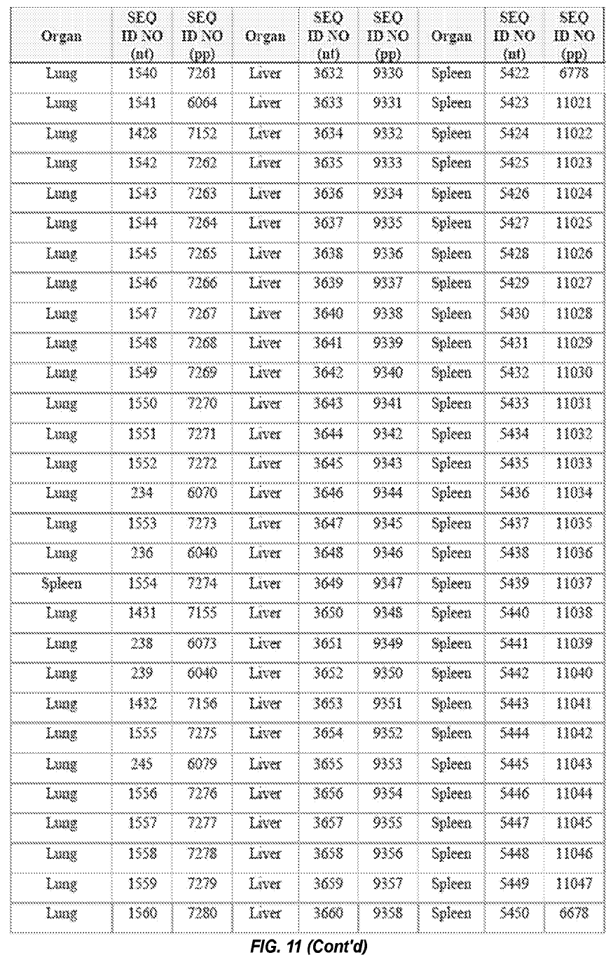

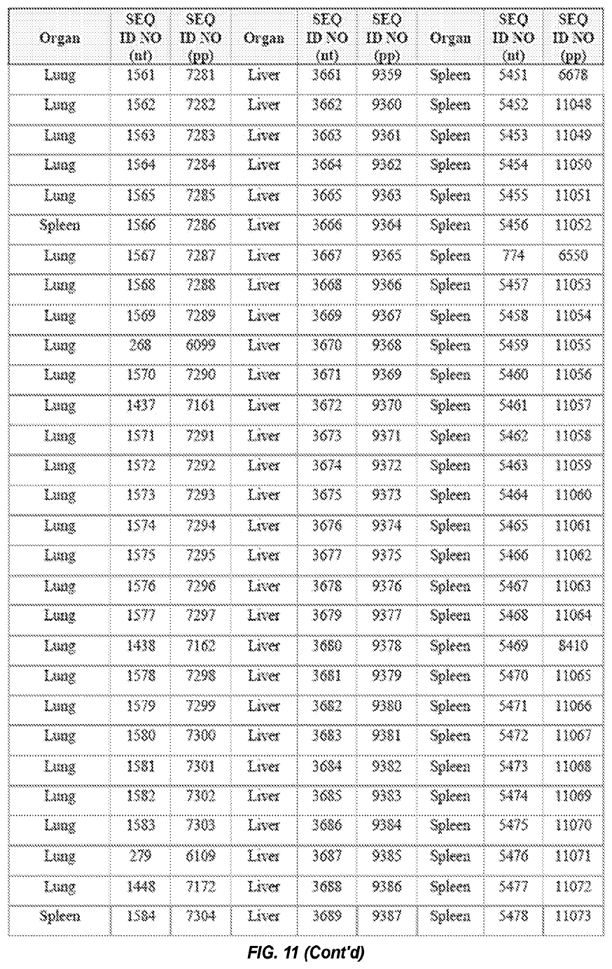

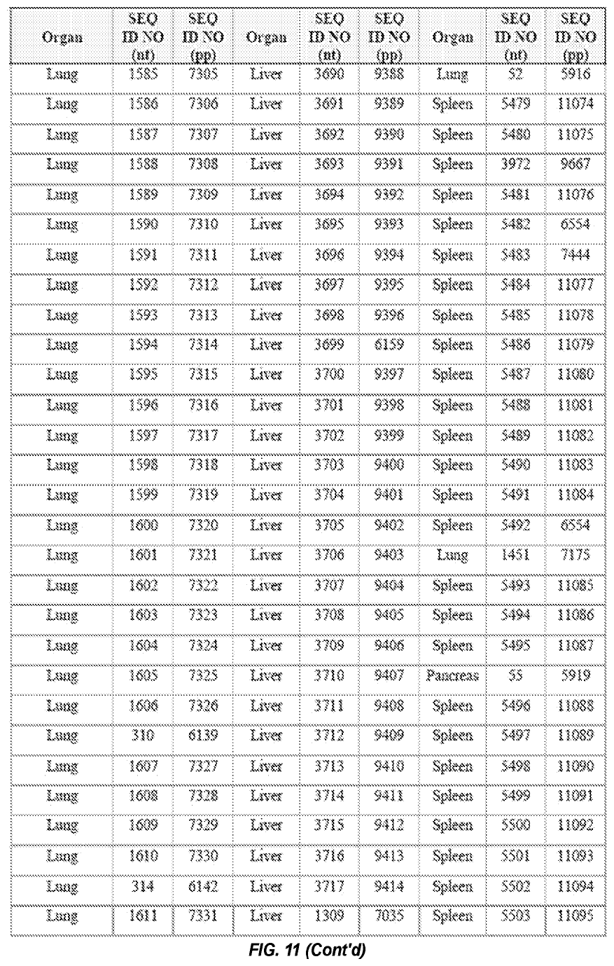

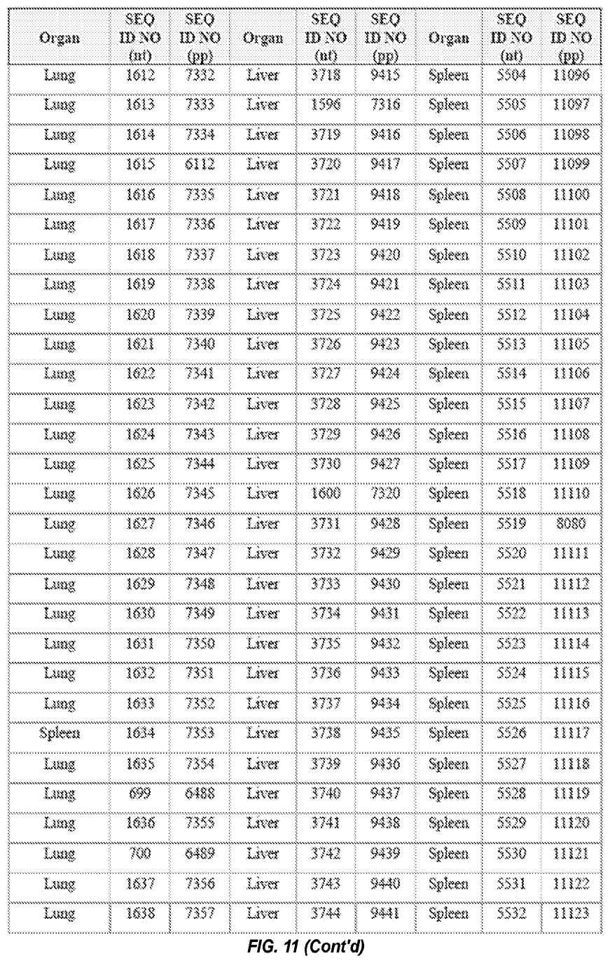

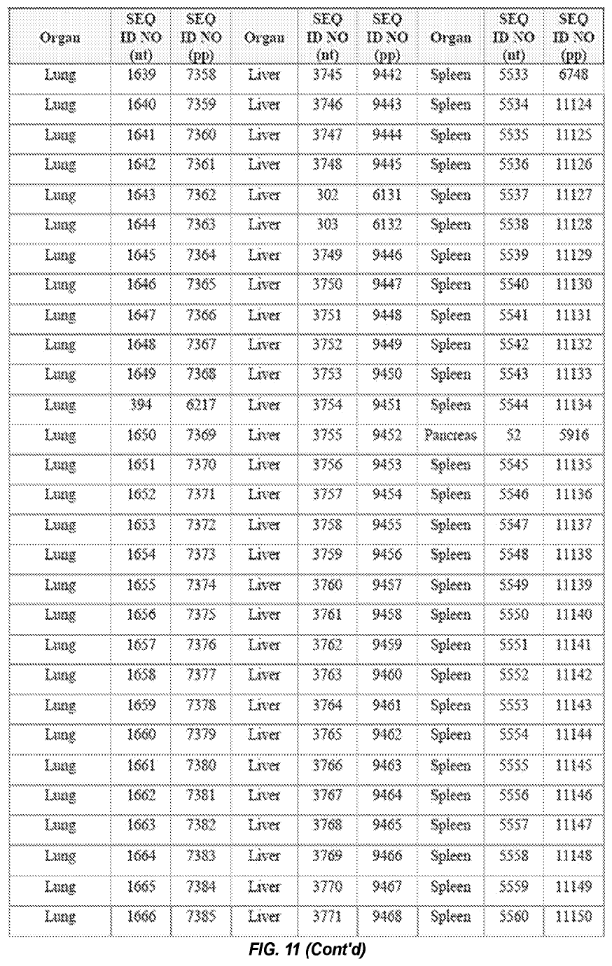

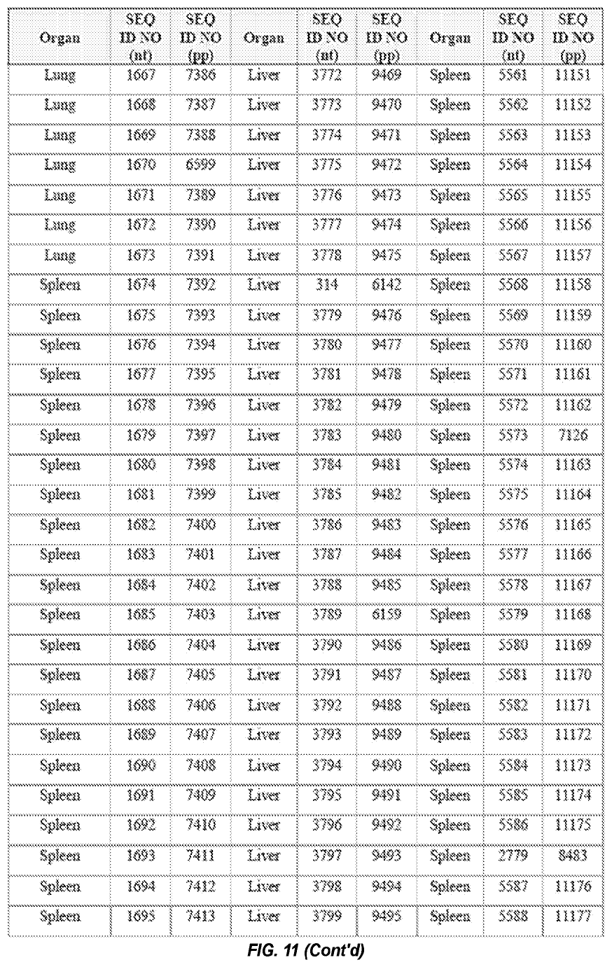

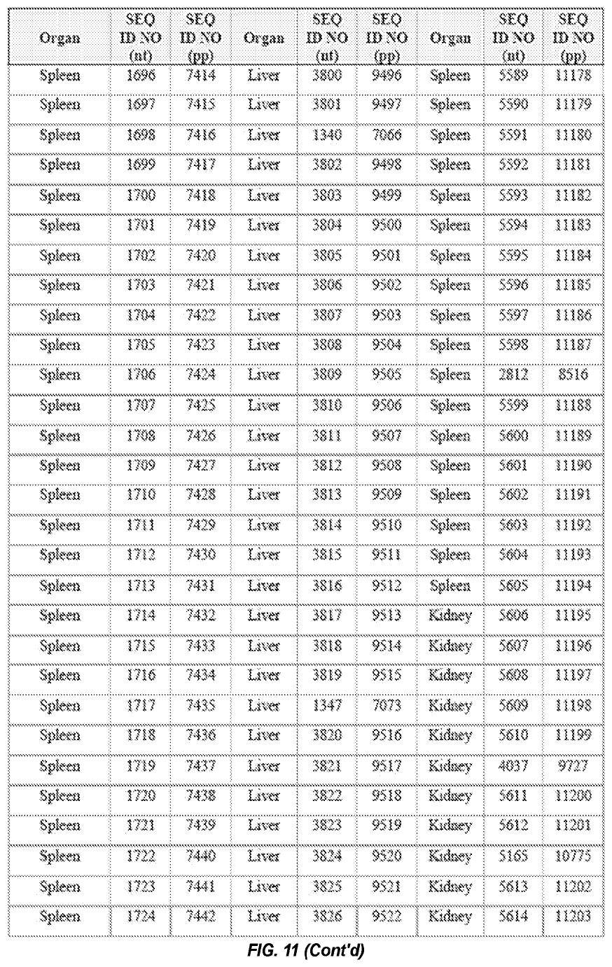

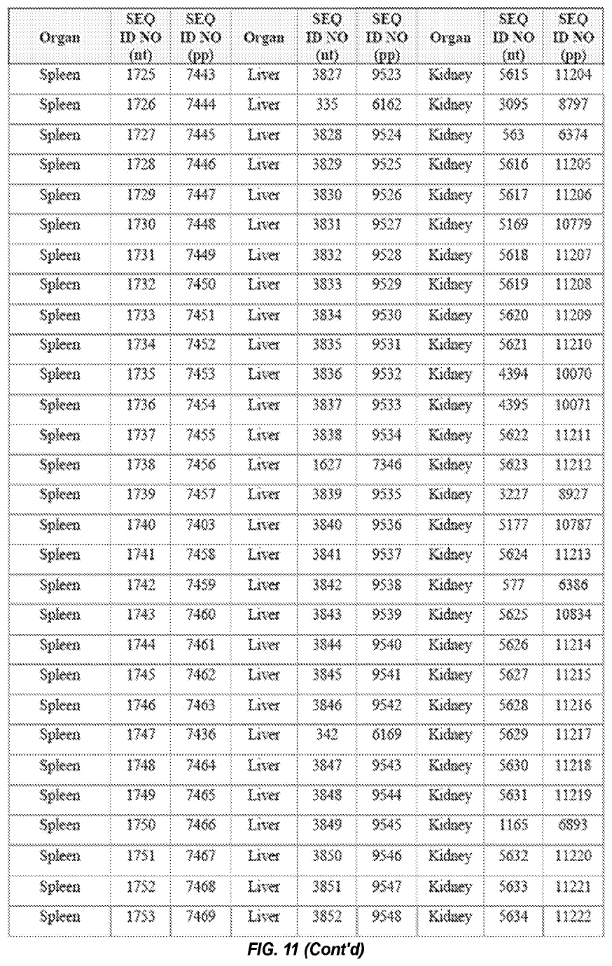

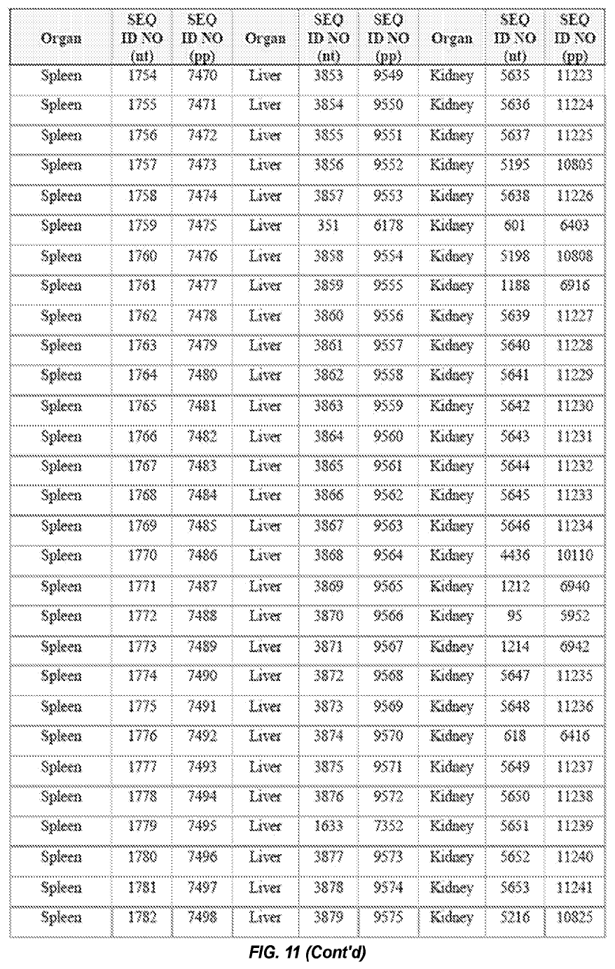

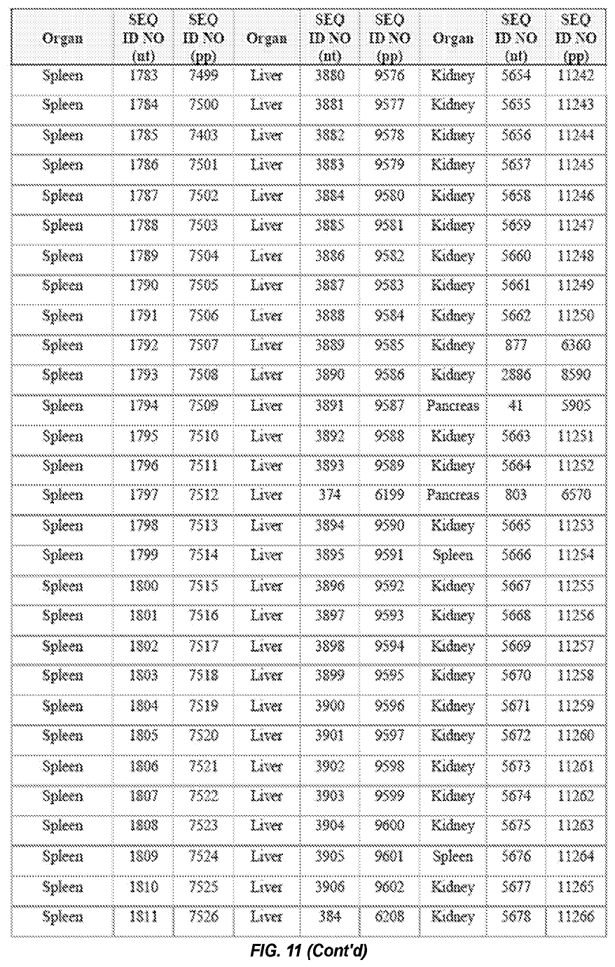

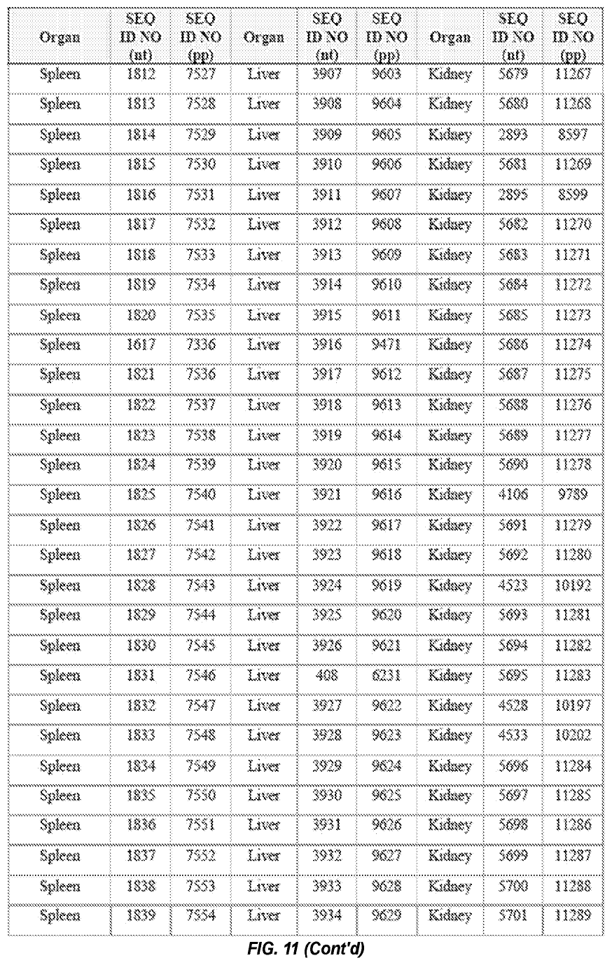

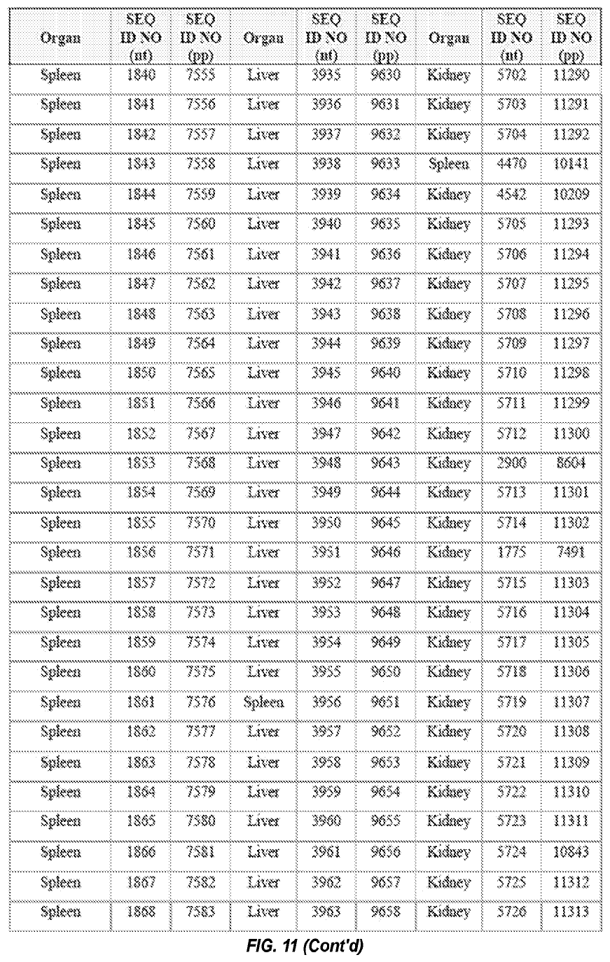

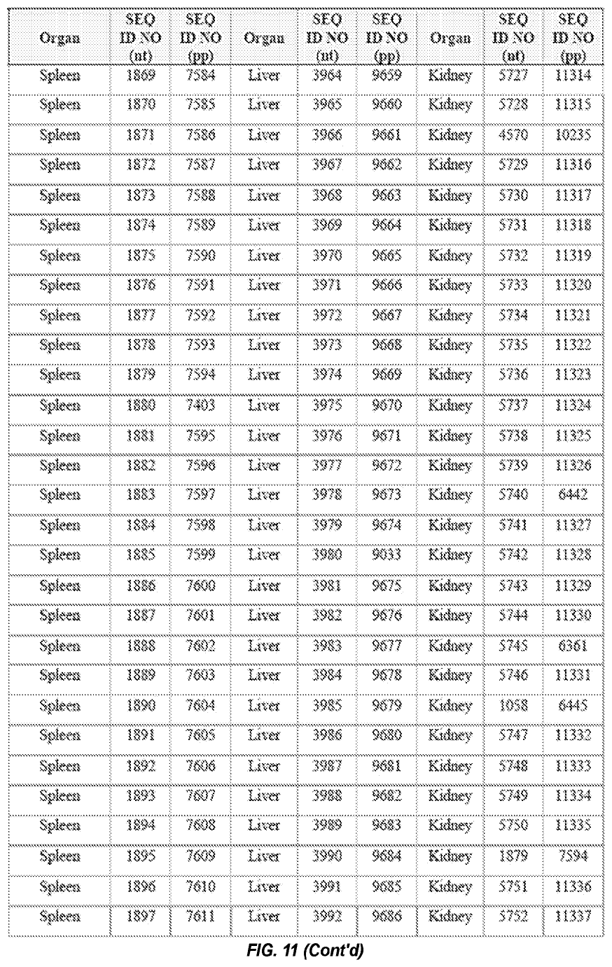

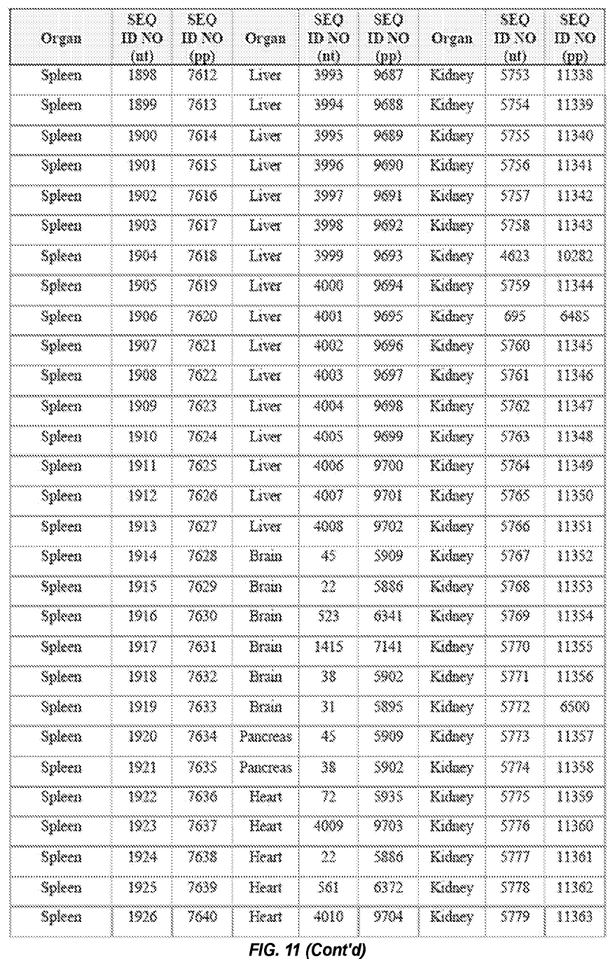

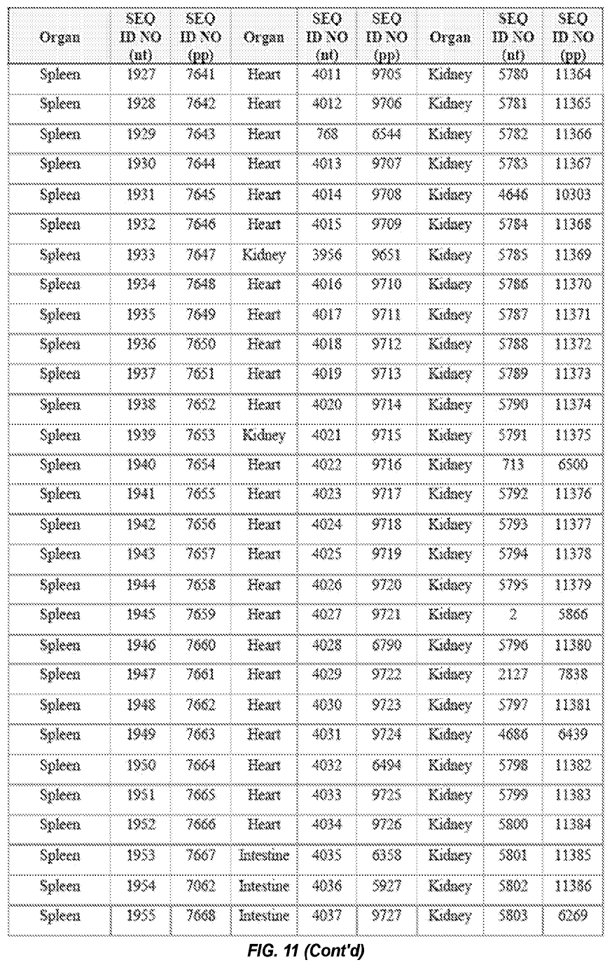

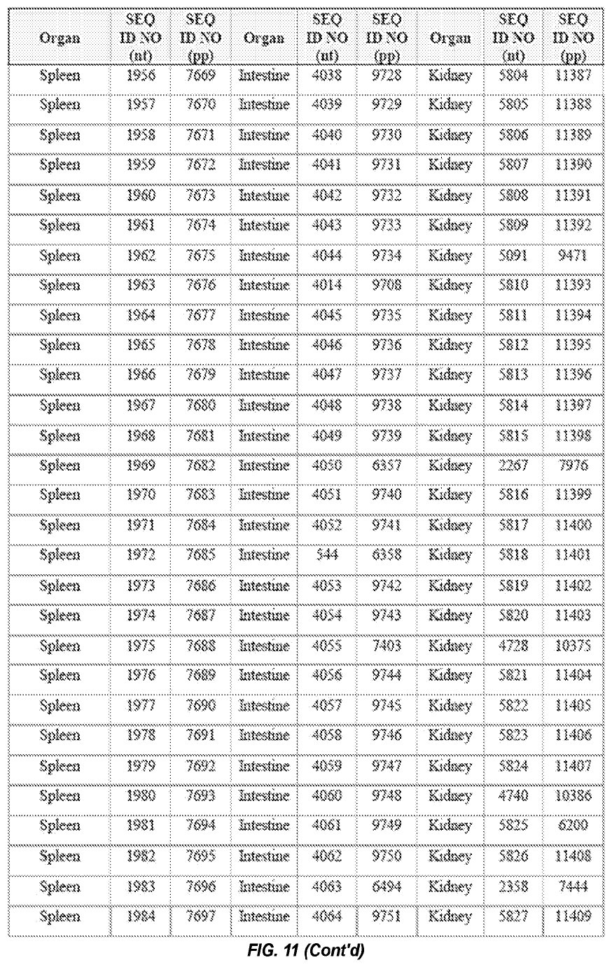

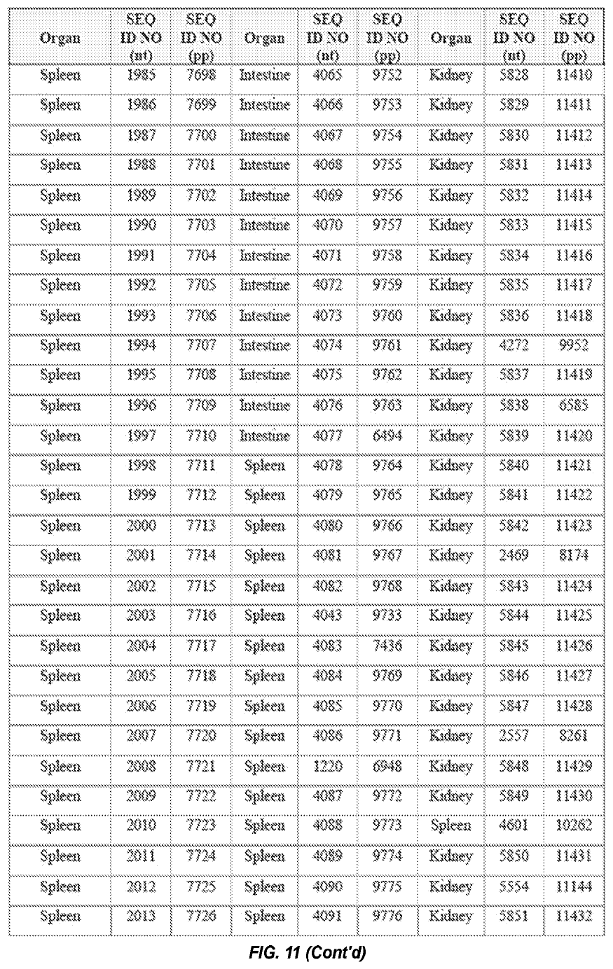

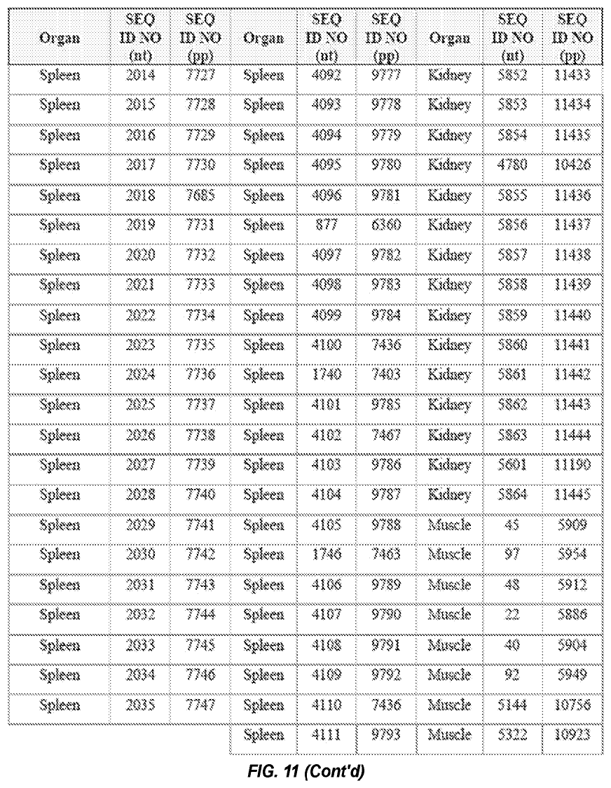

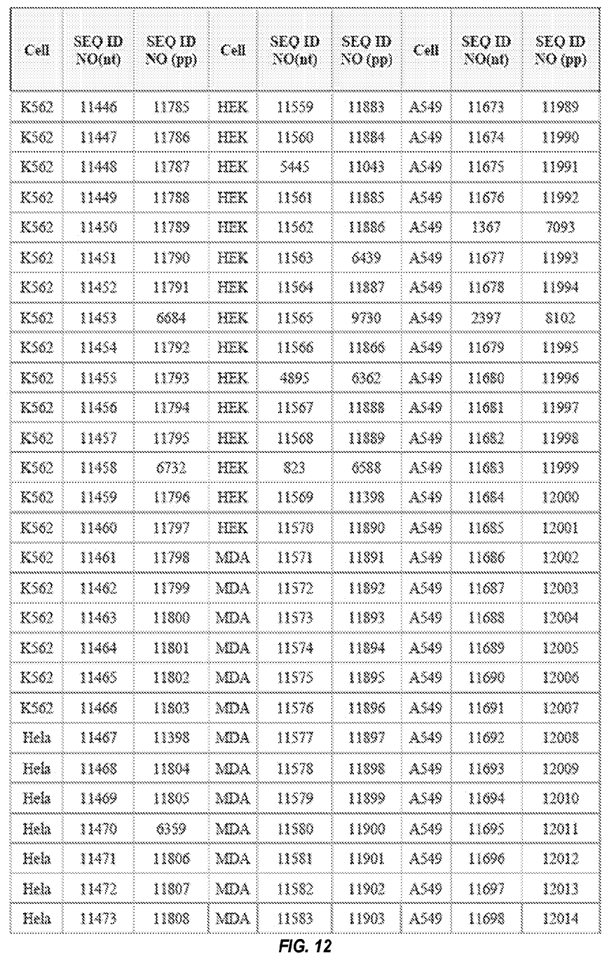

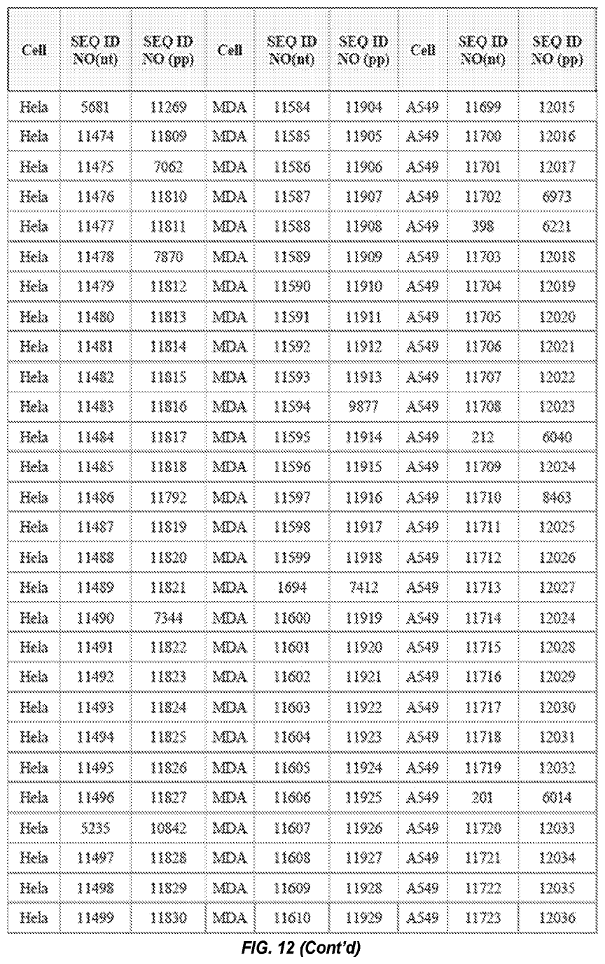

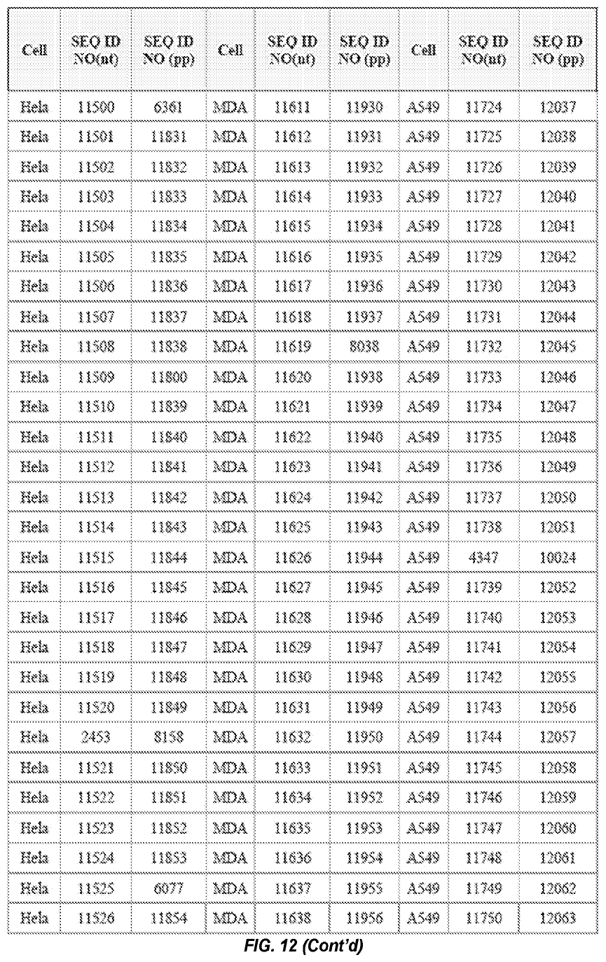

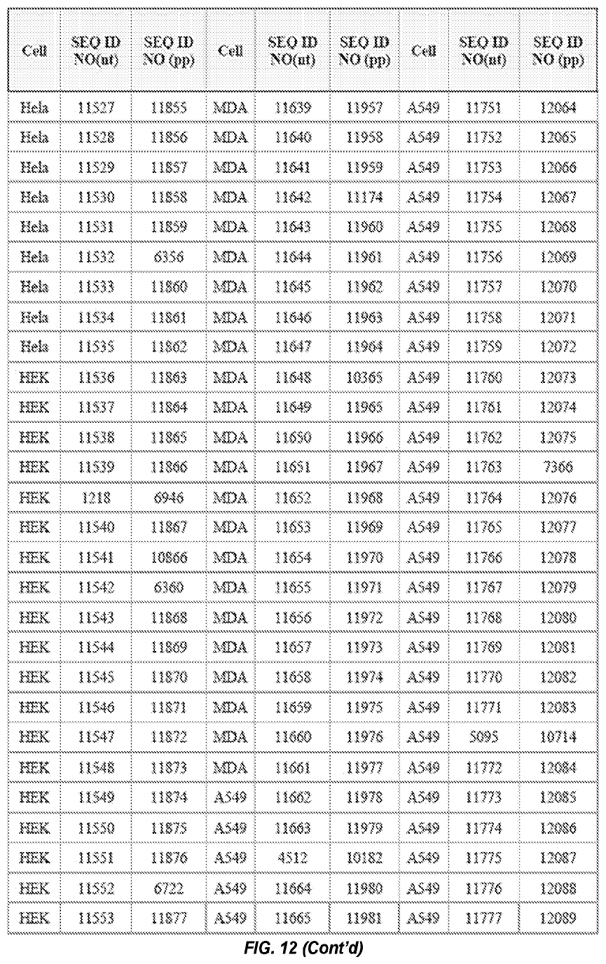

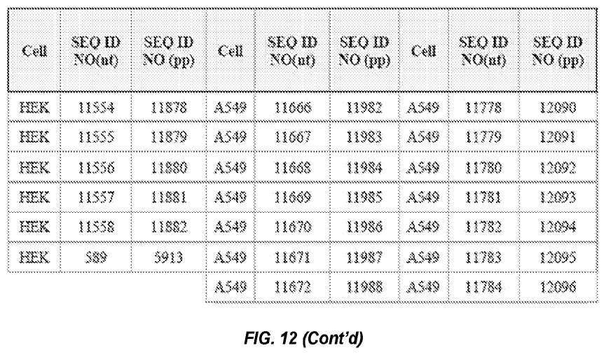

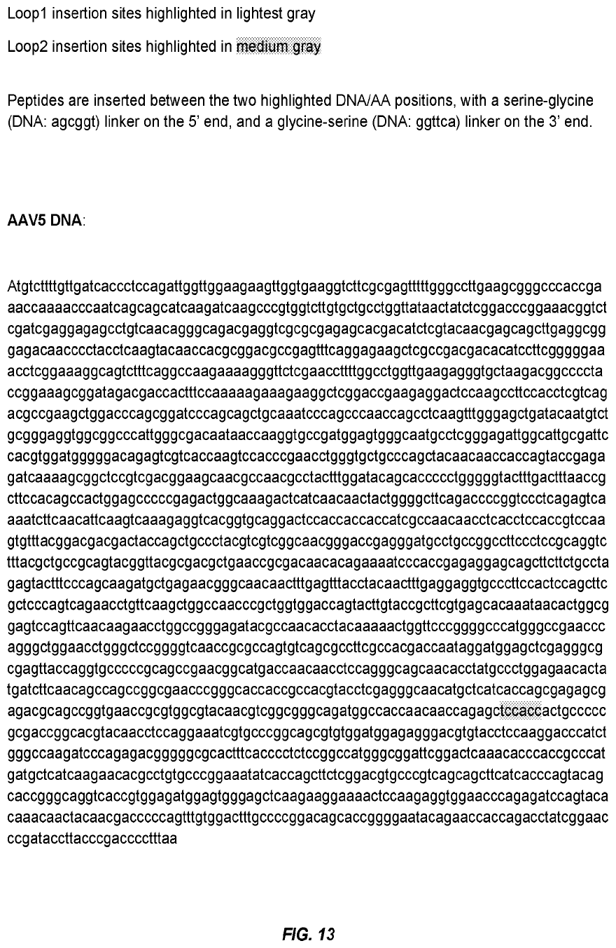

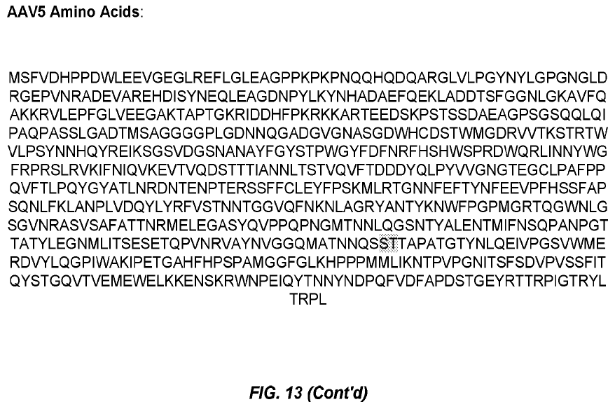

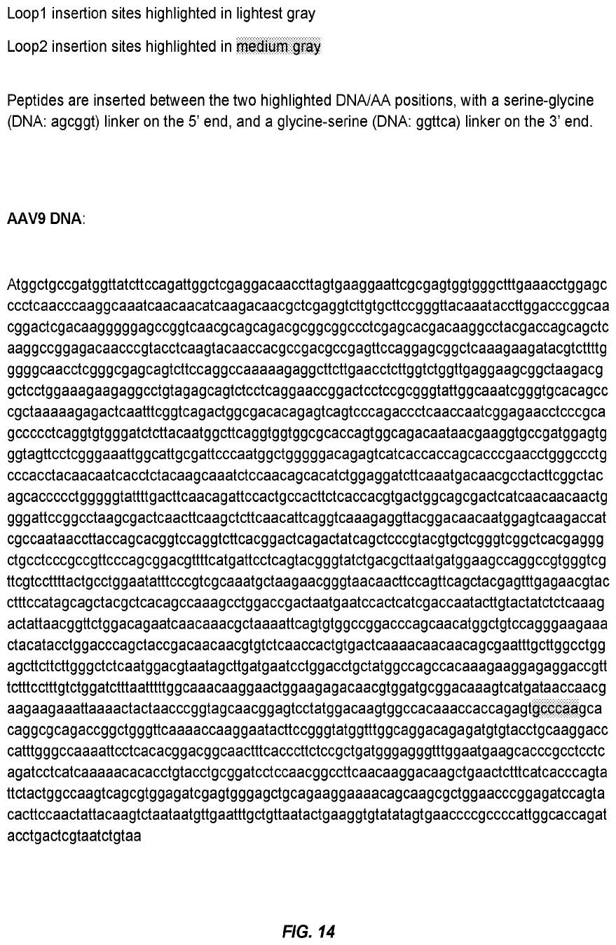

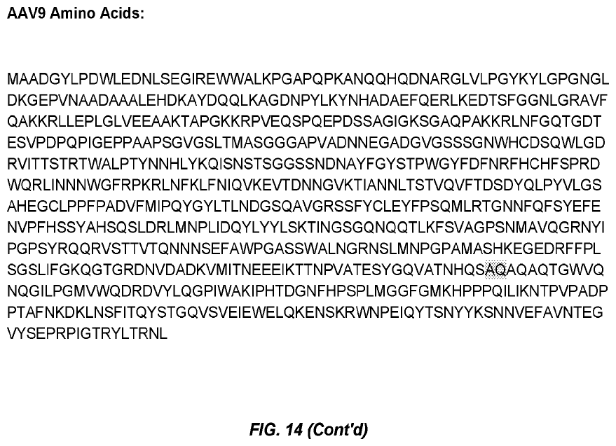

The design and implementation of displaying a large, tiled peptide library in the capsid of multiple viral (e.g., AAV) serotypes is shown in the studies presented herein. The disclosure further provides methodology for performing an in vivo screen to identify transducing viral variants across multiple organs, the analytical pipeline to characterize the top-performing hits, and finally, the identity and potential application of top performing viral variants is also discussed herein. Hits from a targeting peptide screen with AAV and Lentivirus and their coding sequences are provided in the sequence listing and their targeted tissue or cells are presented in FIG. 11 and FIG. 12.

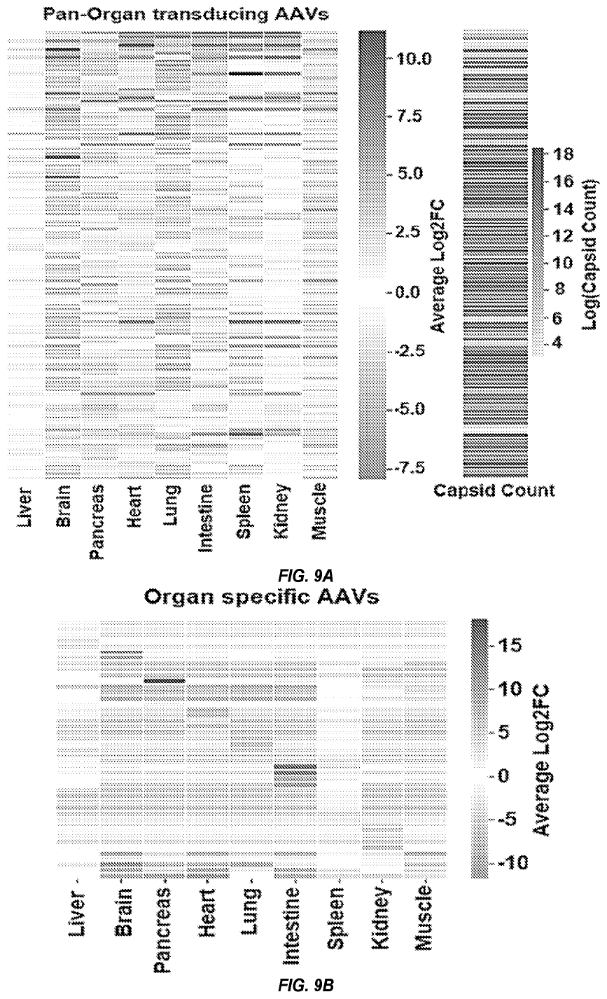

The heterologous targeting peptide-modified AAV variants identified here could be of broad use both clinically and experimentally. Based upon preliminary in vivo screening data, an expansive list of AAV variants with broad organ tropism were identified, these variants exhibited high log₂foldchange values in multiple organs in comparison to overall counts in the AAV capsid pool (see FIG. 9A). The ability of these AAV variants to transduce multiple organs at relatively low administered AAV doses allows for a lower overall titer of AAV that needs to be produced and delivered to achieve the same therapeutic benefit as current wild-type AAVs. This is especially important for reducing the overall costs of viral production, as well as limiting physiological exposure to the AAV vector in an effort to address the inherent immunogenicity of these gene delivery vehicles which has hindered prior efficacy of gene therapies in humans.

In addition to the broadly infective AAV variants discovered herein, the methods of the disclosure also identified several variants which enable organ-specific AAV transduction. These include AAV variants which specifically transduce the brain, lung, heart, skeletal muscle, pancreas, kidney, spleen, intestine, and liver (see FIG. 9B). Specific examples of the clinical utility of these organ-specific gene delivery vehicles are described in the following sections, however, any disease or condition in which a gene or genetic engineering protein should be expressed in a specific tissue or cell type could utilize the heterologous targeting peptide-modified AAV variants disclosed herein and achieve specific expression without the need for specialized promoters.

In the brain, a difficult organ to transduce due to the selectivity of the blood brain barrier, there are numerous diseases which would benefit from AAV that only target cell types in the central nervous system. Previous efforts to transduce the CNS have relied on difficult routes of administration such as intrathecal, intraparenchymal, and intracerebroventricular. The heterologous targeting peptide-modified AAV variants provided herein were shown to effectively transduce CNS cell types via systemic administration. For monogenic neurological disorders caused by specific gene mutations, these could be addressed with organ-specific AAV variants to address loss-of-function mutations (e.g. replacing survival motor neuron protein (SMN) for spinal muscular atrophy) or silencing harmful gain-of-function mutations (e.g. superoxide dismutase 1 (SOD1) for amyotrophic lateral sclerosis or huntingtin (HTT) for Huntington's disease. Other CNS diseases which currently have gene therapy based clinical trials ongoing, which would benefit from brain-specific AAV variants include Parkinson's disease, mucopolysaccaridosis type I, II and III, Batten disease, giant axonal neuropathy, and metachromatic leukodystrophy. Similarly, while it was not directly assessed in the initial screen, the AAV-engineering approach could greatly improve transgene delivery to the retina where mutations in >270 genes are implicated in hereditary retinal degeneration and could be addressed with an AAV-based gene therapy.

A similar paradigm exists in the lung where several diseases resulting from specific gene mutations have been addressed with AAVs administered through difficult means (i.e., intratracheal). Lung-specific AAV variants were identified with the methods presented herein, and therefore, the methods and compositions of the disclosure can be used to generate lung-specific AAV variants to treat various lung disease. Diseases which could be addressed include, but are not limited to, surfactant protein B deficiency, pulmonary vascular leakage, cystic fibrosis, and other lung obstructive diseases.

In other tissues, there is a vast array of clinical scenarios in which organ-specific AAV variants would be useful. Some examples include AAT deficiency and hepatitis in the liver, Duchenne and limb-girdle muscular dystrophy, sporadic inclusion body myositis, and dysferlin deficiency in the muscle, diabetes mellitus in the pancreas, and recovery from myocardial infarction and heart failure in the heart. Examples also include tissue specific cancer in which gene therapies could be packaged into the AAV variants to deliver anti-angiogenic factors, toxic genes, cytokines, tumor suppressor gene, antigenic vaccines, or antibodies specifically to cancer cells.

Additionally, the heterologous targeting peptide-modified AAV variants have been characterized on a tissue level in the studies presented herein, their tropism could be further investigated at the cellular level to enable cell-type specific targeting of transgene delivery. This could be of significant importance in an immune setting, for instance, where delivery of key transcription factors to regulatory T cells could provide a new treatment paradigm for autoimmune disorders or delivery of targeting receptors to cytotoxic T cells could transform the field of immuno-oncology. In the brain, using the platform to specifically target microglia could enable novel treatment approaches for diseases such as Alzheimer's and Parkinson's. Furthermore, while the modified AAV vectors presented thus far make use of a single peptide insertion site on the AAV capsid. It is postulated that dual-insertion of an identified peptide at both the “Loop1” and “Loop2” sites could further improve the specificity of transduction.

Due to the rational design approach of the methodology disclosed herein, the heterologous targeting peptide-modified AAV variants disclosed herein could be broadly utilized for basic research purposes. AAVs do not traditionally transduce cells in culture very effectively, which is especially true for primary cultures. The heterologous targeting peptide-modified AAV variants identified using the methods disclosed herein could enhance transduction and delivery of transgenes, greatly enabling basic research studies which were not previously feasible.

The heterologous targeting peptide-modified lentivirus variants identified here could be of broad use both clinically and experimentally. Tissue or cell type specific lentiviral particles have many potential applications. Lentiviral particles which can more efficiently transduce immune cells could have great utility for engineering chimeric antigen receptor (CAR) T cells for clinical applications. Additionally, tissue or cell type specific lentiviral particles could have great utility as basic science reagents. For example, being able to transduce a single cell type in a mixed population opens up unique screening avenues for perturbing cells of interest while sparing bystanders. More specifically, modern 3-dimensional tissue culture often makes use of multiple cell types to more accurately model tissue level physiology and architecture. A cell-type specific lentivirus could thus be applied to 3-dimensional tissue models to genetically modify only a subset of the cells which make up the tissue. Beyond the direct applications of engineered lentiviruses, the peptides discovered by this screening methods disclosed herein could also be applied to alternative gene and drug delivery modalities. For example, peptides identified using the methods disclosed herein could be used as targeting ligands for lipid nanoparticles, fused to therapeutic proteins to enable targeted delivery, or even integrated into other viral particles with different wild-type tropism. Targeting of lipid nano-particles is of special interest, due to the similarities between a lipid nanoparticle and the lipid wrapped structure of a lentivirus. Re-targeted lipid nano-particles have immense therapeutic potential, in fields such as vaccine delivery, gene therapy, or targeted imaging.

As indicated above, any number of virus and viral particles may be coated using the methods disclosed herein, including enveloped viruses, such as retroviruses. In regards to enveloped viruses, the viruses may be first treated to remove the viral envelope, and then coated with membrane fragments using the methods disclosed herein. Examples of method to remove viral envelopes include use of detergents, like triton-X 100, and 3-[(3-cholamidopropyl)dimethylammonio]-1-propanesulfonate (CHAPS); use of alcohols, like ethanol and 2-propanol; heat treatment; and drying.

The disclosure provides a method for coating a virus or viral particle with membrane fragments comprising one or more of the following steps: lysing cells in a hypotonic solution, which optionally may be combined with Dounce homogenization or sonication, in order to fractionate the cell membrane; removing cells and cell debris by differential centrifugation, leaving a membrane enriched fraction; pelleting and extruding the membrane fragments through polycarbonate membrane(s) to generate purified membrane fragments; coating virus or viral particles by coextruding the virus or viral particles with the purified membrane fragments through polycarbonate membrane(s).

The cells that are used to generate the membrane fragments disclosed herein, are referred to herein as donor cells. The donor cells are generally eukaryotic in origin and are typically mammalian cells. The donor cells can originate from any number of different types of organisms, including from humans, mice, rats, rabbits, sheep, goats, non-human primates, dogs, etc. Moreover, the methods disclosed herein are not limited to a particular donor cell type, and the membrane fragments can be generated from a variety of different cells types, including, but not limited to, stem cells, progenitor cells, primary cells, somatic cells, germline cells, tumor cells, cell lines, etc. Thus, the methods of the disclosure have general applicability and can be used with different types of cells, from different organisms. Further, the donor cells can be selected so as to generate a coated viruses or viral particle for a specific purpose, for example delivering to a specific cell or tissue type, or improving the efficiency of the therapy.

The donor cells may be modified such that the membrane fragments generated therefrom further comprise a targeting ligand. Such a targeting ligand may be used to direct the coated viruses or viral particles of the disclosure to specific cells with which they will ultimately fuse. Such a targeting ligand can be produced, for example, by engineering the donor cells to express a cell surface anchoring motif comprising a targeting ligand (e.g., a neuron-specific RVG peptide), or by affixing peptides to membrane proteins via maleimide based linkers. Examples of cell surface anchoring/display motifs include, but are not limited to, outer membrane proteins, lipoproteins, glycosylphosphatidylinositol (GPI) anchoring motifs, and autotranspoters. The targeting ligand can be a member of a specific binding pair. the other of which is found on the target cells (Alvarez-Erviti et al., Nature Biotechnology 29:341-345 (2011)). In one embodiment, the targeting ligand is an antibody or antigen binding fragment thereof (e.g., a single chain antibody (scFV)) that specifically binds a marker present on a cellular target. In another embodiment, the targeting ligand is a short homing peptide. Examples of sequences for short homing peptides include, but are not limited to, ATWLPPR, NGR, CRTLTVRKC, CRKRLDRNC, SPSYVYHQF, SVYDFFVWL, aKXVAAWTLKAAaZC, SFERFEIFPKEC, CRGDKCPDC, KLWVLPKGGGCAm, CSKSSDYQC, hTrail (114-281), TFFYGGSRGKRNNFKTEEY, CGNKRTR, CHVLWSTRC, CDLRSAAVC, cRGFfK, cdG-HoCit-GPQc-Ebes-K-alkyne, HLNILSTLWKYR, cyclic RGD, c(RGDyK), cRGD, c(RGDyK), and GRGDS.

One of the primary advantages of the coated viruses or viral particles disclosed herein is avoiding immunodetection in a subject. More specifically, the coated virus or viral particles disclosed herein will not be recognized by the human immune system as a pathogen because the outer cell membrane has a human source, containing proteins that prevent neutralization by immune cells.

Immunity can be broadly defined as all the processes that enable an organism to defend itself against antigens perceived as causing a rupture of homeostatic welfare. Since recombinant AAV vectors do not contain any viral gene, the only sources of foreign antigens brought in during gene transfer are derived from the viral capsid and the transgene product. The nucleic acid contained in the virion may also concur to activate immunity via engagement of Toll-like receptors. The prevalence of total anti-AAV antibodies is close to 70% of the population for AAV1 and AAV2, 45% for AAV6 and AAV9), and 38% for AAV8. Importantly, titers of anti-AAV immunoglobulin G (IgG) antibodies correlate significantly, though not completely, with titers of anti-AAV neutralizing antibodies. Anti-capsid cellular responses are less preponderant than humoral responses. Correlation studies between anti-AAV humoral and cellular responses suggest that there is no link between both parameters, at least for the AAV1 and AAV2 serotypes.

While rAAV vectors do not encode viral proteins, the viral particles have an identical composition to WT AAV. Therefore, high doses of rAAV vectors can potentially activate recall responses generated against WT AAV capsid following cross-presentation of capsid antigens on target cells. The easiest way to bypass the impact of pre-existing immune responses to AAV would be simply to exclude from clinical trials the subjects exhibiting high amounts of anti-AAV antibodies/neutralizing factors or capsid-reactive T cells. Considering that AAV-seropositive individuals represent up to 70% of the population, exclusion is difficult. Similarly, pre-screening patients to exclude those with pre-existing anti-AAV cellular immunity is not a sound approach, as the frequency of pre-existing circulating AAV-specific T cells in PBMCs is too low to permit their systematic detection through ELISpot or flow cytometry assays. Furthermore, positive anti-capsid cellular responses in clinical trials are not systematically translated into deleterious clinical consequences, and there is currently no means of predicting which parameters will trigger the onset of harmful responses. Importantly, though anti-AAV immune responses can result in loss of transgene expression, they do not inflict other harmful sequelae to the patient and seem to be so far more an “efficiency” than a “safety” issue. The best trade-off one can currently imagine is to engineer rAAV vectors with better transduction efficiency, carrying optimized therapeutic transgenes and with reduced immunogenic profiles. Such vectors would provide a higher therapeutic index, as they would permit therapeutic efficiency at doses sufficient to bypass pre-existing humoral immunity, but not high enough to trigger deleterious cellular immunity.

Accordingly, the methods of the disclosure can greatly improve the efficacy and safety of current gene therapies, many of which use adeno-associated viruses due to their low pathogenicity and high degree of infectivity. Because AAVs (as well as the vast majority of gene therapy products) do not mediate lifelong transgene expression, there is considerable need for a method to bypass the immune system and enable repeat dosing. Encapsulating the AAV (or other gene therapy vector) in non-immunogenic human cell membranes allows for multiple doses of immunogenic gene therapies to be safely administered to patients. As such, the coated viruses or viral particles provide for a higher therapeutic index than non-coated viruses, and would permit therapeutic efficiency at doses sufficient to bypass pre-existing humoral immunity (e.g., humoral immunity to AAV), but not high enough to trigger deleterious cellular immunity. Further, the coated viruses and viral particles made by the methods disclosed herein can be further modified to provide for targeted delivery by the selection of the specific types of donor cells and/or use of targeting ligands. Additionally, peptide sequences can be added to the viral capsid to improve transduction and inhibit the pathway that presents viral antigens to immune cells (i.e., Major Histocompatibility complex). Such peptide sequences can be identified using the methods described in WO 2018/170015, the disclosure of which is incorporated herein in-full, which describes identifying one or more regions of an AAV capsid protein with affinity for a major histocompatibility complex (MHC), and modifying the one or more regions of the AAV capsid protein with affinity for the MHC through one or more amino acid substitutions, such that the modified region has no affinity for the MHC. Additionally, directed evolution can be used to rapidly engineer viruses or viral particles with desired gene delivery properties, including neutralizing antibody-evasion properties, as well as enhanced gene delivery, gene targeting, and enhanced capacity to infect. Such directed evolution strategies for creating virus variants with improved properties are described in the following references, which disclosure of which are incorporated herein in-full, Asuri et al., Molecular Therapy 20(2):329-338 (2012)); Maheshri et al., Nat Biotechnol 24:198-294 (2006); Koerber et al., Mol Ther 16:1703-1709 (2008); Koerber et al., Mol Ther 17:2088-2095 (2009); Klimczak et al., PLoS One 4:e7467 (2009); Exoffon et al., Proc Natl Acad Sci USA 106:3865-3870 (2009); Li et al., Mol. Ther 17:2067-2077 (2009); and Grimm et al., J Virol 82:5887-5911 (2008)). By preventing the presentation of antigens and/or by improving the evasion properties of the viruses to antibody neutralization, the immune stealth qualities of the coated viruses or viral particles of the disclosure can be improved without having to administer immunosuppressants, which may have deleterious effects.

The coated viruses and viral particles made by the methods of the disclosure can be used to treat diseases which can be ameliorated by the delivery or the actions of viral gene therapy, or other therapies (e.g., antisense oligonucleotide, small molecule therapeutics, genome engineering, etc.) on the target cells. In one embodiment, the disease involves or is caused by a genetic deficiency in the target cells. The molecule for which they are deficient (or encoding the molecule for which they are deficient) can be delivered to the appropriate cells via the coated viruses or viral particles disclosed herein.

The disclosure further provides methods of delivering viral gene therapy, or other therapy, to a subject comprising, administering an effective amount of a coated virus or viral particle preparation produced by a method disclosed herein to the subject. The administering can be local or systemic. For example, the coated virus or viral particle preparation disclosed herein may be locally administered to a subject by injection, such as by injection into an organ or a tumor.

The disclosure also provides methods of delivering a viral gene therapy, or other therapy to a cell, comprising: contacting the cell with an effective amount of a coated virus or viral particle preparation produced by a method disclosed herein that comprises a gene therapy. In one embodiment, the cell is contacted in vivo. In a further embodiment, the cell is contacted within an organ or tumor. In yet a further embodiment, the coated virus or viral particle preparation is produced ex vivo from donor cells of a subject. In an alternate embodiment, the cell is contacted in vitro.

The disclosure further provides methods of delivering a CRISPR-Cas genome engineering system, or other type of genome engineering system to a cell, comprising: contacting the cell with an effective amount of a coated virus or viral particle preparation produced by a method disclosed herein which comprises a CRISPR-Cas genome engineering system. In one embodiment, the cell is contacted in vivo. In a further embodiment, the cell is contacted within an organ or tumor. In yet a further embodiment, the coated virus or viral particle preparation is produced ex vivo from donor cells of a subject. In an alternate embodiment, the cell is contacted in vitro.

The disclosure further provides for pharmaceutical compositions, formulations and preparations comprising a coated virus or viral particle described herein for specified modes of administration. In one embodiment, a coated virus or viral particle described herein is an active ingredient in a composition comprising a pharmaceutically acceptable carrier. Such a composition is referred to herein as a pharmaceutical composition. A “pharmaceutically acceptable carrier” means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier should be “acceptable” in the sense of being compatible with the other ingredients of the composition and is compatible with administration to a subject, for example a human. Such compositions can be specifically formulated for administration via one or more of a number of routes, such as the routes of administration described herein. Supplementary active ingredients also can be incorporated into the compositions. When an agent, formulation or pharmaceutical composition described herein, is administered to a subject, preferably, a therapeutically effective amount is administered. As used herein, the term “therapeutically effective amount” refers to an amount that results in an improvement or remediation of the condition.

Administration of the pharmaceutical composition to a subject is by means which the coated viruses or virial particle contained therein will contact the target cell. The specific route will depend upon certain variables such as the target cell and can be determined by the skilled practitioner. Suitable methods of administering a coated virus or viral particle described herein to a patient include any route of in vivo administration that is suitable for delivering a coated virus or viral particle to a patient. The preferred routes of administration will be apparent to those of skill in the art, depending on the preparation's type of viral gene therapy being used, the target cell population, and the disease or condition experienced by the subject. Preferred methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. In an embodiment where the target cells are in or near a tumor, a preferred route of administration is by direct injection into the tumor or tissue surrounding the tumor. For example, when the tumor is a breast tumor, the preferred methods of administration include impregnation of a catheter, and direct injection into the tumor.

Intravenous, intraperitoneal, and intramuscular administrations can be performed using methods standard in the art. Aerosol (inhalation) delivery can also be performed using methods standard in the art (see, for example, Stribling et al., Proc. Natl. Acad. Sci. USA 189: 11277-11281, 1992, which is incorporated herein by reference in its entirety). Oral delivery can be performed by complexing an extracellular vesicle preparation of the present invention to a carrier capable of withstanding degradation by digestive enzymes in the gut of an animal. Examples of such carriers, include plastic capsules or tablets, such as those known in the art.

One method of local administration is by direct injection. Direct injection techniques are particularly useful for administering the coated virus or viral particle to a cell or tissue that is accessible by surgery, and particularly, on or near the surface of the body. Administration of a composition locally within the area of a target cell refers to injecting the composition centimeters and preferably, millimeters from the target cell or tissue.

The appropriate dosage and treatment regimen for the methods of treatment described herein will vary with respect to the particular disease being treated, the coated virus or viral particle being delivered, and the specific condition of the subject. The skilled practitioner is to determine the amounts and frequency of administration on a case-by-case basis. In one embodiment, the administration is over a period of time until the desired effect (e.g., reduction in symptoms is achieved). In a certain embodiment, administration is 1, 2, 3, 4, 5, 6, or 7 times per week. In a particular embodiment, administration is over a period of 1, 2, 3, 4, 5, 6, 7, 8, 9, or 10 weeks. In another embodiment, administration is over a period of 2, 3, 4, 5, 6 or more months. In yet another embodiment, treatment is resumed following a period of remission.

For use in the applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more agents for coating viruses or viral particles described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The disclosure further provides that the methods and compositions described herein can be further defined by the following aspects (aspects 1 to 87):
1. A viral vector having a capsid protein comprising a heterologous targeting peptide in a range of 10-30 amino acids in length.
2. The viral vector of aspect 1, wherein the heterologous targeting peptide is about 15-25 amino acids in length.
3. The viral vector of aspect 1 or 2, wherein the heterologous targeting peptide is about 20 amino acids in length.
4. The viral vector of any one of aspects 1-3, wherein the viral vector is an adeno-associated virus (AAV).
5. The viral vector of any one of aspect 1-3, wherein the viral vector is a lentiviral vector.
6. The viral vector of any one of aspects 1 to 5, wherein the capsid protein is a VP1 capsid protein.
7. The viral vector of any one of aspects 1 to 5, wherein the capsid protein is a VP2 capsid protein.
8. The viral vector of any one of aspects 1 to 5, wherein the capsid protein is a VP3 capsid protein.
9. The viral vector of any one of any one of aspects 1-4 and 6-8, wherein the heterologous targeting peptide is inserted into an AAV capsid protein at loop 1 and/or loop 2.
10. The viral vector of any one of aspects 1-3 or 9, wherein the viral vector is an AAV5.
11. The viral vector of any one of aspects 1-4 and 6-9, wherein the viral vector is an AAV9.
12. The viral vector of any one of aspects 1-11, wherein the heterologous targeting peptide is flanked by a linker peptide at the N-terminal and C-terminal ends of the heterologous targeting peptide.
13. The viral vector of any one of aspects 1-11, wherein the heterologous targeting peptide is not flanked by a linker peptide at the N-terminal and C-terminal ends of the heterologous targeting peptide.
14. The viral vector of any one of aspects 1-13, wherein the heterologous targeting peptide targets the viral vector to hepatocytes or liver tissue.
15. The viral vector of any one of aspects 1-13, wherein the heterologous targeting peptide targets the viral vector to neuronal cells or brain tissue.
16. The viral vector of any one of aspects 1-13, wherein the heterologous targeting peptide targets the viral vector to pancreatic cells or pancreas tissue.
17. The viral vector of any one of aspects 1-13, wherein the heterologous targeting peptide targets the viral vector to cardiac cells or heart tissue.
18. The viral vector of any one of aspects 1-13, wherein the heterologous targeting peptide targets the viral vector to lung tissue.
19. The viral vector of any one of aspects 1-13, wherein the heterologous targeting peptide targets the viral vector to intestinal tissue.
20. The viral vector of any one of aspects 1-13, wherein the heterologous targeting peptide targets the viral vector to spleen tissue.
21. The viral vector of any one of aspects 1-13, wherein the heterologous targeting peptide targets the viral vector to renal cells or kidney tissue.
22. The viral vector of any one of aspects 1-13, wherein the heterologous targeting peptide targets the viral vector to muscle cells or tissue.
23. An adeno-associated virus (AAV) capsid protein comprising a heterologous targeting peptide cloned into loop 1 and/or loop 2 of the capsid protein, wherein the heterologous targeting peptide is about 10-30 amino acids in length.
24. The AAV capsid protein of aspect 23, wherein the capsid protein is a VP1 capsid protein.
25. The AAV capsid protein of aspect 23, wherein the capsid protein is a VP2 capsid protein.
26. The AAV capsid protein of aspect 23, wherein the capsid protein is a VP3 capsid protein.
27. The AAV capsid protein of aspect 23 to 26, wherein the heterologous targeting peptide is about 15-25 amino acids in length.
28. The AAV capsid protein of any one of aspects 23 to 27, wherein the heterologous targeting peptide is about 20 amino acids in length.
29. The AAV capsid protein of any one of aspects 23 to 28, wherein the heterologous targeting peptide is flanked by a linker peptide at the N-terminal and C-terminal ends of the heterologous targeting peptide.
30. The AAV capsid protein of any one of aspects 23 to 29, wherein the heterologous targeting peptide targets hepatocytes or liver tissue.
31. The AAV capsid protein of any one of aspects 23 to 29, wherein the heterologous targeting peptide targets neuronal cells or brain tissue.
32. The AAV capsid protein of any one of aspects 23 to 29, wherein the heterologous targeting peptide targets pancreatic cells or pancreas tissue.
33. The AAV capsid protein of any one of aspects 23 to 29, wherein the heterologous targeting peptide targets cardiac cells or heart tissue.
34. The AAV capsid protein of any one of aspects 23 to 29, wherein the heterologous targeting peptide targets lung tissue.
35. The AAV capsid protein of any one of aspects 23 to 29, wherein the heterologous targeting peptide targets intestinal tissue.
36. The AAV capsid protein of any one of aspects 23 to 29, wherein the heterologous targeting peptide targets spleen tissue.
37. The AAV capsid protein of any one of aspects 23 to 29, wherein the heterologous targeting peptide targets renal cells or kidney tissue.
38. The AAV capsid protein of any one of aspects 23 to 29, wherein the heterologous targeting peptide targets muscle cells or tissue.
39. A recombinant AAV (rAAV) comprising a capsid protein of any one of aspects 23-38.
40. A recombinant AAV (rAAV) comprising a capsid protein having a targeting peptide in loop 1 and/or loop 2 wherein the targeting peptide is independently selected from SEQ ID Nos:5865 to 11445.
41. The recombinant AAV of aspect 40, wherein the recombinant AAV further comprises a heterologous polynucleotide for gene delivery.
42. The recombinant AAV of aspect 41, wherein the heterologous polynucleotide is a therapeutic gene.
43. A composition comprising the recombinant rAAV of any one of aspects 40-42.
44. The composition of aspect 43 further comprising a pharmaceutically acceptable carrier.
45. A method for delivering a transgene to a subject comprising:
   administering a recombinant AAV (rAAV) to a subject, wherein the rAAV comprises:
      (i) a capsid protein of any one of aspects 23-38, and
      (ii) at least one transgene, and wherein the rAAV infects cells of a target tissue of the subject.
46. The method of aspect 45, wherein the at least one transgene encodes a protein.
47. The method of aspect 46, wherein the protein is an immunoglobulin heavy chain or light chain or fragment thereof.
48. The method of aspect 45, wherein the at least one transgene encodes a small interfering nucleic acid.
49. The method of aspect 48, wherein the small interfering nucleic acid is a miRNA.
50. The method of aspect 48, wherein the small interfering nucleic acid is a miRNA sponge or TuD RNA that inhibits the activity of at least one miRNA in the subject or animal.
51. The method of aspect 49, wherein the miRNA is expressed in a cell of the target tissue.
52. The method of aspect 45, wherein the target tissue is skeletal muscle, heart, liver, pancreas, brain or lung.
53. The method of aspect 45, wherein the transgene expresses a transcript that comprises at least one binding site for a miRNA, wherein the miRNA inhibits activity of the transgene, in a tissue other than the target tissue, by hybridizing to the binding site.
54. The method of aspect 45, wherein the at least one transgene encodes a gene product that mediates genome editing.
55. The method of aspect 45, wherein the transgene comprises a tissue specific promoter or inducible promoter.
56. The method of aspect 55, wherein the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter.
57. The method of any one of aspects 45 to 56, wherein the rAAV is administered intravenously, intravascularly, transdermally, intraocularly, intrathecally, orally, intramuscularly, subcutaneously, intranasally, or by inhalation.
58. The method of any one of aspects 45 to 57, wherein the subject is selected from a mouse, a rat, a rabbit, a dog, a cat, a sheep, a pig, and a non-human primate.
59. The method of any one of aspects 45 to 57, wherein the subject is a human.
60. An isolated nucleic acid encoding an AAV capsid protein containing an amino acid sequence selected from the group consisting of SEQ ID No:5865 to 11444 and 11445.
61. A composition comprising the isolated AAV capsid protein of any one of aspects 23 to 38.
62. The composition of aspect 61 further comprising a pharmaceutically acceptable carrier.
63. A kit for producing a rAAV, the kit comprising: a container housing an isolated nucleic acid of aspect 60.
64. The kit of aspect 63 further comprising instructions for producing the rAAV.
65. The kit of aspect 63 or aspect 64, further comprising at least one container housing a recombinant AAV vector, wherein the recombinant AAV vector comprises a transgene.
66. A method for coating a virus or viral particle with membrane fragments comprising:
   lysing donor cells in a hypotonic solution, which optionally may be combined with Dounce homogenization or sonication, in order to fractionate the cell membrane;
   removing cells and cell debris by one or more rounds of centrifugation, leaving a membrane enriched fraction;
   extruding the membrane enriched faction through polycarbonate membrane(s) to generate purified membrane fragments; and
   coating virus or viral particles by coextruding the virus or viral particles with the purified membrane fragments through polycarbonate membrane(s).
67. The method of aspect 66, wherein the viruses or viral particles are non-enveloped viruses or viral particles.
68. The method of aspect 66, wherein the viruses or viral particles are enveloped viruses or viral particles which have had their viral envelope removed.
69. The method of any one of aspects 66-68, wherein the viruses or viral particles are selected from retroviruses, adenovirus, adeno-associated virus, hybrid adenoviruses, alphavirus, herpes simplex virus, poxvirus, Epstein-Barr virus and lentivirus.
70. The method of aspect 69, wherein the viruses or viral particles are adeno-associated viruses (AAV).
71. The method of any one of aspects 66-70, wherein the viruses or viral particles have been modified by directed evolution to have increased neutralizing antibody-evasion properties, as well as enhanced gene delivery, gene targeting, and/or enhanced capacity to infect.
72. The method of any one of aspects 66-71, wherein the viruses or viral particles have been modified by one or more amino acid substitutions in one or more regions of a viral capsid protein so as to reduce the affinity of the viral capsid protein for the major histocompatibility complex.
73. The method of any one of aspects 66-71, wherein the donor cells are mammalian cells.
74. The method of aspect 73, wherein the donor cells are human cells.
75. The method of aspect 74, where the donor cells are human stem cells, human progenitor cells, human primary cells, human somatic cells, human germline cells, or human tumor cells.
76. The method of any one of aspects 66-75, wherein the membranes of the donor cells have been modified to express or present a targeting ligand.
77. The method of aspect 76, wherein the targeting ligand is used to improve entry of the coated viruses or viral particles into target cells, inhibit components of the immune response to the coated viruses or viral particles, or to target the coated viruses or viral particles to certain cell types or organs.
78. The method of aspect 76 or aspect 77, wherein the targeting ligand is a peptide, antibody or antibody fragment.
79. The method of aspect 78, wherein the targeting ligand comprises a peptide of any one of SEQ ID Nos:5865 to 11445.
80. Coated viruses or viral particles made by the method of any one of aspects 66-79.
81. The coated viruses or viral particles of aspect 80, wherein the coated viruses or viral particles have been modified to comprise a targeting ligand.
82. The coated viruses or viral particles of aspect 81, wherein the coated viruses or viral particles are used to deliver transgene(s) into target cells.
83. The coated viruses or viral particles of aspect 81 or aspect 82, wherein the coated viruses or viral particles are used to genome engineer target cells.
84. A pharmaceutical composition comprising the coated viruses or viral particles of any one of aspect 80 to 83 and a pharmaceutically acceptable carrier, diluent, binder and/or filler.
85. A method of treating a subject suffering from a disease or disorder in need of treatment thereof, comprising administering the coated viruses or viral particles of any one of aspects 80 to 83, or the pharmaceutical composition of aspect 84.
86. An engineered viral particle comprising an artificially prepared lipid envelope.
87. A method of preparing an engineered retroviral particle, the method comprising treating a retroviral particle with a detergent to remove a lipid envelop to obtain naked retroviral particles, isolating the naked retroviral particles and co-extruding a lipid envelop with the naked retroviral particles to obtain an engineered retroviral particle.

The following examples are intended to illustrate but not limit the disclosure. While they are typical of those that might be used, other procedures known to those skilled in the art may alternatively be used.

### EXAMPLES

Protocol for producing membrane bound AAVs: Adherent cells are grown to at least 70% confluency. The cells are detached by using 0.05% trypsin, pelleted by centrifuging at 500×g, and washed with PBS (×3). The cells are taken up in hypotonic lysis buffer: 0.2 mM EDTA in ddH₂O+protease inhibitor (1 mini tablet for a 10 mL solution). The suspension is then adjusted with 1× PBS and DNase is added. The lysed cells are then centrifuged at 18,000×g for 7 mins, the supernatant is removed, and the pelleted material is re-suspended in FBS (7×). After washing the pelleted material in 10 mM Tris-HCl+0.2 mM EDTA, the pelleted material is resuspended in ddH₂O and sonicated briefly (a few seconds). A series of extrusions are then performed using 400 nm, 200 nm, 100nm, and then 50 nm polycarbonate membranes to purify the membrane fragments. The resulting membrane fragments were then coated onto AAVs by co-extruding the membrane fragments and AAVs using 200 nm, 100 nm, and then 50 nm polycarbonate membranes. The coated AAVs are then centrifuged at 18,000×g for 10 mins in order to isolate membrane coated AAVs from empty membranes.

Alternate Protocol for Coating AAVs: The method used to lyse cells and create membrane fragments is based upon the methods described in Fang et al. (Nano Letters 14(4):2181-2188 (2014)). Briefly, the cells are placed in a hypotonic solution (consisting of 20 mM Tris-HCl pH=7.5 (Mediatech), 10 mM KCl (Sigma Aldrich), 2 mM MgCl₂ (Sigma Aldrich), and 1 EDTA-free mini protease inhibitor tablet (Pierce) per 10 mL of solution. Dounce homogenization with a tight-fitting pestle or sonication can also be used to disrupt and fractionate the cell membrane. Once the cells have lysed, the cell contents are removed by centrifugation (e.g., centrifuging at 20,000×g). The pelleted material is discarded and the supernatant is centrifuged at 100,000×g and the pellet containing the plasma membrane material is collected. The pelleted membranes are washed once with 10 mM Tris-HCl pH=7.5 and 1 mM EDTA, and then physically serially extruded through a 400 nm, 200 nm, 100 nm and 50 nm polycarbonate membrane. The resulting membrane fragments were then coated onto AAVs by co-extruding the membrane fragments and AAVs were coextruded through a 50 nm polycarbonate membrane.

Determining AAVs are coated with membranes using transmission electron microscopy (TEM). Purified membranes are characterized by using transmission electron microscopy (TEM) by negatively staining with uranyl acetate. Briefly, a drop of membrane solution (at 1 mg/mL) is deposited onto glow-discharged carbon-coated grid; after 5 minutes, the grid is rinsed with 10 drops deionized water; and then 1% uranyl acetate (3 drops) is added to negatively stain membrane particles. The same TEM procedure is then performed with the sample containing AAV and membrane fragments to confirm presence of membrane bound AAVs. Additionally, a control sample of PEG coated AAV can be used. Whereby, the same sizing protocol is used with PEG coated AAVs and AAVs. A Coulter Counter can be further used to measure size of particles.

Fluorescence Quenching Test: The donor cells are linked to a fluorescent probe via a NHS-PEG(2)-maleimide linker. The NHS-PEG(2)-maleimide linker reacts to amine groups of membrane proteins of the donor cells, and also reacts with the probe's thiol group, thereby linking the probe to membrane proteins of the donor cells. The fluorescent probe has the general structure of:

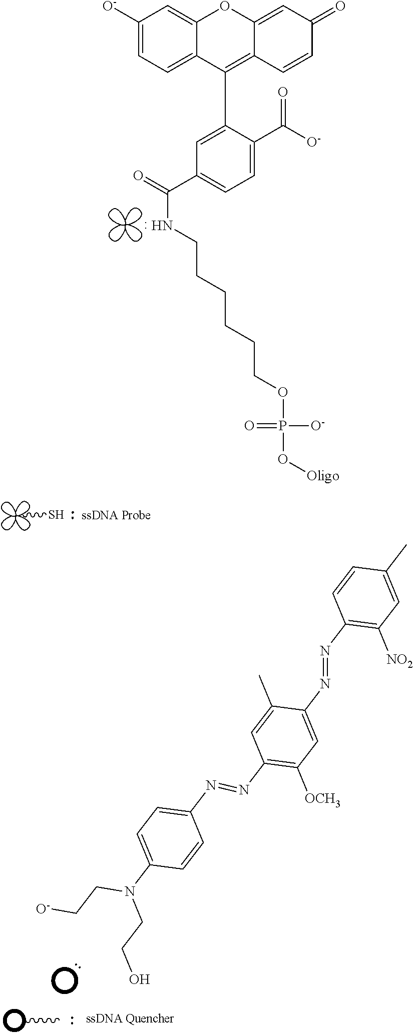

whereby, the 3′ end of the probe has a disulfide attached to the single stranded DNA probe, with the fluorescent dye 6-FAM at the 5′ end. The fluorescent probe is the quenched using a quencher that is attached to a complementary ssDNA sequence.

Determining the protein composition of the membrane: A bicinchoninic acid assay (BCA assay) is first performed in order to get all samples to the same protein concentration of 1 mg/mL in loading buffer (lithium dodecyl sulfate). The samples are heated at 70° C. for 10 mins. 20 uL of samples are loaded into each well of a 4-12% Bis-Tris (buffering agent) 10-well minigel in MOPS running buffer. The gel is then stained with Coomassie blue, and washed by keeping the stained gel in water over night. The washed gel is then imaged to give a general protein profile, and is further compared with a stained gel from the donor cell type. The gel is then transferred to nitrocellulose membrane and western blot is performed for the specific markers using horseradish peroxidase (HRP)-conjugated anti-mouse IgG or anti-rabbit IgG antibodies to Pan-cadherin (membrane marker), Na/K ATPase (membrane pump), CD47 (membrane marker), Histone H3 (nucleus), Cytochrome c oxidase (mitochondria), and GAPDH (cytosol). For controls, antibodies for markers specific to the donor cells are used; and the protein composition to empty membrane vesicles from the donor cells is evaluated.

Elimination half-life of coated AAVs in serum: For membrane-coated AAVs, the stability of the AAVs in serum is evaluated in serum over several hours (AAVs are suspended in 100% FBS and the absorbance is measured with a microplate reader over several hours), and are compared with uncoated AAVs and PEG coated AAVs.

Long term storage of Coated AAVs: The coated AAVs are lyophilized in 5 wt % sucrose, then reconstituted with water. The sizes of the coated AAVs prior to lyophilization and reconstitution are then compared. The coated AAVs can be further compared to uncoated AAVs and PEG coated AAVs.

Integrity of the membrane after cellular uptake: The viral capsid is tagged with red fluorescent dye; the membrane proteins are tagged with green fluorescent dye. Whether the membrane-bound AAV is fusogenic is determined based upon colocalization of the signals (i.e., overlapping fluorescent signals).

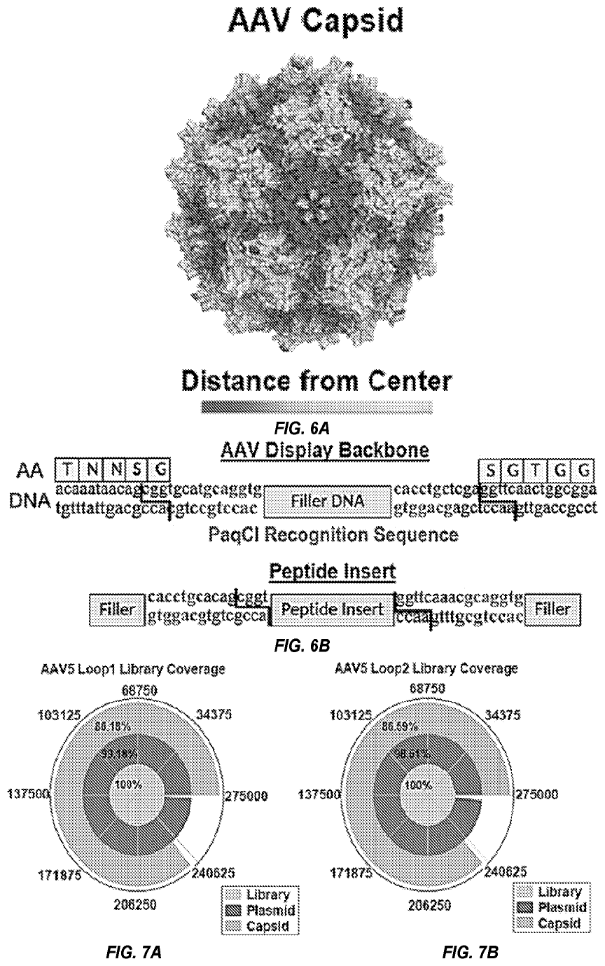

Engineered AAV design: To engineer rationally designed AAV capsids for improved in vivo efficacy and organ specificity, the AAV5 and AAV9 capsids were first modified with peptide insertions at two distinct sites in each serotype. To determine the insertion sites in each of these capsids, amino acid sites at the top of AAV variable regions (e.g., AAV9 VR-VII) were first located within the loop that protrudes from the capsid surface and are important to AAV-cell interactions. For suitable locations for peptide library insertion (see FIG. 6A), positions N443 (AAV5-Loop1) and S576 (AAV5-Loop2) were identified for AAV5, and positions Q456 (AAV9-Loop1) and A587 (AAV9-Loop2) were identified for AAV9. These sites have been linked to the endogenous binding sites of AAV5 to α2-3 N-linked sialic acid and platelet derived growth factor receptor and of AAV9 to galactose and the 37/67 kDa laminin receptor. Accordingly, disruption of these regions via library insertion most likely will inhibit wild-type receptor-mediated cellular interactions. To ensure proper folding and presentation of the peptide library, the peptide was flanked with a 2 amino acid sequence containing a small Glycine-Serine (GS) flexible linker. However, it was further proposed that AAV variants could be engineered with alternative linkers such as longer flexible linkers (e.g., Gly₈) or more rigid linkers (e.g., [EAAAK]₃).

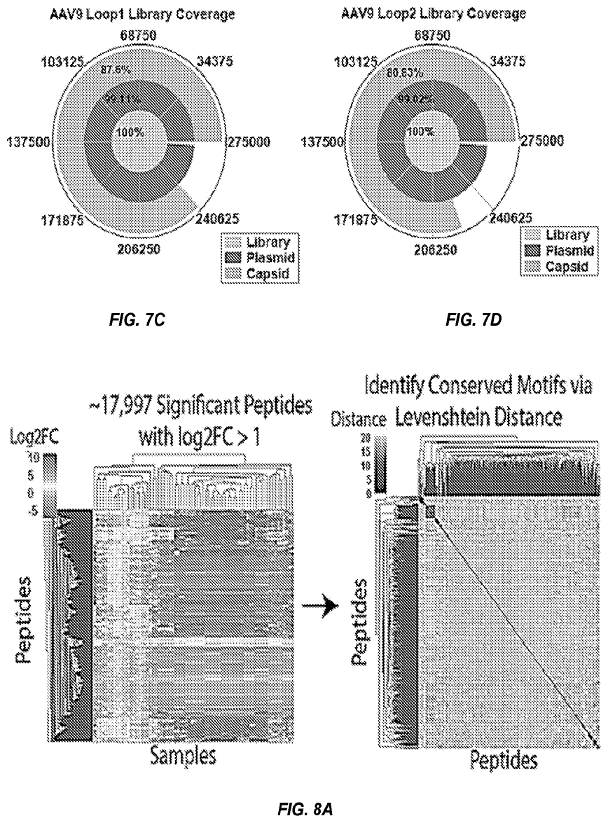

Next. to efficiently clone in the ligand library, a ligation-based approach was utilized with Type IIS restriction enzymes. For this, cloning vectors were created for each of the 4 serotypes in which the target insertion site was modified to contain the flanking GS residues with the PaqCI type IIS recognition sites on either end of an ˜60 base-pair filler DNA sequence. Upon treatment with the PaqCI restriction enzyme, the filler DNA is excised leaving sticky end overhangs which are complementary to the sticky ends generated by PaqCI treatment of the peptide library (see FIG. 6B). These two components can then be ligated together to obtain a plasmid pool which has the peptide library cloned into specific regions of the AAV capsid gene. Through next generation sequencing of this plasmid pool, it was found that greater than 98% of all peptides present in the originally designed library were successfully cloned into each AAV capsid gene using this cloning approach (see FIG. 7A-D).

Furthermore, while the initial screening pool of modified capsids was composed of AAV5 and AAV9 capsids, this approach could be applied to further engineer a broad range of AAV serotypes. This includes, but is not limited to, wild-type AAV serotypes such as AAV1-5 and AAV7-9. as well as the over 100 AAV variants which have been identified in human and non-human primate tissues. Additionally, this approach could be applied to pseudotyped AAV vectors which comprise mixed capsids and genomes from different AAV serotypes, as well as previously engineered AAV variants such as AAV-PHP.eB with improved transduction of neurons and glia in the central nervous system, AAV-PHP.S with improved tropism for neurons within the peripheral nervous system, and AAV-DJ which specializes in highly efficient gene transfer in vivo. Finally, while the initial screen was performed with AAV capsids encapsulating a linear single-stranded DNA genome, this approach could be utilized with self-complementary AAV (scAAV) genomes to enable differing gene expression dynamics in vivo.

Design of displayed peptide library: Each AAV library consisted of 275,298 peptides, derived from 6,465 proteins. These protein sources were mined from a variety of protein families, including all protein ligands cataloged in the Guide to Pharmacology database (Harding et al. 2018), toxins, nuclear localization signals (NLS), viral receptor binding domains, albumin and Fc binding domains, transmembrane domains, histones, granzymes, and predicted cell penetrating motifs (see FIG. 5). In addition to peptides coding for functional biomolecules, 444 control peptides coding for FLAG-tags with premature stop codons were also included. Because the constructs introduce a stop-codon in the AAV capsids, none of them should successfully package or transduce cells in vivo.

Method for identifying infective AAV variants: Utilizing the plasmid pool described above, AAV capsids were generated by transfecting HEK293T cells with the plasmid library pool and an adenoviral helper plasmid at a ratio to prevent capsid cross-packaging. AAVs were purified via iodixanol gradient ultracentrifugation and a subset of the capsids were subjected to next generation sequencing to determine which peptide inserts were successfully packaged. Greater than 80% of the starting peptide pool was identified across all the serotypes (see FIG. 7A-D).

Each AAV capsid pool was then diluted to administer a final viral dose of 0.5E12 viral genomes/mouse for AAV5 libraries, and 1E12 viral genomes/mouse for AAV9) libraries. AAV libraries were delivered via retro-orbital injections to two mice for each AAV serotype library. Following a two-week transduction period in vivo the liver, brain, skeletal muscle, large intestine, spleen, kidney, lungs, heart, and pancreas were collected from each mouse. DNA was isolated from each tissue using TriZol extraction and the region of the AAV capsid containing the peptide insert was selectively PCR-amplified and prepared for next generation sequencing (e.g., see FIG. 5). Once sequenced, the total count of each peptide was quantified across all organs for each serotype and normalized relative to the average read depth of all the samples.

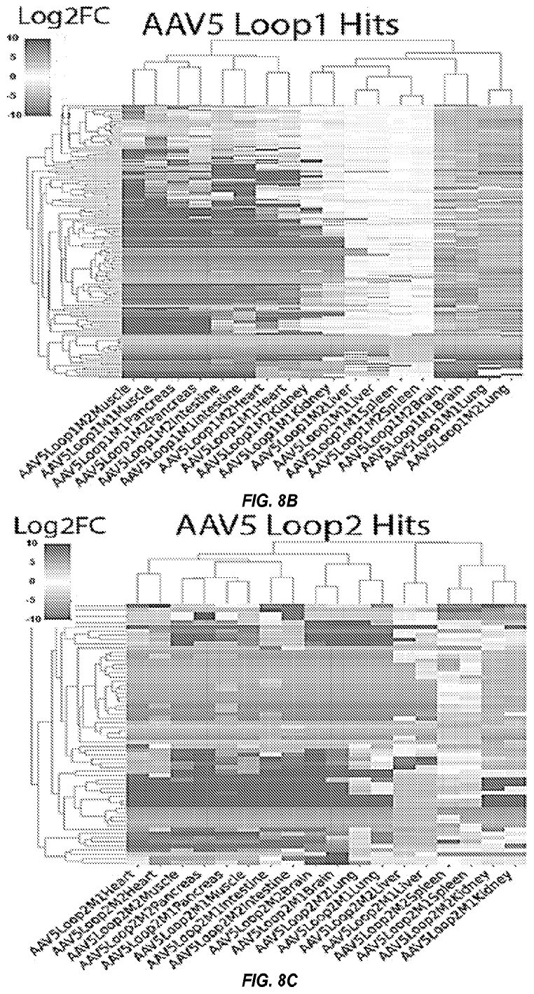

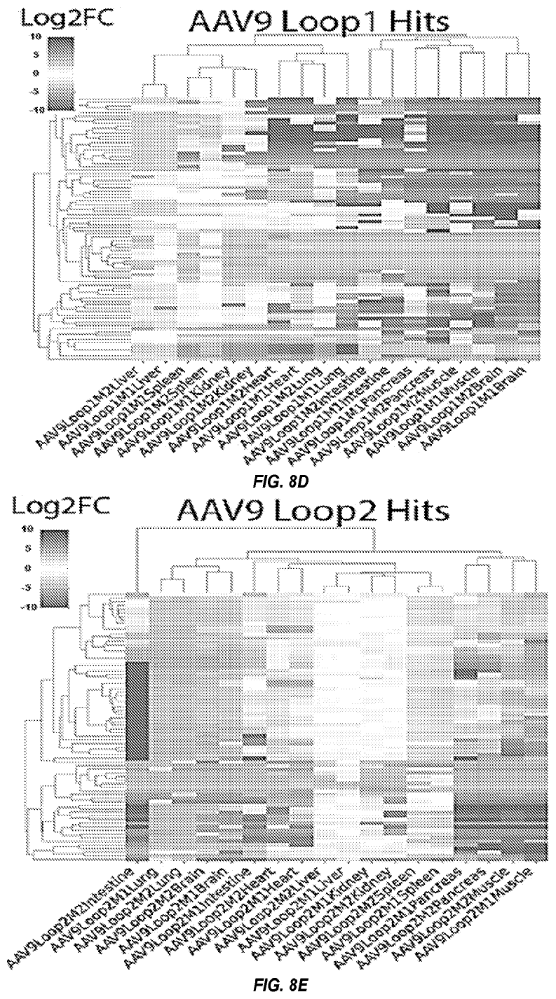

To analyze the top transducing AAV variants in each organ, the log2(fold-change) (log2FC) value was then calculated for each organ and then compared to the count of that AAV variant in the capsid pool along with a significance value calculated using a one-sample t-test (see FIG. 8A). It was found in all organs that over 17,000 AAV variants which have an FDR adjusted p-value<0.05 and a log2FC greater than 1. As expected, many of the significant hits targeted the liver, and surprisingly, several hits appear to transduce all tissues (see FIG. 8A). To identify the absolute top performing AAV variants, a series of filtering steps were devised to stratifs variants. First, the Levenshtein distance was calculated between ‘hit’ peptides to identify motifs that are consistently able to successfully transduce in vivo (see FIG. 8A). To filter the ˜18.000 initial hits, only ‘hit’ peptides were considered where a similar (>50% sequence homology) peptide was detected among the hit pool. The goal of this filtering is to ensure that the ‘hit’ peptide motifs are internally reproducible Second, to ensure that the high increase in counts across organs was due to a strong transduction capability rather than low count values in the capsid pool, out all AAV variants were filtered out with a log2count value less than 3 in the capsid pool. Finally, a log2FC derived Z-score was calculated for each organ sample, to rank how each peptide performs relative to all others in a particular organ. Peptides were only considered with a Z-score>2.5 in at least one organ. The filtering process resulted in a list of 400 top performing displayed-peptides across the four AAV capsids/loops. Log2FC values for these top performing hits are shown in FIG. 8B-E.

A subset of 112 AAV additional variants which appear to be strong transducers across organs were also identified (see FIG. 9A). These additional variants were identified by taking the average transduction level across all organs, and ranking the AAV variants on this metric.

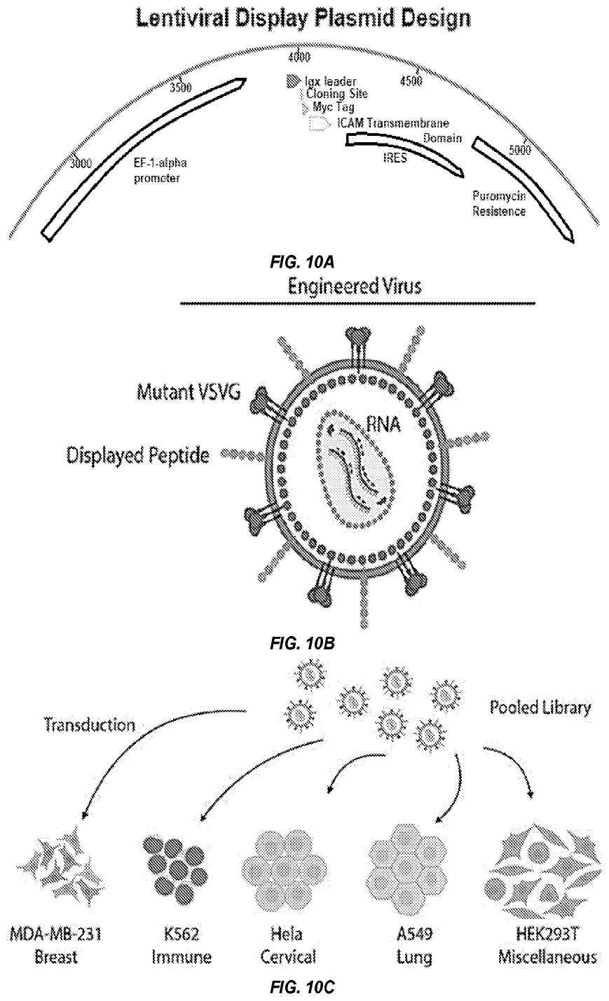

Engineered lentiviral design: In addition to screening engineered AAVs, this methodology was adapted to engineering peptide-decorated lentiviral particles. To display peptides on lentiviral particles, a plasmid was built which contains the expression machinery to constitutively express peptides tethered to an ICAM1 transmembrane domain, which has previously been shown to associate with budding lentiviral particles (see FIG. 10A). An identical library of peptides as in the AAV screens was then cloned into this lentiviral display backbone. The library plasmid also contained a puromycin resistance gene, which could be used to select successfully infected cells via the addition of puromycin to the cell culture media. This plasmid library of peptides was used, along with a double mutant VSVG plasmid (K47Q and R354Q), to produce a library of lentiviral particles in HEK293T cells (see FIG. 10B). The double mutant VSVG retains the ability to promote fusion of the lentiviral particles to the target cells, but is devoid of native receptor binding capabilities. For lentiviral production, cells were transfected with a 1:100 dilution of the transfer vector to prevent cross-packaging of peptide genomes to the incorrect lentiviral particle.

Method for identifying infective lentiviral variants: To identify cell type specific lentiviral variants, five cell lines were subjected to targeted transduction using the library of peptide-displaying lentiviral particles (see FIG. 10C). These cells were chosen to span a variety of tissue types, with the goal of identifying tissue specific lentiviral particles. The cells were transduced overnight in complete DMEM media, and changed to puromycin containing media after 48 h. After 3 days of selection in puromycin containing media, genomic DNA was isolated from the surviving cells via a Qiagen DNeasy genomic DNA isolation kit. Genomic DNA was then used as a template for PCR reactions (5 μg gDNA/100 μl PCR reaction) to amplify the integrated DNA coding for the displayed peptides. A second PCR was then performed to attach indices and prep the samples for sequencing on an Illumina NovaSeq. Deep sequencing was then performed to identify peptide variants which promote infection of each of the above-mentioned cell types.

It will be understood that various modifications may be made without departing from the spirit and scope of this disclosure. Accordingly, other embodiments are within the scope of the following claims.

1. A viral vector having a capsid protein comprising a heterologous targeting peptide in a range of 10-30 amino acids in length.

2-3. (canceled)

4. The viral vector of claim 1, wherein the viral vector is an adeno-associated virus (AAV) or a lentiviral vector.

5. (canceled)

6. The viral vector of claim 1, wherein the capsid protein is a VP1, VP2 or VP3 capsid protein.

7-8. (canceled)

9. The viral vector of claim 4, wherein the heterologous targeting peptide is inserted into an AAV capsid protein at loop 1 and/or loop 2.

10. The viral vector of claim 4, wherein the viral vector is an AAV5 or AAV9.

11. (canceled)

12. The viral vector of claim 10, wherein the heterologous targeting peptide is flanked by a linker peptide at the N-terminal and C-terminal ends of the heterologous targeting peptide.

13. (canceled)

14. The viral vector of claim 1, wherein the heterologous targeting peptide targets the viral vector to a cell selected from the group consisting of hepatocytes, neuronal cells, pancreatic cells, cardiac cells, renal cells, and muscle cells.

15. The viral vector of claim 1, wherein the heterologous targeting peptide targets the viral vector to a tissue selected from the group consisting of brain tissue, liver tissue, pancreas tissue, heart tissue, lung tissue, intestinal tissue, spleen tissue, kidney tissue and muscle tissue.

16-22. (canceled)

23. An adeno-associated virus (AAV) capsid protein comprising a heterologous targeting peptide cloned into loop 1 and/or loop 2 of the capsid protein, wherein the heterologous targeting peptide is about 10-30 amino acids in length.

24. The AAV capsid protein of claim 23, wherein the capsid protein is a VP1, VP2 or VP3 capsid protein.

25-28. (canceled)

29. The AAV capsid protein of claim 23, wherein the heterologous targeting peptide is flanked by a linker peptide at the N-terminal and C-terminal ends of the heterologous targeting peptide.

30. The AAV capsid protein of claim 23, wherein the heterologous targeting peptide targets a cell selected from the group consisting of hepatocytes, neuronal cells, pancreatic cells, cardiac cells, renal cells and muscle cells.

31. The AAV capsid protein of claim 23, wherein the heterologous targeting peptide targets a tissue selected from the group consisting of brain tissue, liver tissue, pancreas tissue, heat tissue, lung tissue, kidney tissue, intestinal tissue, spleen tissue and muscle tissue.

32-38. (canceled)

39. A recombinant AAV (rAAV) comprising a capsid protein of claim 23.

40. A recombinant AAV (rAAV) of claim 39 comprising a capsid protein having a targeting peptide in loop 1 and/or loop 2 wherein the targeting peptide is independently selected from SEQ ID Nos:5865 to 11445.

41. The recombinant AAV of claim 40, wherein the recombinant AAV further comprises a heterologous polynucleotide for gene delivery.

42. The recombinant AAV of claim 41, wherein the heterologous polynucleotide is a therapeutic gene.

43. A composition comprising the rAAV of claim 40.

44. The composition of claim 43 further comprising a pharmaceutically acceptable carrier.

45. A method for delivering a transgene to a subject comprising:
administering a recombinant AAV (rAAV) to a subject, wherein the rAAV comprises:
(i) a capsid protein of claim 23, and
(ii) at least one transgene, and wherein the rAAV infects cells of a target tissue of the subject.

46. The method of claim 45, wherein the at least one transgene encodes a protein an siRNA or an miRNA.

47. The method of claim 46, wherein the protein is an immunoglobulin heavy chain or light chain or fragment thereof.

48-52. (canceled)

53. The method of claim 45, wherein the transgene expresses a transcript that comprises at least one binding site for a miRNA, wherein the miRNA inhibits activity of the transgene, in a tissue other than the target tissue, by hybridizing to the binding site.

54. The method of claim 45, wherein the at least one transgene encodes a gene product that mediates genome editing.

55. The method of claim 45, wherein the transgene comprises a tissue specific promoter or inducible promoter.

56. (canceled)

57. The method of claim 45, wherein the rAAV is administered intravenously, intravascularly, transdermally, intraocularly, intrathecally, orally, intramuscularly, subcutaneously, intranasally, or by inhalation.

58. The method of claim 45, wherein the subject is selected from a mouse, a rat, a rabbit, a dog, a cat, a sheep, a pig, a human and a non-human primate.

59. (canceled)

60. An isolated nucleic acid encoding an AAV capsid protein containing an amino acid sequence selected from the group consisting of SEQ ID No:5865 to 11444 and 11445.

61-62. (canceled)

63. A kit for producing a rAAV, the kit comprising:
a container housing an isolated nucleic acid of claim 60.

64. (canceled)

65. The kit of claim 63, further comprising at least one container housing a recombinant AAV vector, wherein the recombinant AAV vector comprises a transgene.

66. A method for coating a virus or viral particle with membrane fragments comprising:
lysing donor cells in a hypotonic solution, which optionally may be combined with Dounce homogenization or sonication, in order to fractionate the cell membrane;
removing cells and cell debris by one or more rounds of centrifugation, leaving a membrane enriched fraction;
extruding the membrane enriched faction through polycarbonate membrane(s) to generate purified membrane fragments; and
coating virus or viral particles by coextruding the virus or viral particles with the purified membrane fragments through polycarbonate membrane(s).

67. The method of claim 66, wherein the viruses or viral particles are non-enveloped viruses or viral particles.

68. The method of claim 66, wherein the viruses or viral particles are enveloped viruses or viral particles which have had their viral envelope removed.

69. (canceled)

70. The method of claim 66, wherein the viruses or viral particles are adeno-associated viruses (AAV).

71. The method of claim 66, wherein the viruses or viral particles have been modified by directed evolution to have increased neutralizing antibody-evasion properties, as well as enhanced gene delivery, gene targeting, and/or enhanced capacity to infect.

72. The method of claim 66, wherein the viruses or viral particles have been modified by one or more amino acid substitutions in one or more regions of a viral capsid protein so as to reduce the affinity of the viral capsid protein for the major histocompatibility complex.

73. The method of claim 66, wherein the donor cells are mammalian cells.

74. (canceled)

75. The method of claim 73, where the donor cells are human stem cells, human progenitor cells, human primary cells, human somatic cells, human germline cells, or human tumor cells.

76. The method of claim 66, wherein the membranes of the donor cells have been modified to express or present a targeting ligand.

77. The method of claim 76, wherein the targeting ligand is used to improve entry of the coated viruses or viral particles into target cells, inhibit components of the immune response to the coated viruses or viral particles, or to target the coated viruses or viral particles to certain cell types or organs.

78. (canceled)

79. The method of claim 76, wherein the targeting ligand comprises a peptide of any one of SEQ ID Nos:5865 to 11445.

80. Coated viruses or viral particles made by the method of claim 66.

81-86. (canceled)

87. A method of preparing an engineered retroviral particle, the method comprising treating a retroviral particle with a detergent to remove a lipid envelop to obtain naked retroviral particles, isolating the naked retroviral particles and co-extruding a lipid envelop with the naked retroviral particles to obtain an engineered retroviral particle.
